(19) United States
(12) Patent Application Publication
(10) Pub. No.: US 2009/0004658 A1
(43) Pub. Date: Jan. 1, 2009

(54) INTEGRIN ALPHA 7 MUTATIONS IN PROSTATE CANCER, LIVER CANCER, GLIOBLASTOMA MULTIFORME, AND LEIOMYOSARCOMA

(76) Inventor: Jianhua Luo, Wexford, PA (US)

Correspondence Address: JESSE A. HIRSHMAN, ESQ., 1722 MURRAY AVENUE, THIRD FLOOR, PITTSBURGH, PA, 15217, US

(21) Appl. No.: 12/112,657

(22) Filed: Apr. 30, 2008

Related U.S. Application Data
(60) Provisional application No. 60/926,854, filed on Apr. 30, 2007.

(51) Int. Cl. C12Q 1/68; C12Q 1/02

(52) U.S. Cl. 435/6; 435/29

(57) ABSTRACT

Methods are provided for determining the presence of a cancer in a biological sample, such as a tissue biopsy. The methods comprise determining if integrin alpha 7 expression is decreased in the biopsy which is indicative of the presence of a cancer or likelihood of relapse of a cancer. This can be accomplished by determining if levels of integrin alpha 7 mRNA or protein are decreased as compared to a control. This also can be accomplished by determining if a mutation in the integrin alpha 7 gene is present in the biopsy.

### CROSS-REFERENCE TO RELATED APPLICATIONS

This application claims the benefit under 35 U.S.C. § 119(e) to U.S. Provisional Patent Application No. 60/926,854, filed Apr. 30, 2007, which is incorporated herein by reference in its entirety.

### STATEMENT REGARDING FEDERAL FUNDING

The present invention was made with government support under Grant Nos. IU01CA88110-01, R01-GM65188, R01-CA098249 from the National Cancer Institute. The government has certain rights in the invention.

### BACKGROUND

Integrins are the major adhesive molecules in mammalian cells. Each integrin subtype plays a unique role in cell differentiation and embryo development. However, integrin involvement in carcinogenesis has not been well defined.

As a major class of adhesive molecules in mammalian cells, the integrins are involved in many cellular processes, including development, immune responses, leukocyte traffic, and hemostasis (Hynes R O. Integrins: bidirectional, allosteric signaling machines. Cell 2002; 110(6):673-87). Integrin knock-out mice have distinctive developmental defects, including kidney tubule defects, severe skin blistering, chylothorax, and muscular dystrophy (Pulkkinen L, Uitto J. Mutation analysis and molecular genetics of epidermolysis bullosa. Matrix Biol 1999; 18(1):29-42; Fassler R, Meyer M. Consequences of lack of beta 1 integrin gene expression in mice. Genes Dev 1995; 9(15): 1896-908; Georges-Labouesse E, et al. Essential role of alpha 6 integrins in cortical and retinal lamination. Curr Biol 1998; 8(17):983-6; Kreidberg J A, et al. Alpha 3 beta 1 integrin has a crucial role in kidney and lung organogenesis. Development 1996; 122(11):3537-47; McHugh K P, et al. Mice lacking beta3 integrins are osteosclerotic because of dysfunctional osteoclasts. J Clin Invest 2000; 105(4):433-40; Taverna D, et al. Dystrophic muscle in mice chimeric for expression of alpha5 integrin. J Cell Biol 1998; 143(3):849-5).

The integrin superfamily contains 24 members, each of which mediates a unique function in mammals. For example, integrins α3, α6, or α7 combine with a β1 subunit to form receptors for laminin; and the combination of a β1 subunit with α1, α2, α10, or α11 forms a receptor for collagen; heterodimers between β2 and αL, αM, αX, and or αD form leukocyte-specific receptors; and heterodimers between αV and several β subunits form the RGD tripeptide receptor. Regulation of integrin expression is critical for certain aspects of tissue differentiation and regeneration [e.g., keratinocyte differentiation, hair follicle formation, and skeletal muscle development (Brakebusch C, et al. Skin and hair follicle integrity is crucially dependent on beta 1 integrin expression on keratinocytes. Embo J 2000; 19(15):3990-4003; Werner A, et al. Impaired axonal regeneration in alpha7 integrin-deficient mice. J Neurosci 2000; 20(5):1822-30; Mayer U, et al. Absence of integrin alpha 7 causes a novel form of muscular dystrophy. Nat Genet. 1997; 17(3):318-23)], and abnormal integrin expression is associated with several human diseases [e.g., muscular dystrophy, Glanzmann thrombasthenia, and congenital cardiac myopathy (Mayer U, et al. Nat Genet. 1997; 17(3):318-23; Basani R B, et al. A naturally occurring mutation near the amino terminus of alphaIIb defines a new region involved in ligand binding to alphaIIbeta3. Blood 2000; 95(1): 180-8; Hayashi Y K, et al. Mutations in the integrin alpha7 gene cause congenital myopathy. Nat Genet. 1998; 19(1):94-7)]. Integrin α7 is thought to be involved in smooth and skeletal muscle development (Mayer U, et al. Nat Genet. 1997; 17(3):318-23; Flintoff-Dye N L, et al. Role for the alpha7beta1 integrin in vascular development and integrity. Dev Dyn 2005; 234(1):11-21). Very little is known about the role of integrin α7 in other tissues and organs.

Integrin α7 forms a heterodimer with integrin β1 in the plasma membrane and is responsible for communication between extracellular matrix and muscle cells (Echtermeyer F, et al. Specific induction of cell motility on laminin by alpha 7 integrin. J Biol Chem 1996; 271(4):2071-5). There are two distinct isoforms of integrin α7 that are generated by two mutually exclusive alternative splicing (see, e.g., von der Mark, H. et al., Alternative Splice Variants of 71 Integrin Selectively Recognize Different Laminin Isoforms J. Biol. Chem., Vol. 277, Issue 8, 6012-6016, Feb. 22, 2002). Whether integrin α7 has a role in the development of cancer is largely unknown. However, the expression of integrin α7 has been shown to be altered in some malignances [e.g., human leiomyosarcoma and prostate cancer (LaTulippe E, et al. Comprehensive gene expression analysis of prostate cancer reveals distinct transcriptional programs associated with metastatic disease. Cancer Res 2002; 62(15):4499-506; Luo J H, et al. Gene expression analysis of prostate cancers. Mol Carcinog 2002; 33(1):25-35; Yu Y P, et al. Gene expression alterations in prostate cancer predicting tumor aggression and preceding development of malignancy. J Clin Oncol 2004; 22(14):2790-9; Rice J. Mathematical Statistics and Data Analysis: Duxbury Press; 2006)].

### SUMMARY

As described below, various associations between integrin α7 (“integrin alpha 7” or “ITGA7”) in human malignancies have been identified. These associations indicate an increased risk of cancer or an increased risk of cancer relapse. In one embodiment, an increased frequency of mutations in the integrin α7 sequence indicates an increased risk of being diagnosed with cancer. In another embodiment, a lower expression of integrin α7 indicates a higher risk of cancer relapse. In addition, integrin α7 has tumor suppressor activity and inhibits cell motility, where possible targets for this activity are cyclin D kinase inhibitor 3 and GTPase-activating protein.

Methods of diagnosing cancer in human patients are provided. For example and without limitation, these methods can be used to diagnose one or more of prostate cancer, glioblastoma multiforme, leiomyosarcoma, or hepatocellular carcinoma. In one non-limiting embodiment, diagnosing cancer includes one or more of determining the presence of cancerous tumors, the odds ratio and confidence intervals of the diagnosis, the stage of metastasis, the survival estimate, and the likelihood of relapse.

In one non-limiting example, the method comprises determining if human integrin alpha 7 expression is reduced in cells of a biopsy from a patient is decreased, as compared to a normal control, to a level indicative of a cancer, wherein a decrease in human integrin alpha 7 function in the biopsy is indicative of cancer cells in the biopsy. In one example, the decrease in integrin alpha 7 expression in the biopsy indicative of cancer cells in the biopsy is a reduction in integrin alpha 7 mRNA levels to 50% or less of levels present in a normal control. Immunohistochemical methods also can be used to determine relative expression of integrin alpha 7. In certain embodiments, the method further comprises determining if cyclin kinase inhibitor 3 expression is decreased at least 50% in the biopsy as compared to a control and/or determining if rac GTPase-activating protein 1 expression is decreased at least 50% in the biopsy as compared to a control.

In another example, determining if there is a decrease in integrin alpha 7 expression in the biopsy indicative of cancer cells in the biopsy is performed by determining the presence of a mutation in integrin alpha 7 in a nucleic acid sample prepared from the biopsy (expression in this case referring to expression of normal, non-mutated integrin alpha 7). In one embodiment, the mutation is a coding mutation, which results in an altered amino acid sequence of the encoded protein. Coding mutations include, without limitation, truncations, insertions, deletions and substitutions, including substitution of the N-terminal Met, resulting in lack of production of the protein. Non-limiting, illustrative examples of specific mutations associated with a cancer are provided in FIG. 2. In one embodiment, the mutation is one of a stop codon or a frameshift mutation in codons 1-1060 of an alpha integrin 7 open reading frame, examples of which include: W1060stop, W1039Stop, W980Stop, Q921Stop, Q759Stop, Q635Stop, R569Stop, Y526Stop, Q453Stop, E350Stop, W334Stop, and a frameshift mutation in or immediately adjacent to (in one or two codons flanking either side of the listed codon) a codon chosen from one of codons 771, 759, 523 502, 393, 351-353, 286 and 11, such as a deletion of nucleotides ctggact in and adjacent to codon 523 (the codon encoding amino acid 523). Other examples of mutations include missense mutations, point mutations, nonsense mutations, deletions, or insertions in the coding sequence of an integrin alpha 7. In certain non-limiting examples, the mutation is located in one of exons 21 and 11 of human integrin alpha 7. Non-limiting examples of other mutations of relevance in integrin alpha 7 of relevance include MIK, G725R a deletion of V137, and a deletion of 7 nucleotides ctggact amino acids about codon 523 causing a frame shift.

The obtaining a nucleic acid sample from the patient and identifying mutations within the integrin alpha 7 region of the nucleic acid sample as compared to a control. The control can be a sample obtained from one or more patients that have not been diagnosed with cancer or a sample that is considered to be a standardized reference sample. The mutations within the integrin alpha 7 region can contain one or more different types of mutations. For example and without limitation, mutations include a missense mutation, nonsense mutation, deletion mutation, insertion mutation, frameshift mutation, termination mutation, or truncation mutation.

Integrin alpha 7 expression can be determined one or more different assays. For example and without limitation, these assays include an immunohistochemical assay, a nucleic acid amplification assay, PCR, a reverse transcriptase PCR (RT-PCR), an isothermic amplification, a nucleic acid sequence based amplification (NASBA), a 5′ fluorescence nuclease assay, a molecular beacon assay, a microarray assay, and a rolling circle amplification assay.

In another embodiment, a kit is provided, comprising packaging containing a container containing a primer adapted to amplify or sequence a portion of an open reading frame of human integrin alpha 7 containing one or more of codons 1, 11, 137, 286, 334, 350, 352, 393, 453, 502, 523, 526, 569, 635, 759, 771, 921, 980, 1036, and 1060, and at least 5 nucleotides flanking those codons.

### BRIEF DESCRIPTION OF THE DRAWINGS

FIG. 1A is an immunoblot analysis of anti-integrin α7 (anti-ITGA7) serum for specificity against integrin α7 (ITGA7) in PC-3 and 1573 cell protein extracts. Data is shown for preimmune serum (lanes 1 and 2), anti-ITGA7 serum (lanes 3 and 4), anti-ITGA7 monoclonal antibody (lanes 5 and 6), or anti-ITGA7 serum depleted of anti-ITGA7 by incubation with ITGA7 peptides (lanes 7 and 8).

FIG. 1B is a fluorescence photomicrograph of immunohistochemically-stained PITT1 cells induced to express integrin α7 with tetracycline. Stains are shown for nuclei (blue) and ITGA7 expression (red).

FIG. 1C is an immunoblot analysis showing co-immunoprecipitation of integrin β1 (ITGB1) and integrin α7 (ITGA7). Data is shown for an immunoprecipitate (“IP”) of tetracycline-induced PITT cell lysates (lane 1), pre-immune serum (lane 2), and anti-ITGA7 serum (lane 3), where anti-ITGB1 antibodies were used as the immunoblot (indicated as “WB”).

FIG. 2A is a diagram showing integrin α7 mutations in human cancers. The top schematic shows the organization of the integrin α7 exons for mutually exclusive exons (red and black boxes), regular exons (green boxes), and unrelated translation from a frameshift produced by nucleotide insertion (open boxes). Descriptions of each mutation are shown in the table. Abbreviations and symbols include: “Leio”=leiomyosarcoma; NA=not applicable; NK=not known; ND=not determined because of lack of matched normal samples; (s)=separate allele; m=month after primary tumor resection; *=three prostate cancer samples with homozygous mutations and six with heterozygous mutation; ** A93T, D697G, H722Y, L948P(s), and M812V(s).

FIG. 2B are histograms of integrin α7 mutations for representative sequences that have point mutations leading to a stop codon (sense primer, Left), insertion leading to a frameshift (antisense primer, Middle), missense mutation from a case containing only missense mutation (sense primer, Right). Mutation is indicated with an arrow. The histograms of sequences from matched normal samples are shown at the top.

FIG. 3A shows photomicrographs of tissues that are immunohistochemically-stained with integrin α7 peptide antiserum. Photomicrographs are shown for normal prostate tissue, prostate cancer (labeled “PC”), normal smooth muscle of small arteriole and vein (“Smooth muscle”), and soft tissue leiomyosarcoma (“STL”).

FIG. 3B is a graph showing relapse-free survival of patients with prostate cancer. The cutpoint used was an integrin α7 score of 0.5 or less versus more than 0.5. Analysis includes only samples with more than 60 months clinical follow-up. P values were from log-rank tests.

FIG. 3C is a graph showing relapse-free survival of patients with leiomyosarcoma. The cutpoint used was an integrin α7 score of 0.5 or less versus more than 0.5. Analysis includes only samples with more than 60 months clinical follow-up. P values were from log-rank tests.

FIG. 4A are graphs showing colony formation analysis of integrin α7-transfected cells after 10 days. Data is shown for control-transfected PC-3 cells (labeled “P4” and “P5”), integrin α7 expression construct-transfected PC-3 cells (“IT4” and “IT8”), control-transfected Du145 cells (“DP1” and “DP2”), integrin α7 expression construct-transfected Du145 cells (“ITDu3” and “ITDu4”), control-transfected SK-UT-1 cells (“PSK1” and “PSK3”), and integrin α7 expression construct-transfected SK-UT-1 cells (“ISK3” and “ISK7”). Data is also shown for control H1299 and H358 cells, which have normal levels of integrin α7 expression. These cells were transfected with scrambled small interfering RNAs (labeled “scramble siRNA”) or transfected with integrin α7 expression inhibiting small interfering RNAs (labeled “ITGA7 siRNA”). Data are the mean and 95% confidence intervals (CIs).

FIG. 4B are graphs showing soft agar anchorage-independent growth analysis of integrin α7-transfected cells after 22 days. Cells were assayed for their ability to grow in soft agar. Data are the means and 95% CIs.

FIG. 4C are graphs showing wound-healing analysis of integrin α7-transfected cells. Cells were assayed for their ability to recover from similarly sized artificial scratches. Data are the mean percentage of area recovered and their 95% CI.

FIG. 5A is an immunoblot analysis for expression of integrin α7 (“ITGA7”) and 3-actin in PC-3 cells (P4 and P5=vector control, and IT4 and IT8=ITGA7-vector), Du145 cells (DP1 and DP2=vector control, ITDu3 and ITDu4=ITGA7-vector), and SK-UT-1 cells (PSK1 and PSK3=vector control, and ISK3 and ISK7=ITGA7-vector). H1299 and H358 were transfected with vectors expressing either scramble small interfering RNA (“Cont”) or integrin α7 specific small interfering RNA (“ITGA7”).

FIG. 5B are photographs of hematoxylin-stained cells from colony formation assays. Data are shown for representative images of cells from FIG. 6A.

FIG. 5C are photomicrographs of cells from anchorage-independent growth in soft agar soft agar colony formation assays. Data are shown for representative images of cells from FIG. 6A.

FIG. 6A is a graph showing reduction of tumor volume of integrin α7-expressing tumor cells in severe combined immune deficiency mice. Clones of integrin α7-expressing PC-3 and Du145 cells and their corresponding controls were assayed for tumor growth in mice within 6 weeks of tumor cells inoculation. The number of mice in each group and its 95% CI are indicated.

FIG. 6B is a graph showing suppression of metastasis in integrin α7-expressing tumor cells. Incidences of metastases from two clones of each cell lineage were tabulated at the end of 6 weeks or at the time of premature deaths.

FIG. 6C are graphs showing Kaplan-Meier survival analyses of severe combined immune deficiency mice bearing the following xenograft tumors: P4 and P5 (control-transfected PC-3 cells); IT4 and IT8 (integrin α7-transfected PC-3 cells); DP1 and DP2 (control-transfected Du145 cells); and ITDu3 and ITDu4 (integrin α7-transfected Du145 cells). P values were from log-rank tests. All statistical tests were two-sided.

FIG. 7A is an immunoblot analysis of integrin α7 (“ITGA7”), cyclin D kinase inhibitor 3 (“CDKN3”), GTPase-activating protein (“RACGAP1”), and β-actin expression. Data is shown for pcDNA4-ITGA7-transfected PC-3 cells (PITT1 and PITT2 clones) with (labeled “I”) or without (labeled “U”) tetracycline treatment; pCMV-ITGA7-transfected SK-UT-1 cells (“ISK3” and “ISK7”); and vector controls of SK-UT-1 cells (“PSK1” and “PSK3”).

FIG. 7B shows immunoblot analysis, soft agar colony formation analysis (y-axis labeled “Number of Colonies”), and cell migration analysis (y-axis labeled “% Area Recovered”) for PITT1 and PITT2 clones. Cells were treated with (+) or without (−) tetracycline (to induce integrin α7) and/or transfected RACGAP1 small interfering RNA (siRNA), CDKN3 siRNA, and/or scrambled siRNA (control), as shown in the bottom of panel. Data are the mean of triplicates; error bars are 95% CIs. For the soft agar colony formation assay, data are the mean of number of colonies formed after 22 days. For the cell migration assay, data are the mean of the percentage of the area recovered after migration for 24 hours (n=5 areas).

FIG. 7C shows immunoblot analysis, soft agar colony formation analysis (y-axis labeled “Number of Colonies”), and cell migration analysis (y-axis labeled “% Area Recovered”) for PSK1, PDSK3, ISK1, and ISK3 cells. Cells were treated with (+) or without (−) tetracycline (to induce integrin α7) and/or transfected RACGAP1 small interfering RNA (siRNA), CDKN3 siRNA, and/or scrambled siRNA (control), as shown in the bottom of panel. Data are the mean of triplicates; error bars are 95% CIs. For the soft agar colony formation assay, data are the mean of number of colonies formed after 22 days. For the cell migration assay, data are the mean of the percentage of the area recovered after migration for 24 hours (n=5 areas).

FIG. 8A provides a genomic sequence for human integrin alpha 7 (exons are labeled and highlighted in gray) (SEQ ID NO: 85), 8B, provides a cDNA sequence of a first splice variant of inhuman integrin alpha 7 (SEQ ID NO: 86), and 8C (SEQ ID NOS: 86 (nucleotide) and 87 (protein)) provides the open reading frame with amino acid sequence for the splice variant of FIG. 8B. FIG. 8D (SEQ ID NO: 88), provides a cDNA sequence of a second splice variant of inhuman integrin alpha 7, and 8E (SEQ ID NOS: 89 (nucleotide) and 90 (protein)) provides the open reading frame with amino acid sequence for the splice variant of FIG. 8D

### DETAILED DESCRIPTION

Disclosed herein are associations between loss of function of the gene integrin alpha 7 (ITGA7) with various human malignancies. Loss of function of integrin alpha 7 includes low expression of integrin alpha 7 or mutations in the primary amino acid sequence of integrin alpha 7 are associated with increased risk of various cancers and increased risk of cancer relapse in human patients. Lowered function of integrin alpha 7 was found to be associated with more advanced stage of metastasis and with increased risk of cancer relapse in human patients. Integrin alpha 7 was found to have tumor suppressing activity in xenograft tumors within an in vivo mouse model. In addition, the targets of integrin alpha 7 were identified, where these targets mediate cell growth and migration inhibition.

We report that mutations in the integrin α7 gene appear to be wide-spread and frequent in human malignancies. We also found by use of RT-PCR that integrin α7 mRNA was readily detected in tissues of 20 normal organs. Moreover, we detected integrin α7 mRNA in all 16 cell lines examined that were derived from tumors of prostate gland, lung, brain, smooth muscle, liver, and kidney. The presence of mutations in cDNA and genomic DNA from tumor samples and the absence of similar mutations in the matched normal samples largely eliminated the possibility of pseudogene mutations because pseudogene should be present in both normal and tumor samples. Thus, the ubiquitous expression of integrin α7 in human organs and widespread mutations of this gene in human cancers raise the possibility that integrin α7 may have a role in the development of many human malignancies.

Methods are therefore provided for diagnosing human patients with an increased risk of cancer or an increased risk of cancer relapse. In one embodiment, a method of determining the presence of cancer cells in a biopsy obtained from a patient (any biological sample comprising cells obtained from a patient), comprising determining if integrin alpha 7 expression in cells of the biopsy is decreased, as compared to a normal control, wherein a decrease in integrin alpha 7 expression in the biopsy is indicative of cancer cells in the biopsy. Cancer is a group of diseases characterized by uncontrolled growth and spread of abnormal cells. “Expression” of integrin alpha 7, refers to the process by which integrin alpha 7 protein is produced in a cell, including the processes of transcription and translation. In one sense, decreased expression refers to lower levels of mRNA transcripts of integrin alpha 7 or other proteins, where applicable (and thus lowered levels of the protein). In another sense, lower levels of integrin alpha 7 or other proteins can be determined by immunohistochemical methods, such as by in situ visualization in microscope slides, or by determining levels in a gel, by for example, Western blots of 1D or 2D gels. Gels and in situ slides can be scanned and transcript or protein levels can be quantified either visually or using suitable slide or gel scanning methods and devices. In yet another sense, coding mutations in the expressed mRNA that result in changes in the primary amino acid sequence of the translated integrin alpha 7 protein, many of which result in production of an integrin alpha 7 protein that is deficient in its function, contributes to lowered expression of normal, non-mutated integrin alpha 7. The normal activity/function of integrin alpha 7 in cells is the ability of integrin alpha 7 to perform in its normal manner in the cells with respect to cell adhesion and/or signaling. Mutations in the open reading frame (ORF, a portion of a genome which contains a sequence of bases that could potentially encode a protein) of the integrin alpha 7 gene that can contribute to loss of expression of integrin alpha 7 in many instances include, without limitation, truncation, such as by a mutation causing a premature stop codon within the open reading frame of integrin alpha 7, resulting in a truncation of the protein as compared to normal or “wild-type” integrin alpha 7, deletion, insertion, substitutions, frameshift and missense mutations.

This increased risk is associated with one of more mutations within the integrin alpha 7 gene. These mutations lead to structural alteration of the integrin alpha 7 protein, such as through: a premature stop codon; a frameshift mutation; insertions, deletions and substitutions of one or more amino acids; or a missense mutation. Various mutations in the integrin alpha 7 coding region have been identified that are associated with increased risk of cancer or relapse. The methods may further comprise determining the expression levels of one or more targets of integrin alpha 7 within the sample. Possible targets for the integrin alpha 7-tumor suppressor activity include cyclin D kinase inhibitor 3 and GTPase-activating protein.

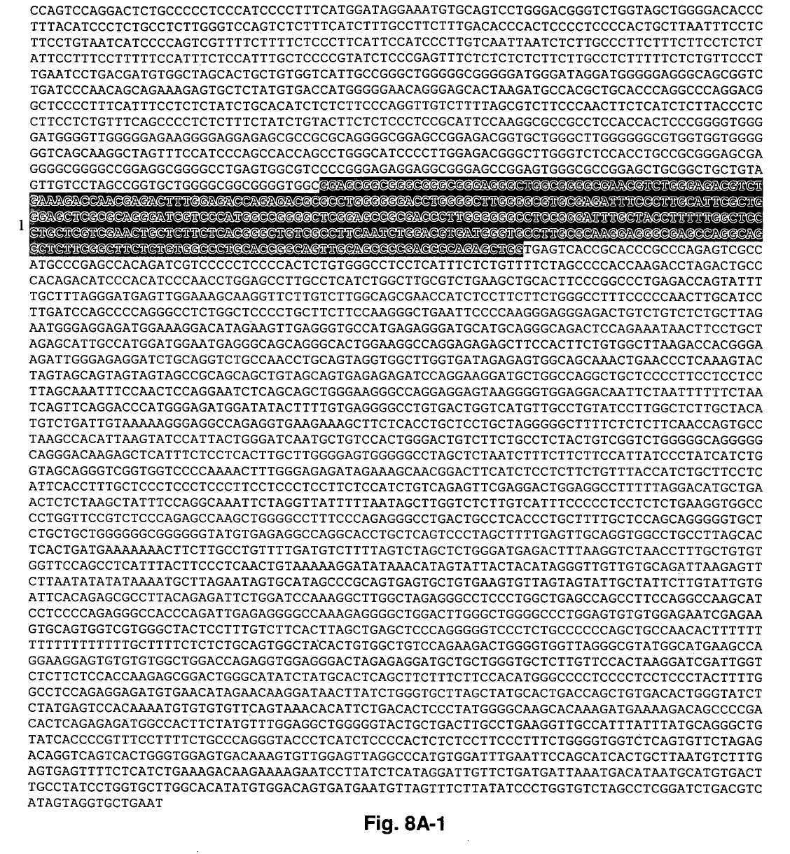

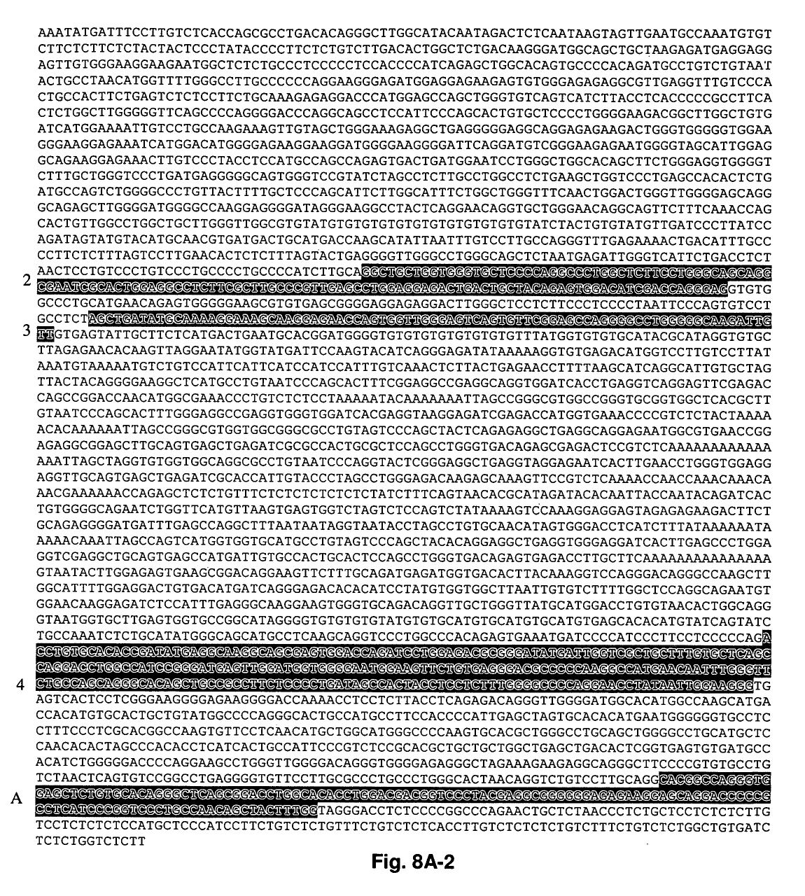

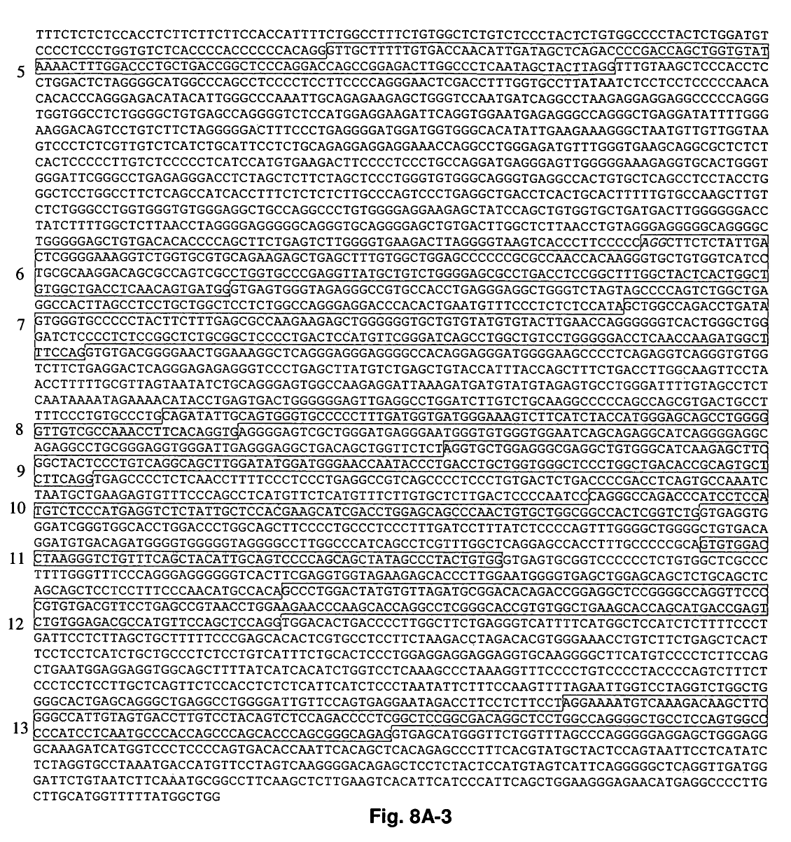

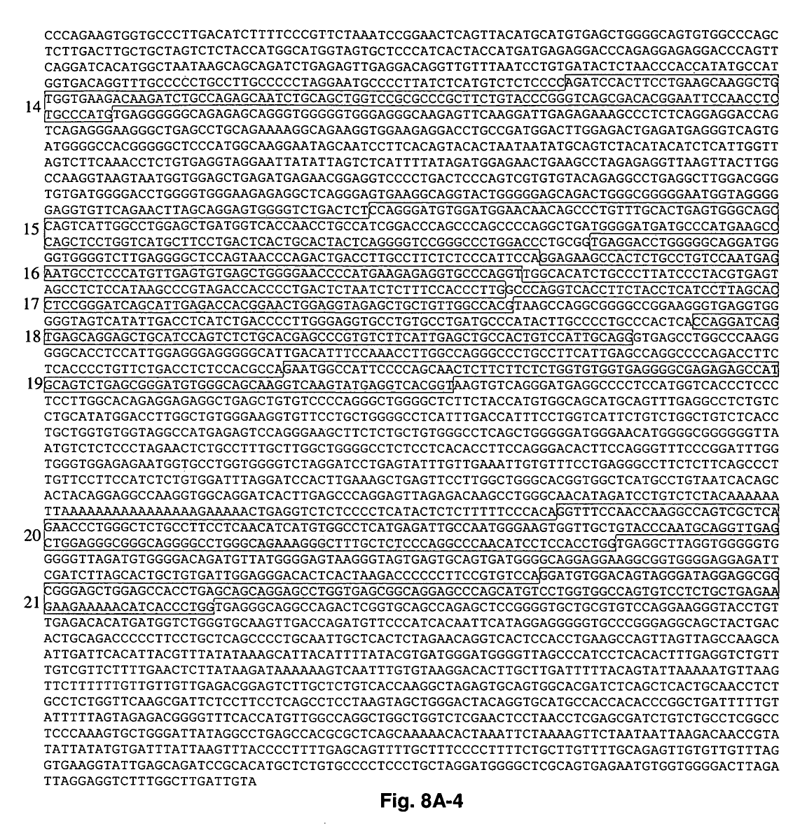

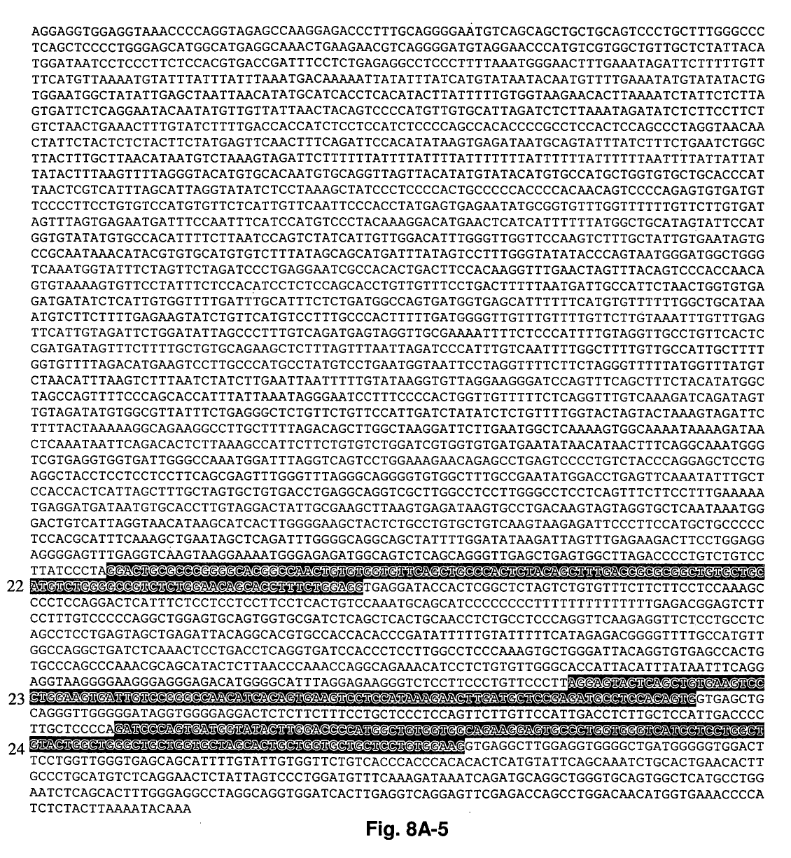

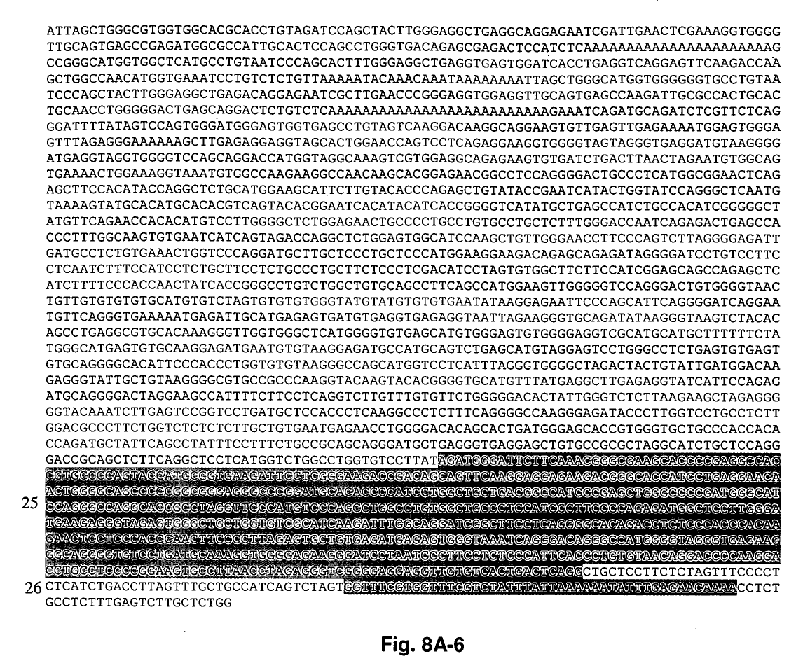

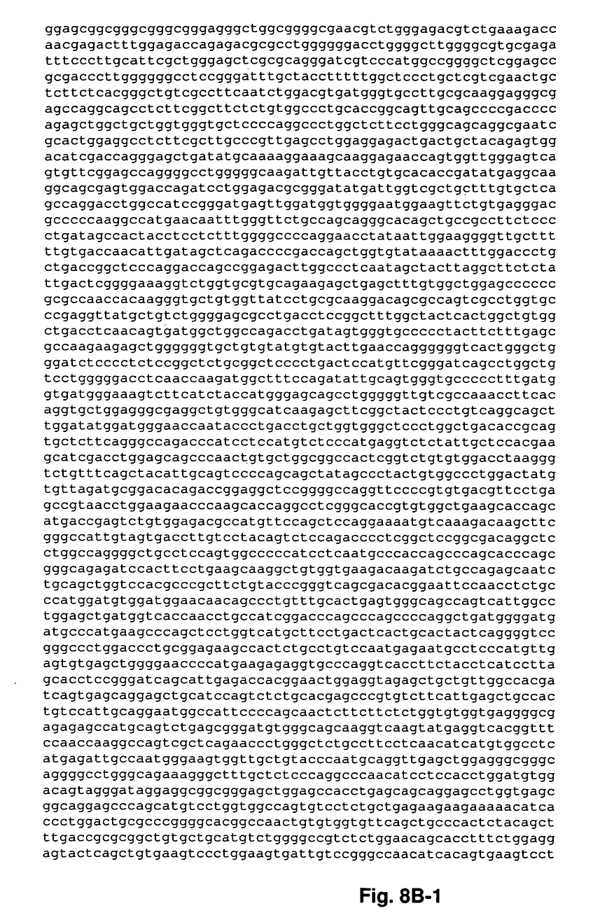

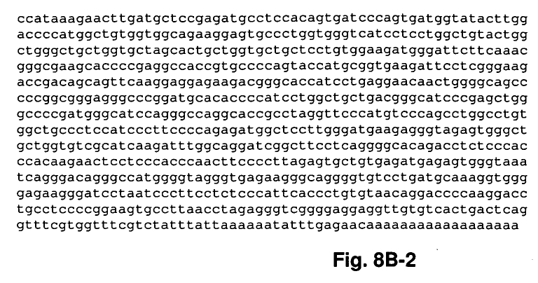

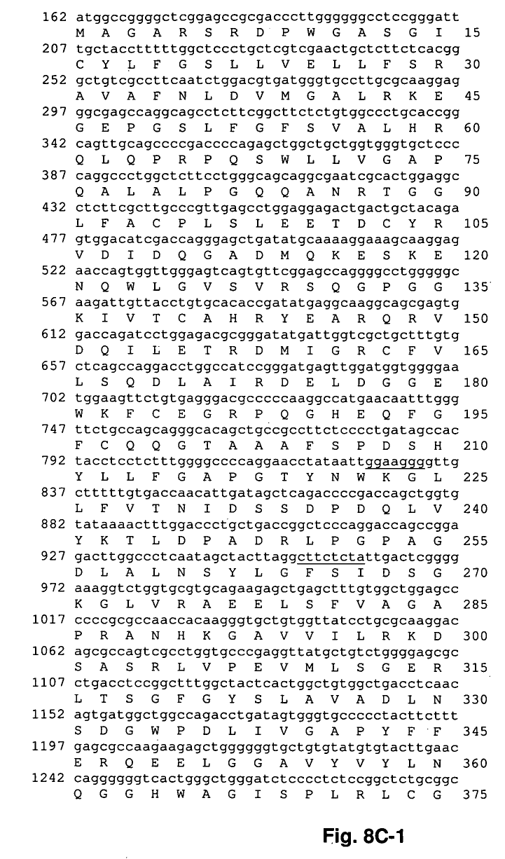

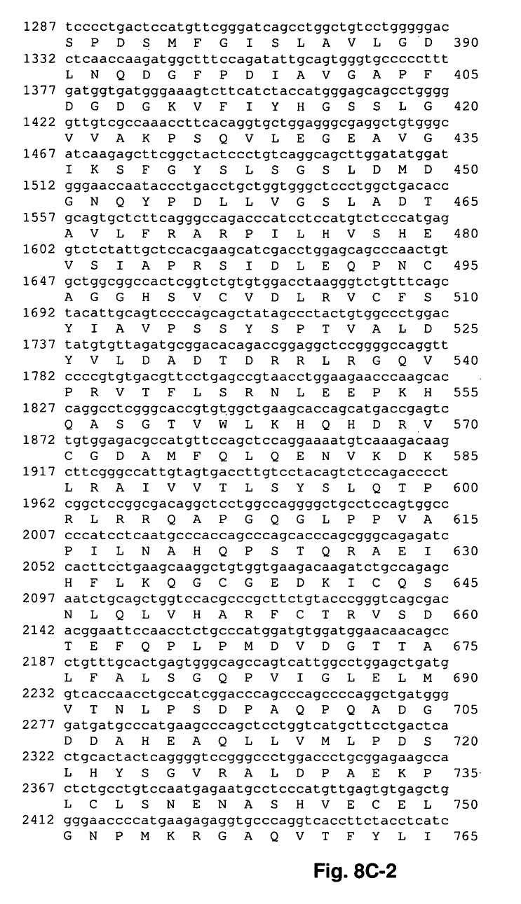

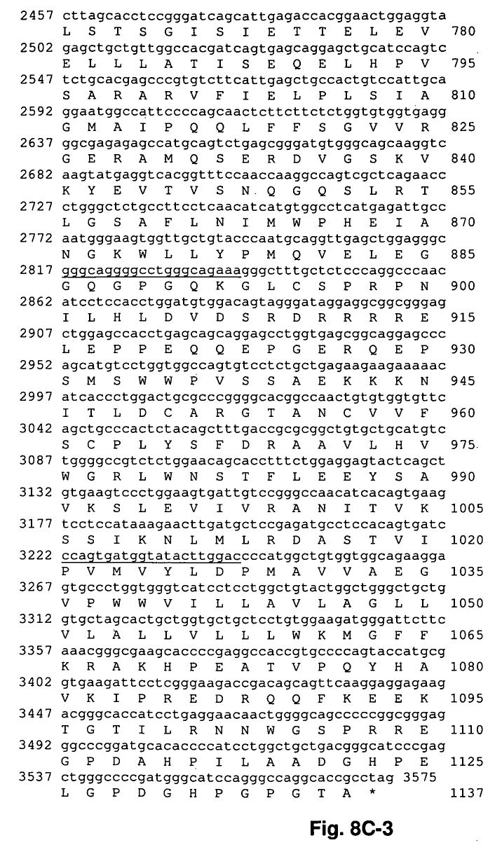

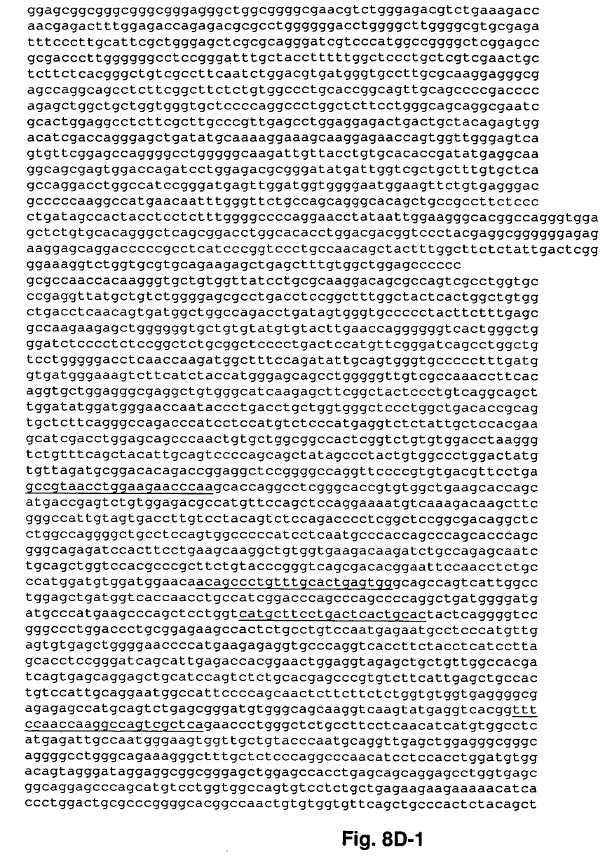

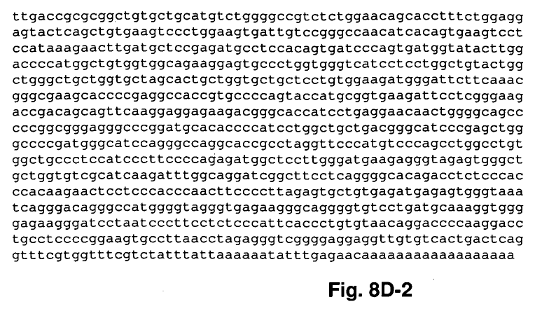

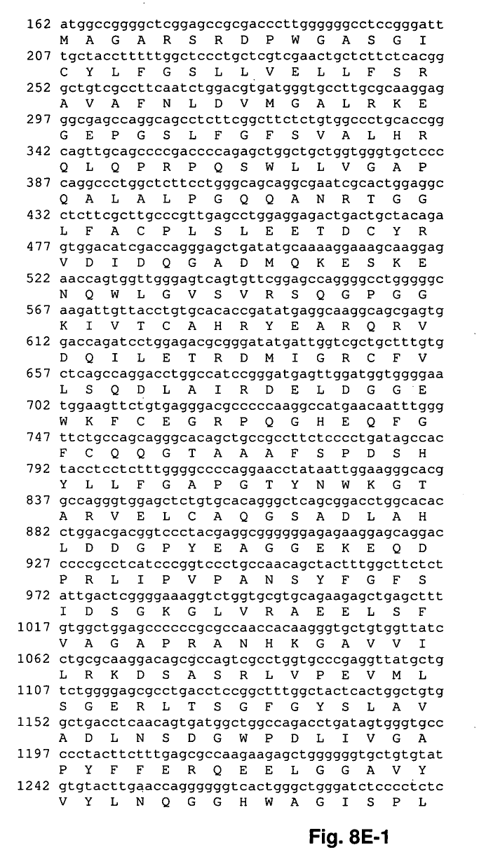

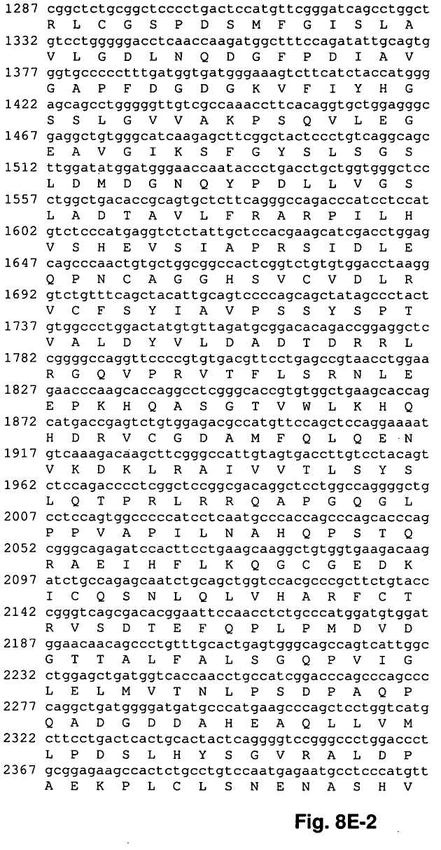

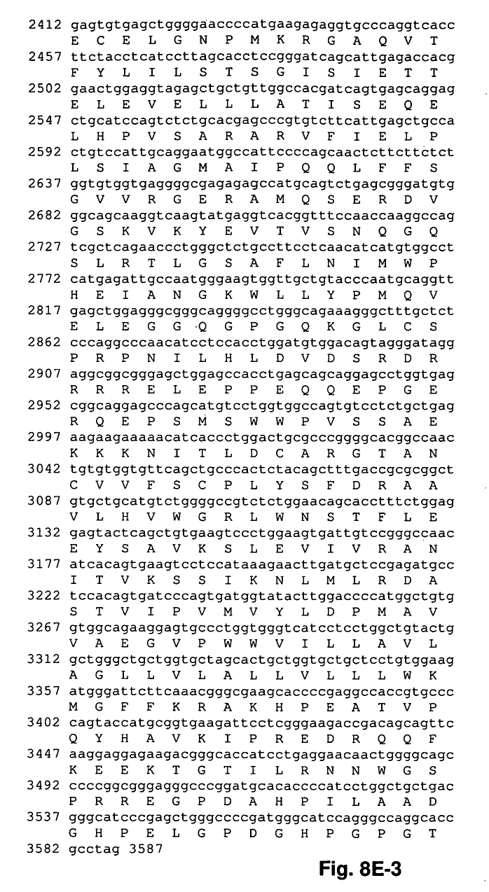

As used herein, a “mutation” refers to a change in the nucleic acid sequence in a subject, such as a human subject. A “coding mutation” refers to a mutation that alters the primary amino acid sequence of a protein. Mutations are determined in relationship to a sequence that is considered “wild-type”, referring to an amino acid or protein sequence or sequences common to many individuals or subjects. Mutations can be identified by comparison to a normal or wild-type sequence, such as, without limitation, those of FIGS. 8A-E, or other sequences that are known or may be found, that exhibit normal integrin alpha 7 function. Coding mutations include single or multiple nucleotide and amino acid substitutions, additions, deletions, including without limitation: point mutations, insertion mutations, deletion mutations, missense mutations, nonsense mutations, frameshift mutations, and truncation mutations.

Unless indicated otherwise, references to specific amino acids, codons or nucleotides are made in reference to the exemplary sequences shown in FIGS. 8B and 8C. Standard nomenclature is used, for example, “MIK” refers to mutation at codon 1 in the Open reading frame (ORF) of integrin alpha 7 which results in Lys being substituted for Methionine (thus resulting in no protein produced). Likewise Q921Stop refers to a mutation at codon 921 which results in termination of the protein instead of insertion of a Gln residue. Although the mutations are indicated in reference to the sequences depicted in FIGS. 8B and 8C, a person of ordinary skill in the art would recognize that these include equivalent mutations in splice variants and normal variations in integrin alpha 7 sequences within the human population as are known or may be recognized in the future.

Mutations other than coding mutations may have any one of a number of effects on protein expression, including without limitation: promoter activity that regulates transcription, which can have the effect of lowering mRNA levels of integrin alpha 7 or which produces altered protein sequences in the final protein product, including frameshift, truncation, protein mis-folding, altered protein processing, destruction (or enhancement) of active sites or binding sites of a protein, mis-splicing of an mRNA or any other property of a nucleic acid sequence affects the expression the final gene products. The integrin alpha 7 gene and transcripts thereof are described, for example and without limitation, in materials associated with the following identification numbers, which are publicly available on-line (see, e.g., GeneID 3679; GenBank Accession Nos. NM_{—}002206.1, NP_{—}002197.1, NC_{—}000012.10, and NT_{—}029419; UniProt Q13683, Q86W93, AND Q4LE35; and MIM (Mendelian Inheritance in Man) 600536). Unless indicated otherwise, in the context of the disclosure herein, integrin alpha 7 is intended to embrace all isoforms thereof, which function in cell adhesion, the lack of which is seen to result in increased cell migration activity, as shown herein.

A normal control for determining levels of integrin alpha 7 mRNA may be an RNA sample prepared from normal tissue obtained from the patient, or other patients, such as a statistically significant pool of RNA samples obtained from multiple normal individuals. A control may be an RNA sample prepared from the same tissue/organ as the biopsy, such as a lymph node, prostate, muscle, etc. Comparison of mRNA levels in the patient's biopsy as compared to a normal control is typically normalized to total RNA quantity and/or to mRNA levels of a reference gene, such as a housekeeping gene, for example 18S rRNA. This may be accomplished by multiplexed RT-PCR or other assays that permit quantification of multiple mRNAs in an RNA sample.

Many statistical analyses were performed to prove that abnormalities of integrin alpha 7 are involved in the progression of human malignancies. Over 700 prostate and 100 leiomyosarcoma samples were tested. Mutations in integrin alpha 7 (or “integrin α7”) were identified by sequencing genomic DNAs and cDNAs from 122 specimens, including 62 primary human tumor samples, four cell lines, and 56 matched normal tissues. A meta-analysis of integrin alpha7 mRNA microarray data from four studies was performed. Kaplan-Meier analyses were used to assess survival. All statistical tests were two-sided.

Integrin alpha7 mutations that generate truncations were found in specimens of 16 of 28 prostate cancers (57%, 95% confidence interval [CI]=37% to 76%), five of 24 hepatocellular carcinomas (21%, 95% CI=7% to 42%), five of six glioblastomas multiforme (83%, 95% CI=36% to 99%), and one of four leiomyosarcomas (25%, 95% CI=0.6% to 81%). Integrin α7 mutations were associated with increased recurrence of human prostate cancer (nine recurrences among 13 patients with integrin α7 mutations vs. one among eight without such mutations; odd ratio [OR]=14, 95% CI=1.15 to 782, P=0.024) and hepatocellular carcinoma (five recurrences among eight patients with integrin alpha7 mutations vs. one among 16 without such mutations, OR=21, 95% CI=1.6 to 1245; P=0.007).

In addition, methods are therefore provided for diagnosing human patients with an increased risk of cancer or an increased risk of cancer relapse, where this increased risk is associated with low expression of integrin alpha 7.

As used herein, the terms “expression” and “expressed” mean production of a gene-specific mRNA by a cell or the production of a protein by a cell. The term “low expression” or “decreased amount of expression” refers to an amount of expression in a sample from a subject that is less than the amount of expression in a control. The control can be a sample obtained from one or more patients that have not been diagnosed with cancer, such as a statistically-relevant population. The control also can be a sample that is considered to be a standardized reference sample, such as a “normal” tissue sample.

Expression of protein can be detected by histological techniques, including immunohistochemical, immunoblotting, and immunofluorescence techniques. Trained histologists can systematically assess the relative difference in expression between a sample and a control. Immunostaining was used to localize and to measure the level of integrin alpha7 in 701 and 141 specimens of prostate and smooth muscle, respectively. Prostate cancer and soft tissue leiomyosarcoma with focal or no integrin α7 expression were associated with reduction of metastasis free-survival.

A large number of methods, including high-throughput methods, are available for detection of mutations and for measurement of expression. In one embodiment, DNA from a sample is sequenced (resequenced) by any method to identify a mutation. A large variety of resequencing methods are known in the art, including high-throughput methods. Amplification-based methods also are available to identify mutations, including, without limitation: PCR, reverse transcriptase PCR (RT-PCR), isothermic amplification, nucleic acid sequence based amplification (NASBA), 5′ fluorescence nuclease assay (for example, TAQMAN assay), molecular beacon assay, FRET-based (fluorescence resonance energy transfer-based) assay and rolling circle amplification. Assays may be multiplexed, meaning two or more reactions are carried out simultaneously in the same physical location, such as in the same tube or position on an array—so long as the reaction products of the multiplexed reactions can be distinguished. As a non-limiting example, TAQMAN or molecular beacon assays can be multiplexed by use of and by monitoring of accumulation or depletion of two different fluorophores corresponding to two different sequence-specific probes. In most cases, the appropriate method is dictated by personal choice and experience, equipment and reagents on hand, the need for high throughput and/or multiplexed methods, cost, accuracy of the method, and the skill level of technicians running the assay. Design and implementation of those techniques are broadly-known and are well within the abilities of those of average skill in the art.

Also provided are kits for performing the above-described assays. In one embodiment, a kit is provided comprising packaging containing a container containing a primer adapted to amplify or sequence a portion of an open reading frame (ORF) of human integrin alpha 7 containing one or more of codons 1, 11, 137, 286, 334, 350, 352, 393, 453, 502, 523, 526, 569, 635, 759, 771, 921, 980, 1036, and 1060, and at least 5 nucleotides flanking those codons. Packaging can be any commercially acceptable packaging, including paper, plastic, foil, glass, etc. A primer adapted to amplify or sequence a specific codon is one or more nucleic acids able to prime an amplification reaction, such as PCR or a sequencing reaction. A portion of the integrin alpha 7 refers to anything other than the entire human alpha integrin sequence. Thus, this specifically excludes random primers or primers that hybridize to nucleic acids other than those of human integrin alpha 7. The portion that is sequenced or amplified contains the indicated codon and surrounding (flanking) bases. The “container” can be any useful device, and includes arrays as are known in the art, such as gene sequencing chips where the primer is attached to a surface of an array. In cases where the array or chip does not contain the primer, the primer may be packaged in a separate container for use in the particular assay for which the array is designed. A large number of arrays, chips, and other high-throughput systems are known in the relevant art, and it is well within the abilities of a person of ordinary skill in the relevant arts to design and configure kits, arrays, primers, primer pairs, probes, etc. that can be employed to sequence or otherwise identify specific polymorphisms in a DNA or cDNA sequence of a gene, such as human integrin alpha 7.

The tumor suppressor activity of integrin alpha 7 was evaluated with various assays, including colony formation, soft agar colony growth, and cell migration assays. Forced expression of normal integrin α7 in prostate cancer and leiomyosarcoma cell lines suppressed tumor growth and metastasis both in vitro and in vivo. Xenograft tumors with increased level of integrin α7 in SCID mice resulted in decreased tumor growth and metastasis. Microarray analysis indicated that cyclin D kinase inhibitor 3 and GTPase-activating protein may be possible targets for integrin alpha7-mediated tumor suppressor activity and inhibition of cell motility. Integrin alpha7 appears to be a tumor suppressor that operates by suppressing tumor growth and retarding migration. Based on this disclosure, integrin alpha 7 may be used as a pharmaceutical target to treat human malignancies or a diagnostic target to guide to manage cancer patients.

### EXAMPLES

The examples show an association between integrin α7 with various cancers. These examples also show the association between integrin α7 and tumorigenesis or metastasis using cell-based assays. Finally, the examples show the tumor suppressing activity of integrin α7 and the targets of integrin α7 that may promote this tumor suppressing activity.

Throughout the examples, the following various statistical methods are used. Confidence intervals for individual proportions were calculated by use of the exact binomial test (function “binom.test” in R package of statistical computer programs), and those for a numerical distribution were calculated with conventional independent and normal assumption [i.e., mean±(1.96×SD/n^{1/2}), where SD=standard deviation and n=sample size] (Rice J. Mathematical Statistics and Data Analysis: Duxbury Press; 2006). Comparison of two proportions was inferred by Fisher's exact test because of relatively small sample size (function “fisher.test” in R package) (Rice J. Mathematical Statistics and Data Analysis: Duxbury Press; 2006). The odds ratio (OR) estimates and the confidence intervals were inferred by conditional maximum likelihood estimate rather than conventional sample odds ratio. Survival was analyzed by the Kaplan-Meier method, and survival curves were compared by use of the log-rank test (Hosmer D W, Lemeshow S. Applied Survival Analysis: Wiley; 2003). All statistical tests were two-sided.

Throughout the examples, various cell lines were used. All cell lines, including PC-3 (prostate cancer), Du145 (prostate cancer), LNCaP (prostate cancer), SK-UT-1 (leiomyosarcoma), H1299 (lung cancer), and H358 (lung cancer), were purchased from American Type Cell Culture (Manassas, Va.). PC-3 cells were cultured with F12K medium supplemented with 10% fetal bovine serum (InVitrogen, Carlsbad, Calif.). Du145 and SK-UT-1 cells were cultured with modified Eagle medium supplemented with 10% fetal bovine serum (InVitrogen). LNCaP, H358, and H1299 cells were cultured with RPMI 1640 medium supplemented with 10% fetal bovine serum (InVitrogen). The 1573 cells, a renal cell carcinoma cell line (ATCC CRL-1573, also known as 293 cells), were cultured with modified Eagle medium supplemented with 10% fetal bovine serum (InVitrogen). SW-33, SW39, SW40, SW61, SW94, and SW95 (glioblastoma multiformes) were obtained from University of Pittsburgh Hillman Cancer Center, and cultured in modified Eagle medium supplemented with 10% fetal bovine serum (InVitrogen).

Throughout the examples, various immunochemical processes were performed. Immunohistochemistry was performed as described previously (Jing L, et al. Expression of myopodin induces suppression of tumor growth and metastasis. Am J Pathol 2004; 164(5): 1799-806) with purified integrin α7 peptide antiserum (1:1000 dilution). Rabbit anti-integrin α7 serum (polyclonal) was raised through immunization of a rabbit with the synthetic peptide GTILRNNWGSPRREGPDAH (SEQ ID NO: 1), which corresponds to amino acids 1097-1115 of human integrin α7. This synthetic peptide was chemically synthesized and purified by high-pressure liquid chromatography at the University of Pittsburgh biotechnology support center. Rabbit antiserum against this peptide was raised by Cocalico Biologicals, Inc. (Reamstown, Pa.). Antibodies against integrin α7 were purified by use of the synthetic peptide and a Carboxylink kit from Pierce (Rockford, Ill.).

Mouse anti-integrin α7 antibody was purchased from Novus Biologicals Inc. (Littleton, Colo.). Mouse anti-cyclin D kinase inhibitor 3 (CDKN3) monoclonal antibody was purchased from Abnova Inc. (Taipei, Taiwan). Goat anti-GTPase activating protein (RACGAP1) antibody (polyclonal) was purchased from Abcam Inc. (Cambridge, Mass.). Goat anti-integrin β1 (polyclonal) and mouse anti-β-actin monoclonal antibodies were purchased from Santa Cruz Biotechnology, Inc. (Santa Cruz, Calif.).

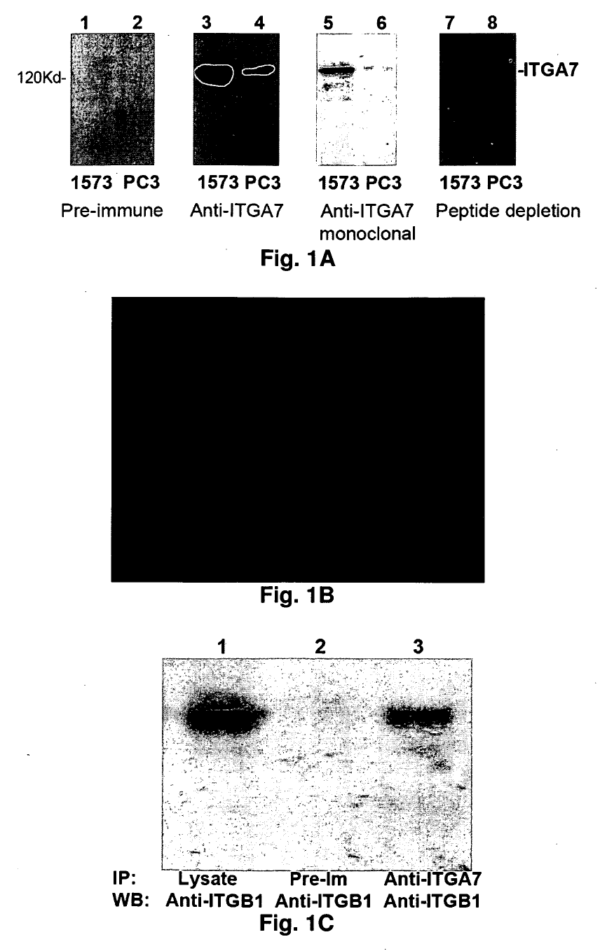

FIG. 1A shows the specificity of rabbit preimmune serum and anti-integrin α7 antiserum on immunoblots of PC-3 and 1573 cell protein extracts. Proteins in lysates of 1573 and PC-3 cells were separated by 8% sodium dodecyl sulfate-polyacrylamide gel electrophoresis (SDS-PAGE) and immunoblotted with preimmune serum (lanes 1 and 2), anti-ITGA7 serum (lanes 3 and 4), anti-ITGA7 monoclonal antibody (lanes 5 and 6), or anti-ITGA7 serum depleted of anti-ITGA7 by incubation with ITGA7 peptides (lanes 7 and 8). Integrin α7 bands were specifically detected in extracts from both 1573 and PC-3 cells with anti-integrin α7 antiserum (as shown in lanes 3 and 4 in FIG. 1A) and with a monoclonal antibody against integrin α7 (a positive control, as shown in lanes 5 and 6 in FIG. 1A). No visible integrin α7 band was detected with either preimmune serum (lanes 1 and 2 in FIG. 1A) or antiserum depleted of integrin α7 peptide antibodies (lanes 7 and 8 in FIG. 1A).

FIG. 1B shows the expression and localization of integrin α7 by immunofluorescence analysis. PITT1 cells, in which integrin α7 expression can be induced by treatment with tetracycline at 1 μg/mL, were used for these experiments. Further information about PITT1 cells are described in Example 3. Cells were grown on covered slides in the presence of tetracycline, fixed with 3% paraformaldehyde, and then blocked with normal donkey serum for 30 minutes at 4° C. Anti-ITGA7 serum or preimmune rabbit serum (as the control) was added, and slides were incubated for 1 hour at 4° C. After three washes with phosphate-buffered saline (PBS), rhodamine-conjugated donkey anti-rabbit secondary antibodies were added and incubated for 1 hour at 4° C. After three washes with PBS, immunofluorescence staining was visualized under an Olympus fluorescence inverted microscope IX (B&B Microscopes, Ltd., Pittsburgh, Pa.).

Immunoblot analysis for ITGA7, CDKN3, RACGAP1, and β-actin were as follows. Integrin α7 expression was examined in PC3, DU145, 1573, SK-UT-1, H1299, and H358 cells. First, cells were washed with PBS and lysed by RIPA buffer (50 mM Tris-HCl at pH 7.4, 1% Nonidet P-40, 0.25% sodium deoxycholate, 150 mM NaCl, 1 mM EDTA, 1 mM phenylmethylsulfonyl fluoride, aprotinin at 1 μg/mL, leupeptin at 1 μg/mL, pepstatin at 1 μg/mL, and 1 mM Na₃VO₄). The lysates were sonicated and centrifuged at 12,000 g at 4° C. for 30 minutes to remove the insoluble materials. The proteins were separated by sodium dodecyl sulfate-polyacrylamide gel electrophoresis (SDS-PAGE) in 8.5% polyacrylamide gels, and bands were blotted onto a polyvinylidene difluoride (PVDF) membrane. The membrane was blocked with 5% powdered skim milk in Tris-Tween 20 buffer (0.1 M Tris-HCl and 0.1% Tween-20, pH 7.4) for 1 hour at room temperature, followed by a 2-hour incubation with primary anti-ITGA7 antibodies (1:1000 dilution), anti-CDKN3 antibodies (1:1000 dilution; Abnova), or anti-RACGAP1 antibodies (1:500 dilution; Abcam). The membrane was then washed three times with Tris-Tween 20 buffer and incubated with a horseradish peroxidase-conjugated secondary antibody specific for rabbit (anti-ITGA7, 1:1000 dilution), mouse (anti-CDKN3, 1:1000 dilution), or goat (anti-RACGAP 1, 1:1000 dilution) for 1 hour at room temperature. The protein expression was detected with the ECL system (Amersham, Life Science, Piscataway, N.J.) according to the manufacturer's protocols.

To investigate the possibility that overexpression of integrin α7 could abrogate formation of the α7β1 heterodimer, protein extracts were obtained as described in the previous paragraph. Protein extracts were obtained from PITT1 cells that had been induced with tetracycline to express integrin α7. Next, the extracts were incubated with anti-ITGA7 antibody for 16 hours and then with protein G-Sepharose beads for 3 hours to immunoprecipitate the integrin α7 complex. The complex was washed five times with RIPA buffer, and the bound proteins were eluted from the beads with SDS-PAGE sample buffer. The precipitated complexes were separated by SDS-PAGE, electroblotted to a PVDF membrane, and immunoblotted with anti-integrin β1 antibodies (1:500 dilution, Santa Cruz Biotechnology, Inc.). The membrane was then washed three times with Tris-Tween 20 buffer and incubated with a horseradish peroxidase-conjugated secondary antibody specific for goat antibodies (1:1000 dilution) for 1 hour at room temperature. The co-immunoprecipitated integrin β1 was detected with the ECL system (Amersham Life Science), according to the manufacturer's protocols. FIG. 1C shows co-immunoprecipitation of integrin β1 (ITGB1) and ITGA7 in lysates from tetracycline-induced PITT cells.

### Example 1

### Association Between Mutations in the Integrin α7 Sequence with Various Human Malignancies

To investigate whether qualitative alterations in the integrin α7 gene occur in human cancers, integrin α7 genomic DNA and cDNA was sequenced from 66 human cancer specimens (including 28 prostate cancer, 24 hepatocellular carcinomas, six glioblastoma multiforme, and four leiomyosarcoma specimens) and cell lines (including PC3, Du145, and LNCaP cells derived from prostate cancers and SK-UT-1 cells derived from a leiomyosarcoma). In addition, integrin α7 genomic DNA and cDNA from 56 specimens from matched non-tumor tissues were sequenced.

The prostate cancer specimens that were analyzed had been archived as frozen or formalin-fixed paraffin-embedded specimens of tissues from radical prostatectomies from 1985 through 2000. Specimens were selected largely on the basis of their availability or whether sufficient amounts of tumor tissues were present. The ages of patients at the time of surgery ranged from 45 through 79 years. In total, 435 samples were collected. Two hundred ninety-four of the 435 corresponding patients were followed clinically for at least 5 years. Hepatocellular carcinoma specimens were analyzed that had been archived as frozen specimens of liver tissue resections from 1997 through 2002. In total, 24 specimens were collected, and the corresponding patients were followed clinically for at least 5 years. Soft tissue leimyosarcomas were analyzed that had been archived as frozen or formalin-fixed paraffin-embedded specimens of tumor tissue resections from 1970 through 2000. One hundred eleven samples were collected. Sixty-four of the 111 corresponding patients were followed clinically for at least 5 years. Six glioblastoma multiforme specimens were analyzed that had been archived as frozen specimens of tissue resections from 1998 through 2002. Four separate study protocols, all of which included informed consent exemptions, were approved by institutional review board.

Pure tumor specimens were obtained by dissecting freshly resected tissues, typically within 30 minutes of removal from patients. These tissues were frozen at −80° C. and were selected on the basis of tissue availability. Tissues were retrieved and microdissected immediately before the extraction of DNA or total RNA. Tumor cells were microdissected from frozen sections on slides by board-certified pathologists. For matched normal samples, different tissue lineages from the tumor or blood cells (e.g., fat, blood vessels, and seminal vesicles) were obtained. Protocols for tissue banking (which was used for pathology), de-identification, and processing (for molecular analyses) were approved by the institutional review board. The study protocols were exempted from informed consent.

Genomic DNA and total RNA were extracted from various tissues (i.e., prostate, liver, leiomyosarcoma, and glioblastoma multiforme) by use of a QiAmp blood kit and an RNeasy kit from Qiagen, Inc. (Valencia, Calif.), respectively, according to the manufacturer's instructions. Five micrograms of total RNA was used for first-strand cDNA synthesis with d(T)₂₄ primer and Superscript™ II reverse transcriptase (200 U; GIBCO-BRL, Rockville, Md.). Second-strand cDNA synthesis was carried out at 16° C. by adding Escherichia coli DNA ligase (10 U), E. coli DNA polymerase I (40 U), and RNAse H (2 U) to the reaction mixture. T4 DNA polymerase (10 U in 20 μL) was added to blunt the ends of newly synthesized cDNA, and the cDNA was purified by phenol-chloroform extraction and ethanol precipitation. Purified genomic DNA or cDNA from various tissues served as templates for polymerase chain reactions (PCRs) that used a total of 31 sets of primers (Table 1) corresponding to the 27 exons of integrin α7.

TABLE 1
Sequences of genomic and cDNA primers

| | |
|---|---|
| Name | Sequence |

Genome primers

| | |
|---|---|
| ITGa7e1a | TGCGGCTGCTGTAGTTGTCC |
| | (SEQ ID NO: 2) |
| ITGa7e1b | AAGGTAGCAAATCCCGGAGGC |
| | (SEQ ID NO: 3) |
| ITGa7e1c | GCCTCCGGGATTTGCTACCTT |
| | (SEQ ID NO: 4) |
| ITGa7e1d | ATGAGGAGGCCCACAGAGTGG |
| | (SEQ ID NO: 5) |
| ITGa7e2a | TGACCTCTAACTCCTGTCCCTG |
| | (SEQ ID NO: 6) |
| ITGa7e2b | TCTGTTCATGCAGGGCCACAC |
| | (SEQ ID NO: 7) |
| ITGa7e3a | CCTAATTCCCAGTGTCCTGCC |
| | (SEQ ID NO: 8) |
| ITGa7e3b | CCCCATCCGTGCATTCAGTCA |
| | (SEQ ID NO: 9) |
| ITGa7e4a | CCTGGCCCACAGAGTGAAATG |
| | (SEQ ID NO: 10) |
| ITGa7e4b | TCCCCACCATCCAACTCATCC |
| | (SEQ ID NO: 11) |
| ITGa7e4c | GGATGAGTTGGATGGTGGGGA |
| | (SEQ ID NO: 12) |
| ITGa7e4d | GAGGTTTTGGTCCCCTTCTCC |
| | (SEQ ID NO: 13) |
| ITGa7e5a | TACTCTGGATGTCCCCTCCCT |
| | (SEQ ID NO: 14) |
| ITGa7e5b | TCCAGGAGGTGGGAGCTTACA |
| | (SEQ ID NO: 15) |
| ITGa7e6a | TAGGGGTAAGTCACCCTTCCC |
| | (SEQ ID NO: 16) |
| ITGa7e6b | CCTCTACCCACTCACCCATCA |
| | (SEQ ID NO: 17) |
| ITGa7e7a | GGGAGGACCCACACTGAATGT |
| | (SEQ ID NO: 18) |
| ITGa7e7b | CTTTCCAGTTCCCCGTCACAC |
| | (SEQ ID NO: 19) |
| ITGa7e8a | GTGACTGCCTTTTCCCTGTGC |
| | (SEQ ID NO: 20) |
| ITGa7e8b | GATTCCACCCACACCCATTCC |
| | (SEQ ID NO: 21) |
| ITGa7e9a | GAGGCTGACAGCTGGTTCTCT |
| | (SEQ ID NO: 22) |
| ITGa7e9b | GGAAAAGGTTGAGAGGGGCTC |
| | (SEQ ID NO: 23) |
| ITGa7e10a | GTGCTCTTGACTCCCCAATCC |
| | (SEQ ID NO: 24) |
| ITGa7e10b | AAGGATCAAAGGGAGGGCAGG |
| | (SEQ ID NO: 25) |
| ITGa7e11a | TTGGCTCAGGAGCCACCTTTG |
| | (SEQ ID NO: 26) |
| ITGa7e11b | AAACCCAAAAGGGCGAGCCAC |
| | (SEQ ID NO: 27) |
| ITGa7e12a | CCTCCTTTCCCAACATGCCAC |
| | (SEQ ID NO: 28) |
| ITGa7e12b | AAGCCAAGGGGTCAGTGTCCA |
| | (SEQ ID NO: 29) |
| ITGa7e13a | CTGGGGATTGTTCCAGTGAGG |
| | (SEQ ID NO: 30) |
| ITGa7e13b | GGGCTAAACCAGAACCCATGC |
| | (SEQ ID NO: 31) |
| ITGa7e14a | CCCTAGGAATGCCCCTTATCTC |
| | (SEQ ID NO: 32) |
| ITGa7e14b | CTTGAACTCTTGCCCTCCCAC |
| | (SEQ ID NO: 33) |
| ITGa7e15a | TAGCAGGAGTGGGGTCTGACT |
| | (SEQ ID NO: 34) |
| ITGa7e15b | TCAAGACCCCACCCCATCCT |
| | (SEQ ID NO: 35) |
| ITGa7e16a | CCTTGCCTTCTCTCCCATTCC |
| | (SEQ ID NO: 36) |
| ITGa7e16b | AGGGATAAGGGCAGATGTGCC |
| | (SEQ ID NO: 37) |
| ITGa7e17a | TAGACCACCCCTGACTCTA |
| | (SEQ ID NO: 38) |
| ITGa7e17b | TATGACTACCCCCACCTCACC |
| | (SEQ ID NO: 39) |
| ITGa7e18a | ATACTTGCCCCTGCCCACTCA |
| | (SEQ ID NO: 40) |
| ITGa7e18b | GGAAATGTCAATGCCCCCTCC |
| | (SEQ ID NO: 41) |
| ITGa7e19a | GACCTTCTCACCCCTGTTCTG |
| | (SEQ ID NO: 42) |
| ITGa7e19b | GGGCCTCATCCCTGACACTT |
| | (SEQ ID NO: 43) |
| ITGa7e20a | GGTCTCTCCCCTCATACTCTC |
| | (SEQ ID NO: 44) |
| ITGa7e20b | TGTCCCCACATCTAACCCCCA |
| | (SEQ ID NO: 45) |
| ITGa7e21a | GCTGTGATTGGAGGGACACTC |
| | (SEQ ID NO: 46) |
| ITGa7e21b | TCTGGCTGCACCGAGTCTGG |
| | (SEQ ID NO: 47) |
| ITGa7e22a | AGTGGCTTAGACCCCTGTCTG |
| | (SEQ ID NO: 48) |
| ITGa7e22b | CTAGAGCCGAGTGGTATCCTC |
| | (SEQ ID NO: 49) |
| ITGa7e23a | AAGGGTCTCCTTCCCTGTTCC |
| | (SEQ ID NO: 50) |
| ITGa7e23b | ACCTATCCCCCAACCCTGCA |
| | (SEQ ID NO: 51) |
| ITGa7e24a | TGCTCCATTGACCCCTTGCTC |
| | (SEQ ID NO: 52) |
| ITGa7e24b | TGCTCACCCAACCAGGAAGTC |
| | (SEQ ID NO: 53) |
| ITGa7e25a | GCTCTTCAGGCTCCTCATGGT |
| | (SEQ ID NO: 54) |
| ITGa7e25b | TCAGGATGGTGCCCGTCTTCT |
| | (SEQ ID NO: 55) |
| ITGa7e25c | AGAAGACGGGCACCATCCTGA |
| | (SEQ ID NO: 56) |
| ITGa7e25d | TCTTGATGCGACACCAGCAGC |
| | (SEQ ID NO: 57) |
| ITGa7e25e | GCTGCTGGTGTCGCATCAAGA |
| | (SEQ ID NO: 58) |
| ITGa7e25f | CTTGGGGTCCTGTTACACAGG |
| | (SEQ ID NO: 59) |
| ITGa7e26a | CCTGTGTAACAGGACCCCAAG |
| | (SEQ ID NO: 60) |
| ITGa7e26b | GCAAGACTCAAAGAGGCAGAGG |
| | (SEQ ID NO: 61) |
| ITGa7ealta | GCACTAACAGGTCTGTCCTTG |
| | (SEQ ID NO: 62) |
| ITGa7ealtb | AGAGGGTTAGAGCAGTTCTGG |
| | (SEQ ID NO: 63) |
| cDNA primers | |
| ITGA 1 | GATTTCCCTTGCATTCGCTGGG/ |
| | (SEQ ID NO: 64) |
| ITGA 5 | TGCCCTGCTGGCAGAACCCAAATT |
| | (SEQ ID NO: 65) |
| ITGA 6 | GAGGGACGCCCCCAAGGCCATGA/ |
| | (SEQ ID NO: 66) |
| ITGA 7 | GGAAAGCCATCTTGGTTGAGGTCC |
| | (SEQ ID NO: 67) |
| ITGA 8 | TGACTCCATGTTCGGGATCAGCCT/ |
| | (SEQ ID NO: 68) |
| ITGA 4 | GGACAAGGTCACTACAATGGCC |
| | (SEQ ID NO: 69) |
| ITGA 3 | TGTGGAGACGCCATGTTCCAGC/ |
| | (SEQ ID NO: 70) |
| ITGA 9 | CTCAATGCTGATCCCGGAGGTGC |
| | (SEQ ID NO: 71) |
| ITGA 10 | CCCAGGTCACCTTCTACCTCATCC/ |
| | (SEQ ID NO: 72) |
| ITGA 11 | CTGTAGAGTGGGCAGCTGAACACC |
| | (SEQ ID NO: 73) |
| ITGA 12 | GGCCAGTGTCCTCTGCTGAGAAGA/ |
| | (SEQ ID NO: 74) |
| ITGA 2 | CAGGCTGGGACATGGGAACCTA |
| | (SEQ ID NO: 75) |

Each PCR product was gel purified by use of the Geneclean purification kit (Qbiogene, Irvine, Calif.) and then sequenced by use of the corresponding primers as described below. For cDNA sequencing, purified total RNA from various tissues was reversed transcribed with random hexamers (Yu Y P, et al. J Clin Oncol 2004; 22(14):2790-9) for double-stranded cDNA synthesis.

PCR mixtures contained the cDNA templates and six sets of primers (Table 1) distributed along the entire integrin α7 coding region. Automated sequencing of all PCR products used 500 ng of DNA and the BigDye terminator 1.1 cycle sequencing kit (ABI, Foster City, Calif.), as described by the manufacturer. The fluorescence-labeled PCR products were separated by electrophoresis in 6% polyacrylamide gels and analyzed with an ABI Prism 377 DNA sequencer.

When a mutation was identified in a genomic sample, cDNAs were prepared from the corresponding tissue, and the entire integrin α7 coding region was sequenced as described above. Mutations in alleles were determined by clonal sequencing of PCR products (cDNA or genome DNA) by use of primers encompassing the region of both mutations. Loss of heterogeneity was determined by comparing single-nucleotide polymorphisms in the introns or exons of integrin α7 between matched normal and tumor samples.

For reverse transcription PCR (RT-PCR) analysis of integrin α7 expression, the cDNAs from 16 cell lines (including PC3, Du145, LNCaP, H23, H522, H358, H1299, SK-UT-1, Hep3G, 1573, SW-33, SW39, SW40, SW61, SW94, and SW95) were synthesized as described above. The cDNAs from 20 organs (including bone marrow, cerebellum, fetal brain, fetal liver, heart, kidney, lung, placenta, prostate, salivary gland, skeletal muscle, spleen, testis, thyroid, trachea, uterus, colon, small intestine, spinal cord, and stomach) were obtained from Clontech (Mountain View, Calif.). PCRs were performed with primers specific for integrin α7 (Table 1).

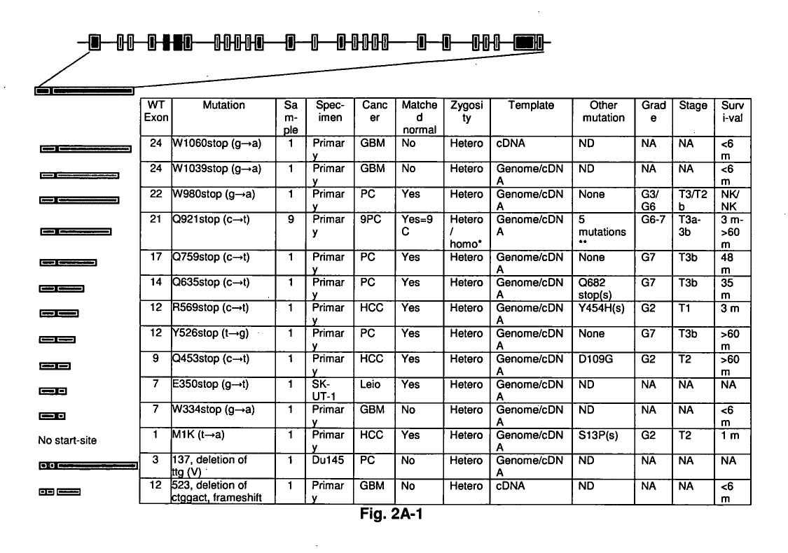

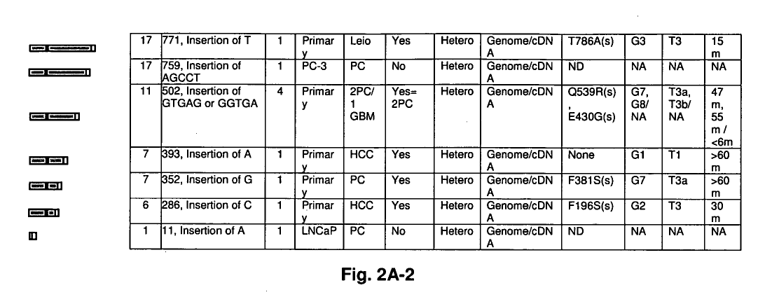

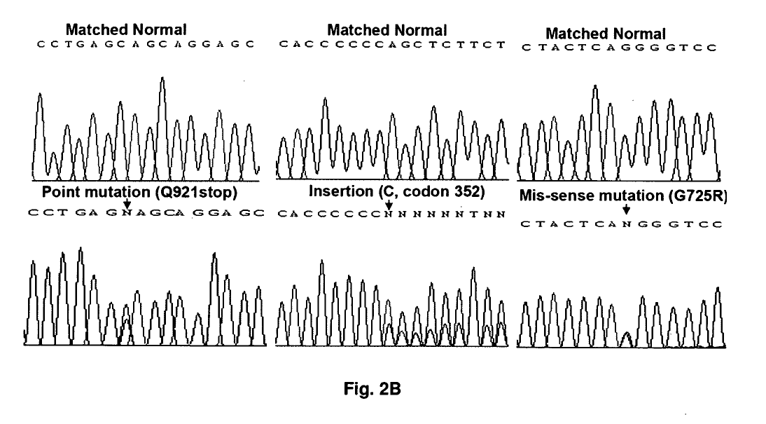

Two types of alterations in the integrin α7 sequence were associated with human malignancies: changes in the amino acid sequence caused by missense mutations and protein truncations caused by nonsense, deletion, or insertion mutations. FIGS. 2A and 2B show the mutations of integrin α7 in human cancer tissues. FIG. 2A shows each structural alteration with an exon number, a description of the mutation (amino acid and nucleotide), number of sample(s) examined, specimen source, type of malignancy, whether matched normal sample was sequenced (for prostate cancer, hepatocellular carcinoma cancer, glioblastoma multiforme, and leiomyosarcoma), zygosity, template used for sequencing, other mutations present in the same samples, pathologic grade, tumor stage, and length of relapse-free survival. FIG. 2B shows a histogram of integrin α7 mutations.

Integrin α7 contains only 50 amino acid residues in its C-terminal cytoplasmic domain, truncations in this domain should adversely affect in its signal transduction ability and other functions. Because truncation mutations have the strongest impact on the structure of the protein, we focused our analysis on such mutations. Truncation mutations of integrin α7 occurred at high frequency in samples from human malignancies (Table 2). All mutations are tabulated as number of samples containing missense, and/or termination mutations.

TABLE 2
Mutation frequency of integrin α7 in primary malignancies*

| | | Termination or | | | | | |
|---|---|---|---|---|---|---|---|
| | | frameshift mutations | | Missense mutations† | | All mutations | |
| | | | Frequency, % | | Frequency, % | | Frequency, % |
| | | No. | (95% CI) | No. | (95 CI %) | No. | (95% CI) |
| Prostate cancer | 28 | 16 | 57 | 13 | 46 | 20 | 71 |
| | | | (37 to 76) | | (28 to 65) | | (55 to 85) |
| Hepatocellular | 24 | 5 | 21 | 7 | 29 | 8 | 33 |
| carcinoma | | | (7 to 42) | | (12 to 46) | | (16 to 51) |
| Glioblastoma | 6 | 5 | 83 | NA | NA | 5 | 83 |
| multiforme | | | (36 to 99) | | | | (36 to 99) |
| Leiomyosarcoma | 4 | 1 | 25 | 1 | 25 | 1 | 25 |
| | | | (0.6 to 81) | | (0.6 to 81) | | (0.6 to 81) |

*CI = confidence interval;
NA = not available;
†Missense mutation in sequence as compared with matched normal samples; all mutations are tabulated as number of samples containing missense and/or termination mutations.

In the prostate cancer specimens, the rate of integrin α7 mutations was 57% (95% CI=37% to 76%; i.e., 16 mutations in 28 specimens). In glioblastoma multiforme specimens, the rate reached 83% (95% CI=36% to 99%; i.e., five mutations in six specimens). In leiomyosarcoma specimens, the rate was 25% (95% CI=0.6% to 815; i.e., one mutation in four specimens). In hepatocellular carcinoma specimens, the rate was 21% (95% CI=7% to 42%; i.e., five in 24 specimens). All of these mutations had major structural consequences as indicated in FIG. 2A, including protein truncation because of a premature stop codon, a frameshift because of deletions or insertions of nucleotides, or loss of the translational start site.

The integrin α7 mutations were spread across the coding region, but a hot spot (n=9 specimens) was identified in codon 921, in which a glutamine codon was mutated to a stop codon. Fourteen samples contained both truncation and missense mutations, 10 of which were identified as mutations in separate alleles.

Table 3 shows the association between integrin α7 mutations with various pathologic and clinical factors. Prostate cancers with integrin α7 mutations, compared with those without such a mutation, were generally less differentiated (Fisher's exact test, P=0.009), had a more advanced stage (P=0.005), and were more likely to be associated with relapse (nine recurrences among 13 patients with integrin α7 mutations vs. one among eight without such mutations; odd ratio [OR]=14, 95% CI=1.15 to 782, P=0.024). However, hepatocellular carcinomas with integrin α7 mutations were only associated with shorter relapse-free survival than tumors without such mutations (five recurrences among eight patients with integrin α7 mutations vs. one among 16 without such mutations, OR=21, 95% CI=1.6 to 1245; P=0.007) (Table 3).

TABLE 3
Pathologic and clinical factors and integrin α7 mutations*

| | | | | | Hepatocellular | | | | Glioblastoma | | | | | |
|---|---|---|---|---|---|---|---|---|---|---|---|---|---|---|
| | Prostate cancer† | | | | carcinoma† | | | | multiforme†‡ | | | Leiomyosarcoma†‡ | | |
| | Yes | No | OR (95% CI) | P | Yes | No | OR (95% CI) | P | Yes | No | P | Yes | No | P |
| Poor | 18/20 | 3/8 | 13 | 0.009 | 1/8 | 1/16 | 2.1 | NS | 5/5 | 1/1 | NS | NA | NA | NA |
| differentiation§ | | | (1.4 to 200) | | | | (0.2 to 179) | | | | | | | |
| Advanced | 17/20 | 2/8 | 15 | 0.005 | 3/8 | 5/16 | 1.3 | NS | NA | NA | NA | 1/1 | 1/3 | NS |
| stage# | | | (1.7 to 219) | | | | (0.14 to 11) | | | | | | | |
| <5 years of | 9/13 | 1/8 | 14 | 0.024 | 5/8 | 1/16 | 21 | .007 | 5/5 | 1/1 | NS | 1/1 | 1/3 | NS |
| relapse-free | | | (1.15 to 782) | | | | (1.6 to 1245) | | | | | | | |
| survival¶ | | | | | | | | | | | | | | |

OR = odds ratio; CI = confidence interval; NA = not available; NS = not statistically significant. All statistical tests were two sided. Fisher's exact tests were used.
†Number with factor/total number in group. “Yes” indicates samples with mutation, where “No” indicates samples without a mutation.
‡Odds ratio and 95% confidence intervals were not available for these cancer types.
§Combined Gleason's scores 7 or above for prostate cancer or grade 3 or above for hepatocellular carcinoma and leiomyosarcoma
#T3a or above for prostate cancer or T3 or above for hepatocellular carcinoma and leiomyosarcoma
¶Only samples with at least 5 years of clinical follow-up were analyzed.

### Example 2

### Association Between Integrin α7 Expression with Metastasis and Relapse of Human Malignancies

Meta-analysis of microarray data on integrin α7 expression was performed to correlate integrin α7 expression with metastasis in one of two types of human cancers: prostate cancer and leiomyosarcoma. A PubMed search was conducted to identify articles containing Affymetrix data sets on human leiomyosarcoma or prostate cancer using search terms “Affymetrix,” “primary prostate cancer,” and “primary leiomyosarcoma.” Seven relevant articles about prostate cancer and one for human soft tissue leiomyosarcoma were found. Four sets of data from these eight articles were selected because of their availability. Among them, three were from the University of Pittsburgh and one was from Memorial Sloan-Kettering. For meta-analysis, Affymetrix CEL files of all samples from the four articles (LaTulippe E, et al. Cancer Res 2002; 62(15):4499-506; Luo J H, et al. Mol Carcinog 2002; 33(1):25-35; Ren B, et al. Oncogene 2006; 25(7):1090-8; Yu Y P, et al. J Clin Oncol 2004; 22(14):2790-9) were re-analyzed with GCOS version 1.0 and normalized to an average target intensity of 500 for each sample.

The results were exported to Microsoft Excel for statistical analysis. Fold changes of integrin α7 in tumor samples were calculated as average intensity of tumor samples over average of the normal controls in the same set of data. Two-sided Student's t tests were performed to obtain P values. Confidence intervals were calculated as described above. The number of samples analyzed from each article was as follows: 156 samples from Yu et al. (Yu Y P, et al. J Clin Oncol 2004; 22(14):2790-9), 30 from Luo et al. (Luo J H, et al. Mol Carcinog 2002; 33(1):25-35), 26 from LaTulippe et al. (LaTulippe E, et al. Cancer Res 2002; 62(15):4499-506), and 29 from Ren et al. (Ren B, et al. Oncogene 2006; 25(7):1090-8). These are the sources indicated in Table 4.

TABLE 4
Meta-analysis of integrin α7 expression in cancer tissues*

| | Non-relapse tumors | | Relapse tumors | | |
|---|---|---|---|---|---|
| | Fold (95% CI) | P value | Fold (95% CI) | P value | Source |
| Prostate cancer (2002) | −2.7 | 0.002 | −6.1 | <0.001 | Luo |
| | (−2.33 to −3.07) | | (−5.97 to −6.23) | | et al. |
| Prostate cancer (2003) | −4.5 | <0.001 | −5.3 | <0.001 | La |
| | (−4.48 to −4.52) | | (−5.28 to −5.32) | | Tulippe |
| | | | | | et al. |
| STL (2003) | −1.1 | >0.05 | −41.1 | 0.01 | Ren |
| | (−1.75 to 1.55) | | (−37.3 to −44.83) | | et al. |
| Prostate cancer (2004) | −2.9 | 0.002 | −4.4 | <0.001 | Yu |
| | (−2.87 to −2.93) | | (−4.18 to −4.62) | | et al. |

*STL = soft tissue leiomyosarcoma; CI = confidence interval. The statistical test used was a two-sided Student's t test. Data are the fold change of average in arbitrary units of tumors over corresponding normal tissues. The individual CEL file of each sample was normalized to target intensity of 500 arbitrary units in GCOS1.0 ™ from Affymetrix, Inc., and exported to a Microsoft Excel spreadsheet for statistical analysis.

Meta-analysis of microarray data on integrin α7 expression found low integrin α7 expression (2.7-fold decreased to 4.5-fold decreased expression) in prostate cancers from Memorial Sloan-Kettering Cancer Institute and University of Pittsburgh, that did not metastasize but even lower expression (4.4-fold decreased to 6.1-fold decreased expression) in those that metastasized, when compared with normal prostate (4325 units in average) (Table 4).

Soft tissue leiomyosarcomas that did not metastasize and normal smooth muscle tissue had approximately the same level of integrin α7 expression, but integrin α7 expression in highly aggressive soft tissue leiomyosarcomas was decreased by 41.1-fold (95% CI=37.4-fold to 44.8-fold), compared with normal smooth muscle (Ren B, et al. Gene expression analysis of human soft tissue leiomyosarcomas. Hum Pathol 2003; 34(6):549-58). RT-PCR analyses of 20 human organs and 16 cell lines derived from tumors of prostate, brain, liver, smooth muscle, lung, and kidney detected expression of integrin α7 mRNAs in all tissues and cell lines examined.

Human tissue samples were immunostained to determine whether integrin α7 protein expression was decreased in prostate cancer and leiomyosarcoma, compared with normal tissues. For tissue microarray analysis, 701 formalin-fixed and paraffin-embedded prostate tissue specimens (407 from prostate cancer tissue and 294 from normal prostate tissue as described above) were arrayed onto six slides, with one or two samples from each specimen (Ren B, et al. MCM7 amplification and overexpression are associated with prostate cancer progression. Oncogene 2006; 25(7):1090-8). Patients in this group ranged in age from 45 to 79 years, and complete 5-year follow-up data was available for 266 patients with prostate cancer (University of Pittsburgh Medical Center tissue collection archive, 1985 through 2000). Tissue array slides and thin section of paraffin-embedded tissues were used to study soft tissue leiomyosarcoma specimens (34 normal tissue samples and 107 leiomyosarcomas, including samples from 60 patients with more than 5 years of follow-up). These specimens were arrayed onto three slides, with two samples from each specimen.

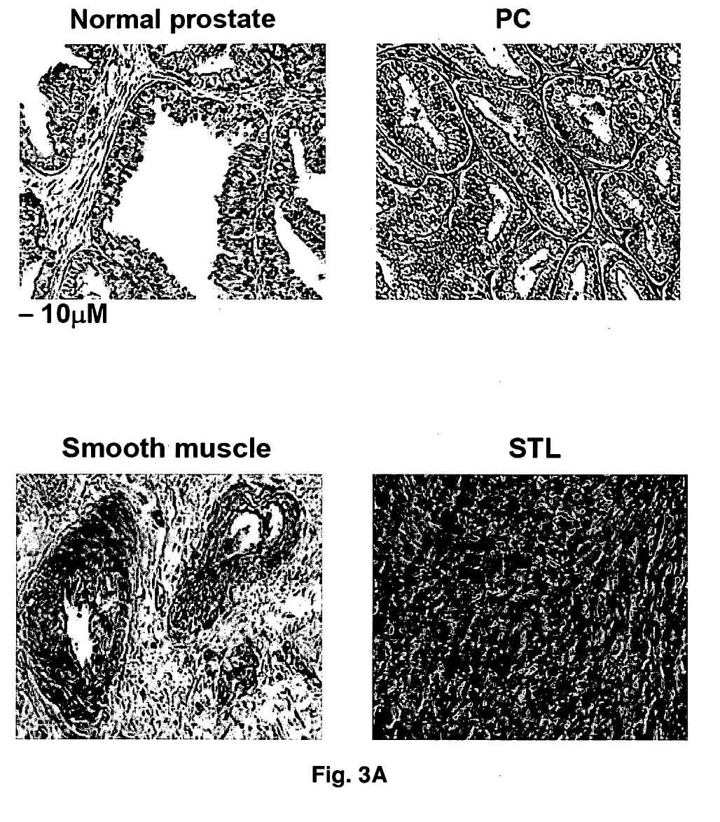

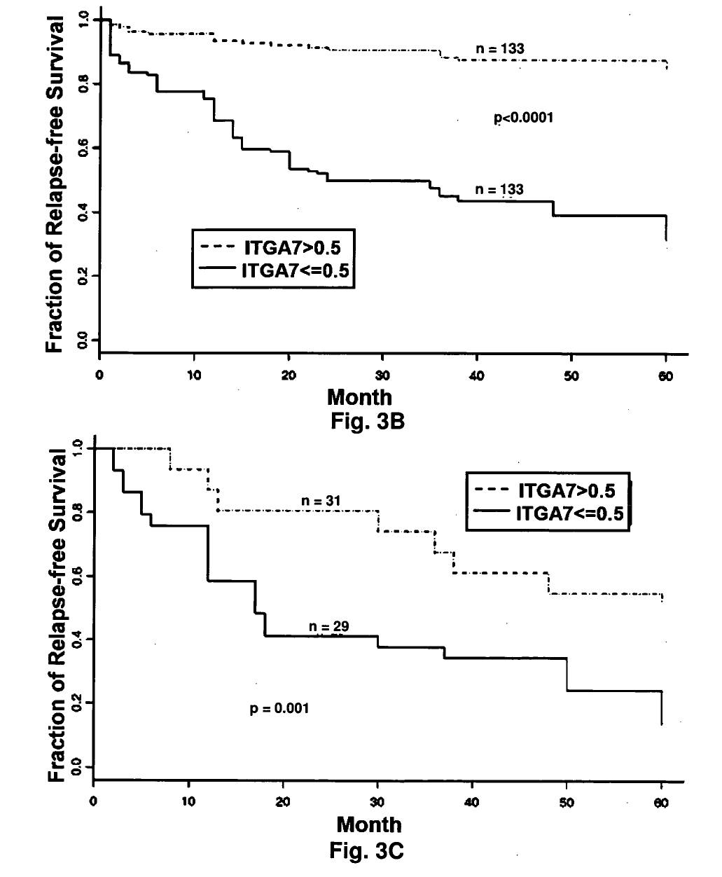

Immunohistochemistry was performed with purified integrin α7 peptide antiserum (1:1000 dilution), as described above. The peptide antibody was omitted in negative controls. The sections were then incubated with horseradish peroxidase-conjugated anti-rabbit IgG for 30 minutes at room temperature. Slides were exposed to a 3,3′-diaminobenzidine solution to visualize immunostaining. Integrin α7 immunostaining was graded on a scale of 0-3 as follows: 0=no expression; 0.5=focal positive; 1=weak; 2=moderate; 3=strong. A threshold of score of 0.5 was used in the presentation to determine the likelihood of tumor relapse, and it was chosen so that two groups had balanced sample sizes. Moving the threshold to 0 or 1 resulted in a similar conclusion. FIG. 3A shows photomicrographs of immunohistochemically-stained tissue from a normal prostate, prostate cancer, smooth muscle, and leiomyosarcoma.

Immunostaining of prostate tissues showed that normal prostate gland tissue had a moderate level of integrin α7 expression, with an average score of 1.82 (95% CI=1.74 to 1.89). The acinar cells were more intensely stained than the basal cells. In contrast, many prostate cancer tissues had no integrin α7 or only focal positive staining for integrin α7, with an average score of 0.740 (95% CI=0.699 to 0.789, P<0.001) (Table 5, FIG. 3A). A further decrease in the level of integrin α7 expression was observed in metastasizing prostate cancer tumors, with an average score of 0.414 (95% CI=0.348 to 0.480) (Table 5).

TABLE 5
Immunostaining of integrin α7 in human
prostate cancer and leiomyosarcoma samples

| | | Average score* (95% |
|---|---|---|
| Tissue | No. of samples | confidence interval) |
| Benign Prostate | 294 | 1.82 (1.74 to 1.89) |

Prostate Cancer

| | | |
|---|---|---|
| All | 407 | 0.74 (0.70 to 0.79) |
| Non-relapse | 155 | 0.93 (0.86 to 1.00) |
| Relapse | 111 | 0.41 (0.35 to 0.48) |
| Benign smooth muscle | 34 | 1.43 (1.24 to 1.61) |

Leiomyosarcoma

| | | |
|---|---|---|
| All | 107 | 0.65 (0.55 to 0.75) |
| Non-relapse | 20 | 1.13 (0.88 to 1.37) |
| Relapse | 40 | 0.63 (0.46 to 0.79) |

A scale of 0-3 as follows: 0 = no expression; 0.5 = focal positive; 1 = weak; 2 = moderate; 3 = strong.

Strong integrin α7 expression was identified in smooth muscle tissue surrounding small vessels. Soft tissue leiomyosarcoma tissue from patients with a relatively mild clinical course (i.e., tumor-free survival of patients was >5 years) had slightly lower integrin α7 expression (average score=1.125, 95% CI=0.876 to 1.373) than normal smooth muscle (1.43, 95% CI=1.239 to 1.614). In addition, aggressive soft tissue leiomyosarcoma tissue (from patients with a relapse within 5 years) had much lower integrin α7 expression (0.625, 95% CI=0.456 to 0.794) (Table 5).

FIG. 3B shows the relapse-free survival of patients with prostate cancer, where FIG. 3C shows the relapse-free survival of patients with leiomyosarcoma. The cutpoint used was an integrin α7 score of 0.5 or less versus more than 0.5. Analysis includes only samples with more than 60 months clinical follow-up. P values were from log-rank tests.

In prostate cancer, 66 (95% CI=54 to 78) patients were at risk at 30 months in the ITGA7 group with a score of 0.5 or less and 120 (95% CI=112 to 126) patients were at risk at 30 months in the ITGA7 group with a score of more than 0.5. At the 60 month time point, 42 (95% CI=32 to 53) patients were at risk in the ITGA7 group with a score of 0.5 or less and 113 (95% CI=103 to 120) patients were at risk in the ITGA7 group with a score of more than 0.5 group.

In leiomyosarcoma, 11 (95% CI=6 to 17) patients were at risk at 30 months in the ITGA7 group with a score of 0.5 or less and 23 (95% CI=17 to 27) patients were at risk at 30 months in the ITGA7 group with a score of more than 0.5. At the 60 month time point, 4 (95% CI=1 to 9) patients were at risk in the ITGA7 group with a score of 0.5 or less and 16 (95% CI=10 to 22) patients were at risk in the ITGA group with a score of more than 0.5. All statistical tests were two-sided.

Among patients contributing with prostate cancer or leiomyosarcoma samples, statistically significant decreases in 5-year metastasis-free survival were associated with little or no expression of integrin α7 in tumors, compared with at least weak expression of integrin α7 in tumors (for example, among patients with prostate cancer, 5-year survival rate associated with tumors with focal or no integrin α7 expression was 32%, 95% CI=24.4% to 40.3%, and that associated with higher integrin α7 expression was 85%, 95% CI=79.0% to 91.0%; P<0.001). These results support a role of integrin α7 in cancer metastasis and indicate that integrin α7 may have a role in cancer behavior.

### Example 3

### Association of Integrin α7 Expression with Tumorigenesis and Metastasis in Cell-Based Assays

To examine the effect of alterations in the level of integrin α7 mutations on tumorigenesis (as assessed by colony formation and growth in soft agar), we increased the level of integrin α7 in the deficient cell lines (i.e., PC3, Du145 and SK-UT-1) to normal wild-type levels by use of an integrin α7 expression vector (pCMV-integrin α7 vector) or decreased its level by 70% by use of siRNA against integrin α7.

PC-3 cells contains a frameshift mutation at codon 759 in one integrin α7 allele, and Du145 cells contains a two-amino acid deletion mutation in integrin α7. SK-UT-1 cells had a premature stop codon at position 350 in one integrin α7 allele, so that integrin α7 protein was expressed only from the remaining non-mutated allele. Cell lines H1299 and H358 expressed normal wild-type levels of integrin α7 and lacked integrin α7 mutations.

To construct the inducible integrin α7 expression vector pcDNA4-ITGA7, full-length integrin α7 cDNA was ligated at the NotI and KpnI sites of pcDNA4/TO/MYC-HIS-B (Invitrogen, CA). This plasmid was then co-transfected into PC-3 cells with pcDNA6/TR, which encodes the tetracycline repressor. Transfected cells were selected by use of zeomycin (pcDNA4/TO/MYC/HIS-B-transfected cells) and blasticidin S (pcDNA6/TR-transfected cells) (Invitrogen). Selected clonal cell lines, including two that were designated PITT1 and PITT2, were tested for doxycycline inducibility (1 μg/mL) by western blot analysis with antibodies specific for integrin α7 or β-actin (the loading control). As shown in FIG. 1B, PITT1 cells were also tested by immunofluorescence analysis. FIG. 1C shows co-immunoprecipitations using anti-integrin α7 antibodies indicated that integrin α7 and integrin β1 formed a protein complex because immunoprecipitates contained integrin β1 protein.

Integrin α7 cDNA was generated from total RNA from normal donor prostate tissue by extended long PCR with primers specific for the 5′ and 3′ ends of integrin α7 (Jing L, et al. Am J Pathol 2004; 164(5):1799-806). The 3.7-kilobase PCR product was ligated into a TA cloning vector (Invitrogen) and from there cloned into a pCMVscript vector (Clontech) with HindIII and XhoI (New England Biolab, Ipswich, Mass.). The final pCMV-ITGA7 construct was sequenced by the automatic sequencing method, as described above, to confirm that no mutations had been introduced. This construct was transfected into Du145, PC-3, or SK-UT-1 cells. Colonies containing pCMV-ITGA7 were selected for with medium that included G418 (400 μg/mL).

To construct the small interfering RNA (siRNA) vectors for CDKN3, RACGAP1, integrin α7, and a scrambled control sequence, oligonucleotides corresponding to the following regions of CDKN3 mRNA (5′-CACCGGAGCTTACAACCTGCCTTAAATTGATATCCGTTT AAGGCAGGTTGTAAGCTC-3′ (SEQ ID NO: 76)/5′-AAAAGAGCTTACAACCTGCCTTAAACGGATATCAATTTAAGGCAGGTTGTAAGCTCC-3′ (SEQ ID NO: 77)), RACGAP1 (5′-CACCGTTTGCACTTTGGATGCT GAAATTGATATCCGTTTCAGCATCCAAAGTGCAAA-3′ (SEQ ID NO: 78)/5′-AAAATTTGCACTTTGGATGCTGAAACGGATATCAATTTCAGCATCCAAAGTGCAAAC-3′ (SEQ ID NO: 79)), integrin α7 (5′-CACCGACTCCCAACCACTGGTTCTCCTTGCCGAAGCAAGGAGAACCAGTGGTTGGGAGT-3′ (SEQ ID NO: 80)/5′-AAAAACTCCCAACCACTGGTTCTCCTTGCTTCGGCAAGGAGAACCAGTGGTTG GGAGTC-3′ (SEQ ID NO: 81)), or scrambled siRNA (5′-CACCGTAATGTATTGGAACGCATATTTTGATATCCGAATATGCGTTCCAATACATTA-3′ (SEQ ID NO: 82)/5′-AAAATAATGTATTGGAACGCATATTCGGATATCAAAATATGCGTTCCAATACATTA-3′ (SEQ ID NO: 83)) were annealed and ligated into a pENTR/U6 vector. The ligated products were transfected into E. coli and plated on kanamycin plates (50 μg/mL). Six colonies per transfection were picked and sequenced for the presence of inserts. The selected clones, which suppress the expression of integrin α7 (ITGA7), CDKN3, or RACGAP1, respectively, were then transfected into cultured cells to generate pENTR-siITGA7-transfected H1299 or H358 cells or pENTR-siCDKN3- and pENTR-siRACGAP1-transfected PITT1 and PITT2 cells.

Colony formation and soft agar anchorage-independent assays were similar to those previously described (Jing L, et al. Am J Pathol 2004; 164(5):1799-806). PC-3, Du145, SK− UT-1 cells that were transfected with pCMVscript or pCMV-integrin α7 and H1299 and H358 cells that were transfected with pENTR-siITGA7 were used.

For colony formation assay, 5000 cells were cultured in 60-mm dishes. Triplicate experiments were performed for each cell clones. Medium was changed every 4 days. On the 10^{th} day, the plates were stained with 1% crystal violet, and colonies with diameter of more than 2 mm were counted.

For the soft agar colony formation assay, the same cell lines were used. In brief, 5000 cells were cultured on a plate containing 2% base agar and 0.43% top agar in the medium described above and incubated at 37° C. for 21 days. Plates were stained with 0.005% Crystal violet for 1 hour. Colonies were counted by use of a dissecting microscope.

For the wound healing assay (Yu Y P, Luo J H. Myopodin-mediated suppression of prostate cancer cell migration involves interaction with zyxin. Cancer Research 2006; 66(15):7414-9), Du145, PC-3, or SK-UT-1 cells were cultured in six-well culture plates in the medium described above. After cells reached confluence, a plastic pipette tip was drawn across the center of the well to produce a clean crevice that was 1 mm wide. Microscopic images of the “wounds” were taken in five different areas for each experiment (at an original magnification of ×10 with an Olympus inverted system microscope IX). After culturing for 24 hours at 37° C. in F12K medium (PC-3 cells) or modified Eagle medium (Du145 and SK-UT-1 cells) containing 10% fetal bovine serum, images of original locations were taken again, and recovered areas (i.e., the bare area into which cells migrated) was measured as a percentage of the original wound.

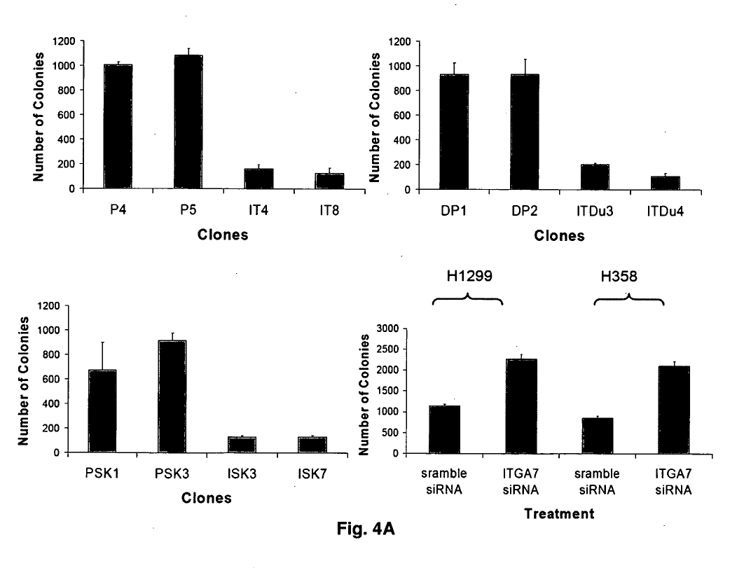

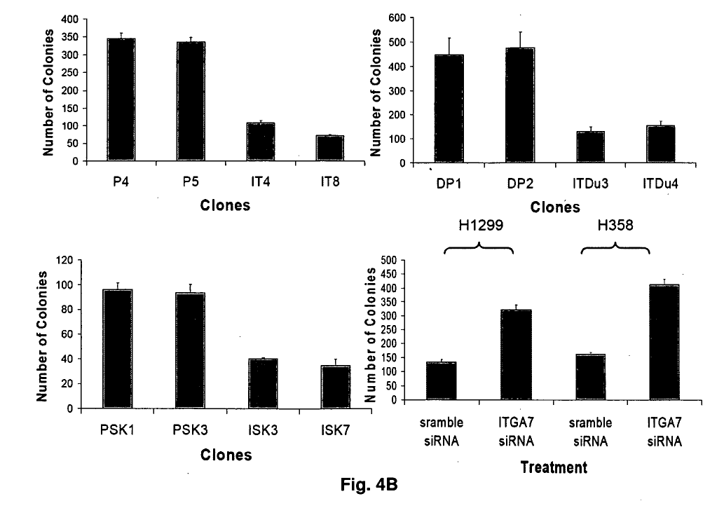

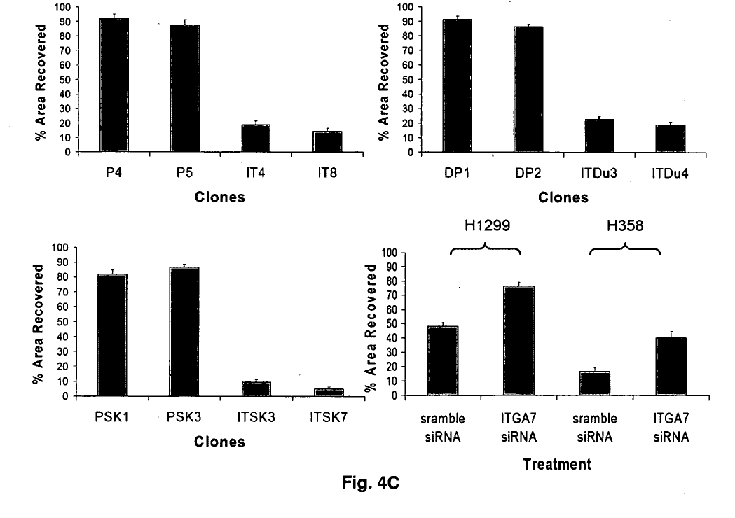

FIG. 4 shows two sets of experiments, where the first set involves cell lines with deficient levels of integrin α7 and the second set involves cell lines with normal levels of integrin α7 expression.

In a first set of experiment, the expression of integrin α7 was increased to normal wild-type levels in PC-3, Du145, and SK-UT-1 cells by transfecting them with an integrin α7 expression vector (pCMV-integrin α7 vector). Then, the ability of these cells to form colonies and grow on soft agar was compared with that of corresponding pCMVscript-transfected control cells.

In a second set of experiments, level of integrin α7 expression in H1299 and H358 cells was decreased by transfecting cells with integrin α7-specific siRNAs or scrambled siRNAs expressing vectors. Then, we investigated the colony formation ability and growth on soft agar of these cells. Both integrin α7-specific siRNA-expressing cell lines formed more colonies and grew better on soft agar than their corresponding scramble control cell lines.

FIG. 4A shows the colony formation analysis of integrin α7-transfected cells. In the colony formation assay, the rate of colony formation was reduced by 7.1-fold (95% CI=4.91-fold to 9.38-fold) in integrin α7-transfected PC-3 cells as compared with pCMVscript-transfected control PC-3 colonies, by 6-fold (95% CI=3.87-fold to 8.13-fold) in integrin α7-transfected Du145 cells as compared with pCMVscript-transfected control Du145 colonies, and by 5.9-fold (95% CI=5.59-fold to 6.28-fold) in integrin α7-transfected SK-UT-1 cells as compared with pCMVscript-transfected control SK-UT-1 colonies.

FIG. 4B shows the soft agar anchorage-independent growth analysis of integrin α7-transfected cells after 22 days. Cells were assayed for their ability to grow in soft agar. In the soft agar growth assay, pCMVscript-transfected control cells formed large colonies with up to 100 cells on soft agar, but integrin α7-transfected cells with higher (normal) levels of integrin α7 expression formed fewer and smaller colonies. Specifically, for PC-3 cells, there was a 3.8-fold (95% CI=3.15-fold to 4.39-fold) reduction in colony formation; for Du145 cells, there was a 3.2-fold (95% CI=2.83-fold to 3.62-fold) reduction; and for SK-UT-1 cells, there was a 2.6-fold (95% CI=2.25-fold to 2.80-fold) reduction.

To investigate the role of integrin α7 in metastasis, we examined the relationship between the level of integrin α7 expression and cell migration by use of wound-healing assays with PC-3, Du145, SK-UT-1, H1299, and H358 cells. FIG. 4C shows the wound-healing analysis of integrin α7-transfected cells. When the expression of integrin α7 was increased in PC-3, Du145, and SK-UT-1 cells with low integrin α7 expression by transfecting cells with integrin α7 expression vectors, the rate of migration, compared with that in corresponding pCMVscript-transfected cells, was reduced by 5.4-fold (95% CI=4.68-fold to 6.19-fold), 4.3-fold (95% CI=3.86-fold to 4.64-fold), and 11.7-fold (95% CI=5.59-fold to 17.85-fold), respectively.

H1299 and H358 cells express a normal level of integrin α7 and have low motility. When these cells were transfected with an integrin α7-specific siRNA to decreased integrin α7 expression, the rate of migration increased by 2-fold (95% CI=1.57-fold to 2.41-fold) compared with that of corresponding scrambled siRNA-transfected control cells. Thus, the level of integrin α7 expression appears to be inversely associated with tumor cell migration.

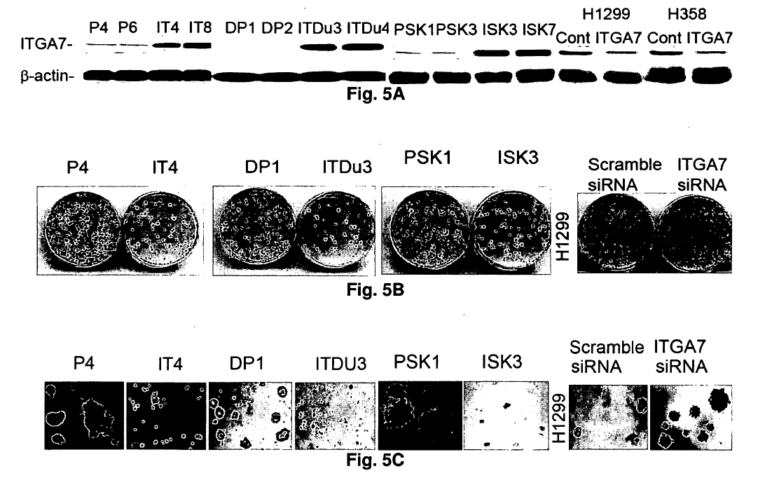

FIG. 5A shows immunoblots of cell lines using rabbit antibodies against integrin α7 (top panel) and mouse monoclonal antibody against β-actin (bottom panel). Immunoblots are shown for cells transfected with pCMVscript or pCMV-integrin α7, including PC-3 cells (P4 and P5=vector control, and IT4 and IT8=ITGA7), Du145 cells (DP1 and DP2=vector control, ITDu3 and ITDu4=ITGA7), and SK-UT-1 cells (PSK1 and PSK3=vector control, and ISK3 and ISK7=ITGA7). H1299 and H358 were transfected with vectors expressing either scramble small interfering RNA (siRNA) or integrin α7 specific siRNA. FIG. 5B shows representative photographs of hematoxylin-stained colonies. FIG. 5B shows representative photomicrographs of colonies formed in 0.4% soft agar 22 days after inoculation.

### Example 4

### Investigating the Tumor-Suppressing Activity of Integrin α7 in an In Vivo Mouse Model

To investigate the tumor suppressor activity of integrin α7, we generated xenograft tumors in severe combined immune deficiency (SCID) mice implanted with siRNA vector-transfected PC-3 and Du145 prostate cancer cells and corresponding cells transfected with integrin α7 expression constructs and then compared the volume of tumors as a function of integrin α7 expression. Clones of integrin α7-expressing PC-3 and Du145 cells and their corresponding controls were assayed for tumor growth in SCID mice within six weeks of tumor cells inoculation.

Approximately 1×10⁷ viable PC-3 and Du145 cells, suspended in 0.2 mL of Hanks' balanced salt solution (Krackeler Scientific, Inc., Albany, N.Y.) were subcutaneously implanted in the abdominal flanks of 48 SCID mice to generate one tumor per mouse. Mice were observed daily, and their body weight, tumor size, and lymph-node enlargement were recorded weekly. Tumor and lymph node size were measured on the diameter. After 6 weeks or when mice became moribund, which ever occurred first, mice were killed, and necropsies were performed. Serial sections of formalin-fixed, paraffin-embedded lung, brain, liver, kidney, vertebra, and lymph node specimens were collected, stained with hematoxylin and eosin, and examined microscopically.

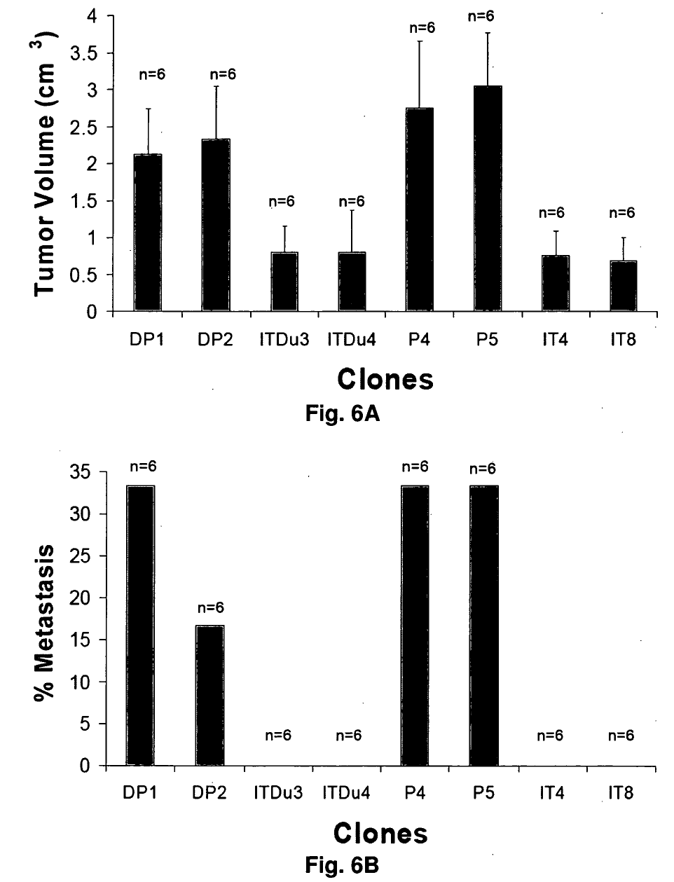

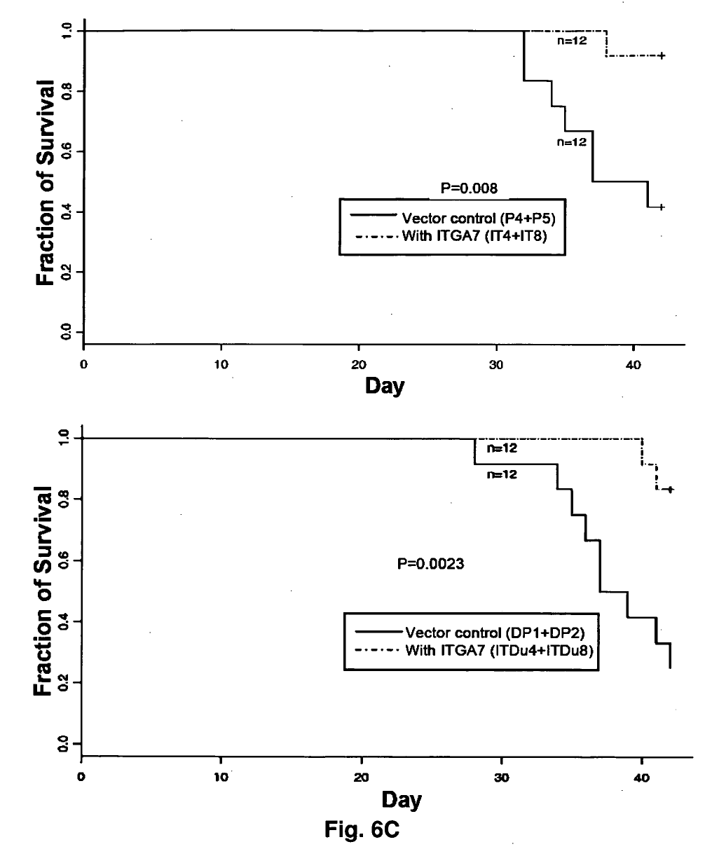

FIG. 6A shows the reduction of tumor volume when integrin α7-expressing tumor cells were implanted in SCID mice. Six weeks after implantation, tumors from integrin α7-transfected Du145 cells had an average volume of 0.8 cm³, and tumors from siRNA vector-transfected Du145 cells had an average volume of 2.2 cm³ (difference=1.4 cm³, 95% CI of difference=0.9 to 2.1, P 0.001). Similarly, 6 weeks after implantation, the volume of tumors from integrin α7-transfected PC-3 cells was 0.7 cm³ and that from siRNA vector-transfected PC-3 cells was 2.9 cm³ (difference=2.2, 95% CI of difference=1.5 to 2.9, P<0.001).

FIG. 6B shows the suppression of metastasis in integrin α7-expressing tumor cells. No visible metastases were identified in mice with integrin α7-transfected Du145 or PC-3 tumors. However, metastasis were observed in three (25%) of the 12 mice with siRNA vector-transfected Du145 tumors and in four (33%) of the 12 mice with vector-transfected PC-3 tumors. For P4, P5, or DPI cells, the rate of metastasis was 2/6 or 33% (95% CI=4% to 78). For DP2 cells, it was ⅙ or 17% (95% CI=0.4% to 64%). For P4 and P5 cells combined, the rate of metastasis was 33% (95% CI=10% to 65%). For IT4 and IT8 cells, it was 0%. For DP1 and DP2 cells, it was 25% (95% CI=5% to 57%). For ITDU3 and ITDU4 cells, it was 0%. The number of mice in each group that died before 42 days was: three of the six mice died for P4 tumors; four of the six for P5 cells; one of the six for IT4 cells; zero of the six for IT8 cells; four of the six for DPI cells; five of the six for DP2 cells; one of the six for ITDU3 cells; and one of the six for ITDU4 cells.

FIG. 6C shows that the 6-week survival of mice bearing integrin α7-transfected Du145 tumors (83%, 95% CI=62% to 100%) or PC-3 tumors (92%, 95% CI=76% to 100%) was higher than that of mice bearing tumors from the corresponding siRNA vector-transfected cells tumors (25%, 95% CI=0.5% to 49.5%, and 42%, 95% CI=13.8% to 69.5%). In PC-3 cells at risk at 37 days, 6 (95% CI=3 to 9) mice in the control-transfected group and 12 (95% CI=9 to 12) mice in the integrin α7-transfected group were at risk. At 42 days, 5 (95% CI=2 to 9) mice were at risk in the control-transfected group and 11 (95% CI=7 to 12) mice were at risk in the integrin α7-transfected group. For Du145 cells at 37 days, 6 (95% CI=3 to 9) mice in the control-transfected group and 12 (95% CI=9 to 12) mice in the integrin α7-transfected group were at risk. At 42 days, 3 (95% CI=1 to 7) mice in the control-transfected group and 10 (95% CI=6 to 12) mice in the integrin α7-transfected group were at risk. All statistical tests were two-sided. Thus, increased integrin α7 was associated with decreased tumor growth and metastasis in vivo.

### Example 5

### Determining the Effect of Integrin α7 on Global Gene Expression

To determine whether the expression of integrin α7 alters the global gene expression profile, we transfected PC-3 and SK-UT-1 cells with a tetracycline-inducible integrin α7 expression vector (pcDNA4-ITGA7) and used microarray analysis to compare gene expression in these cells in the presence of tetracycline with that in un-induced cells. Within 24 hours of integrin α7 induction, the expression of cyclin D kinase inhibitor 3 (CDKN3) and rac GTPase-activating protein 1 (RACGAP1) was also increased.

Total RNA was extracted from un-induced and induced PITT1 cells and purified with Qiagen RNeasy kit (Qiagen). Five micrograms of total RNA was used for first-strand cDNA synthesis with T7-d(T)₂₄ primer having the sequence of GGCCAGTGAATTGTAATACGA CTCACTATAGGGAGGCGG-(dT)₂₄ (SEQ ID NO: 84) and Superscript II reverse transcriptase (200 U; GIBCO-BRL, Rockville, Md.). Second-strand cDNA synthesis was carried out at 16° C. by adding E. coli DNA ligase (10 U), E. coli DNA polymerase 1 (40 U), and RNAse H (2 U) to the reaction mixture. T4 DNA polymerase (10 U in 20 μL) was added to blunt the ends of newly synthesized cDNA, and the cDNA was purified by phenol-chloroform extraction and ethanol precipitation.

Purified cDNAs were then incubated at 37° C. for 4 hours in an in vitro transcription reaction mixture containing 10 mM ATP, 10 mM biotin-CTP, 10 mM GTP, and 10 mM biotin-UTP to produce biotin-labeled complementary RNA (cRNA) by use of the MEGAscript system (Ambion, Inc, Austin, Tex.). cRNA (15-20 μg) was fragmented by incubating in a buffer containing 200 mM Tris-acetate (pH 8.1), 500 mM potassium acetate, and 150 mM magnesium acetate at 95° C. for 35 minutes. The fragmented RNA was then hybridized to a pre-equilibrated Affymetrix chip (u133 2.0) at 45° C. for 14-16 hours.

After the hybridization buffer was removed, the chips were washed in a fluidic station with a low-stringency buffer (6×SSPE [5.25% NaCl, 0.83% sodium phosphate, and 0.22% EDTA], 0.01% Tween-20, and 0.005% antifoam) for 10 cycles (two automated mixes per cycle) and in a stringent buffer (100 mM morpholinoethanesulfonic acid, 0.1 M NaCl, and 0.01% Tween-20) for four cycles (15 automated mixes per cycle), and stained with streptoavidin-conjugated phycoerythrin to identify hybridized biotin-labeled cRNA. This procedure was followed by incubation with biotinylated mouse anti-avidin antibody and re-staining with streptoavidin-conjugated phycoerythrin to amplify the signal for hybridized biotin-labeled cRNA. The chips were scanned in a HP ChipScanner (Affymetrix Inc, Santa Clara, Calif.) to detect hybridization signals. Hybridization data were normalized to an average target intensity of 500 per chip, and then analysis of induced versus un-induced PITT1 cells at baseline were performed with the program GCOS version 1.0.

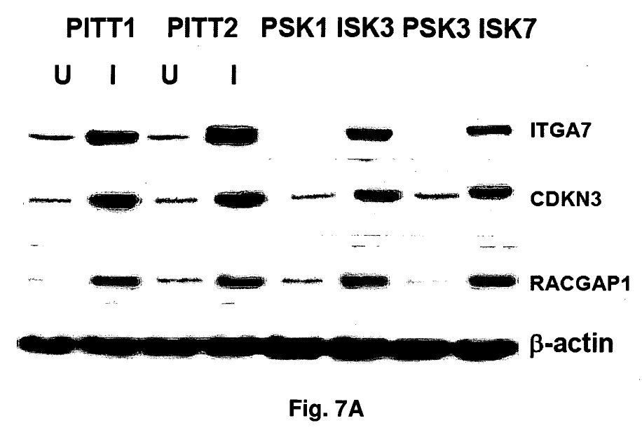

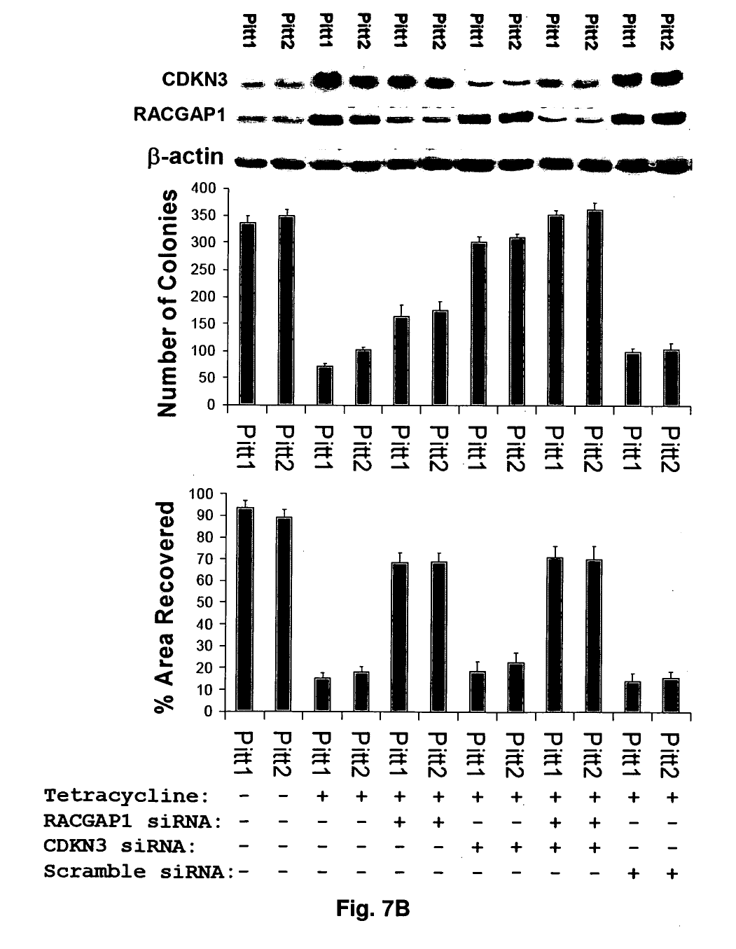

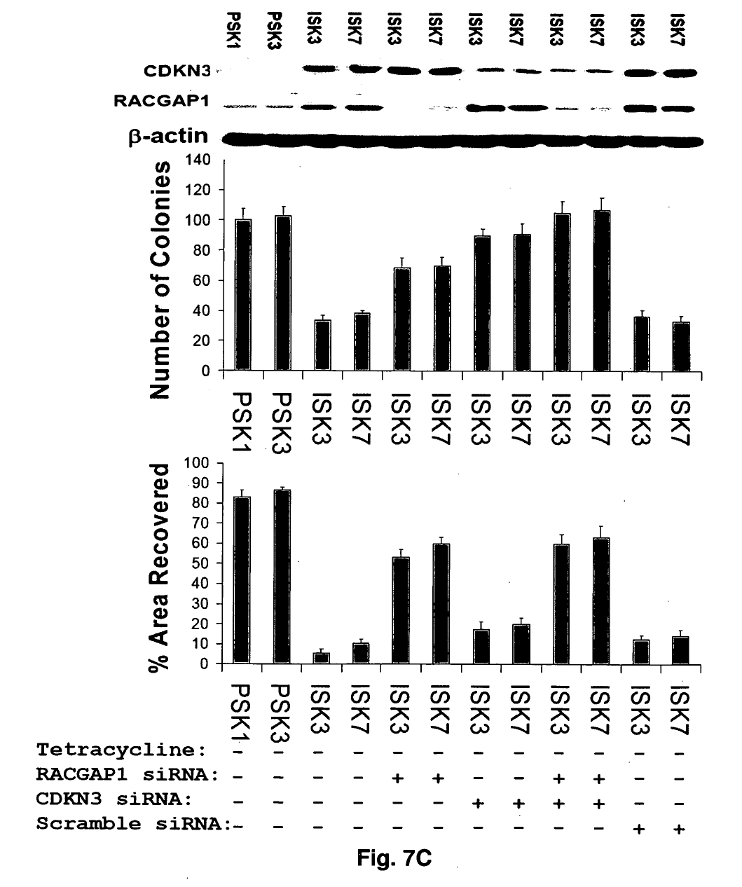

FIG. 7A shows the immunoblot analysis of integrin α7, CDKN3, RACGAP1, and β-actin expression. Lysates of pcDNA4-ITGA7-transfected PC-3 cells (PITT1 and PITT2 clones) with or without tetracycline treatment to induce the expression of integrin α7 and lysates of pCMV-ITGA7-transfected SK-UT-1 cells (ISK3 and ISK7 cells), which constitutively express integrin α7, and their corresponding vector controls (PSK1 and PSK3 cells) were electrophoresed. Proteins were transferred to a membrane and probed with antibodies specific for integrin α7 (ITGA7, rabbit polyclonal), CDKN3 (mouse monoclonal), RACGAP1 (goat polyclonal), and β-actin (as the loading control). The change in expression of integrin α7, CDKN3, RACGAP1, and β-actin was quantified based on the immunoblot analysis.

Within 24 hours of integrin α7 induction, we found a 6.1-fold (95% CI=5.5-fold to 6.8-fold) increase in the expression of CDKN3 mRNA in induced PC-3 cells transfected with pcDNA4-ITGA7 compared with un-induced cells (that is, 8593 arbitrary units in induced cells and 1408 arbitrary units in un-induced cells) and a 5.8-fold (95% CI=5.23-fold to 6.41-fold) increase in the expression of CDKN3 protein (with CDKN3/β-actin ratio increasing from 0.043 in non-induced cells to 0.249 in induced cells). We found a 5-fold (95% CI=4.26-fold to 5.70-fold) increase in CDKN3 protein expression in SK-UT-1 cells transfected with integrin α7 compared with the same cells transfected with vector control (with the CDKN3/β-actin ratio increasing from 0.042 in un-induced cells to 0.214 in induced cells). Within 24 hours of integrin α7 induction, we also found a 3-fold (95% CI=3.35-fold to 3.65-fold) increase of RACGAP1 mRNA in induced PC-3 cells transfected with pcDNA4-ITGA7 compared with un-induced cells (from 715 units in un-induced cells to 2146 units in induced cells).

Within 24 hours of integrin α7 induction, RACGAP1 protein expression was increased 2.8-fold (95% CI=2.60-fold to 3.00-fold) in pcDNA4-ITGA7-transfected PC-3 cells, compared with un-induced cells (with RACGAP1/β-actin ratio increasing from 0.049 in un-induced cells to 0.139 in induced cells]), and 3.3-fold (95% CI=2.73-fold to 3.86-fold) in SK-UT-1 cells (with RACGAP1/β-actin ratio increasing from 0.044 in un-induced cells to 0.148 in induced cells). Thus, integrin α7 expression may lead to the activation of several genes, including CDKN3 and RACGAP1.

To evaluate the importance of CDKN3 and RACGAP1 in integrin α7-mediated tumor suppressor and motility inhibition activities, we used RNA interference for CDKN3 and RACGAP1, PC-3 cells that were transfected with a tetracycline-inducible integrin α7 expression vector (pcDNA4-ITGA7), and SK-UT-1 cells that were transfected with pCMV-ITGA7 or pCMVscript. We evaluated tumor suppressor activity with the colony formation assay and motility with a wound healing assay. FIG. 7B shows the effect of RNA interference for PITT1 and PITT2 clones, where FIG. 7C shows the effect of RNA interference for ISK3 and ISK7 cell lines.

FIG. 7B shows that inhibition of CDKN3 expression by 80% in PC3 cells transfected with pcDNA4-ITGA7 and induced with tetracycline reduced integrin α7-mediated soft agar colony growth inhibition by 85% (95% CI=83.9% to 87.6%), and inhibition of RACGAP1 reduced it by 32% (95% CI=26.4% to 37.5%). When the expression of both CDKN3 and RACGAP1 was inhibited with corresponding siRNAs, integrin α7 tumor suppressor activity was virtually abolished (i.e., reduced by 99%, 95% CI=99% to 100%). Thus, the combination of CDKN3 and RACGAP1 may mediate integrin α7 tumor suppression, although CDKN3 appears to be the dominant target.

As shown in FIG. 7C, similar results were also found with the leiomyosarcoma cell line SK-UT-1. In contrast, inhibition of RACGAP1 with a RACGAP1 siRNA reversed the inhibition of motility by integrin α7 by 70%, whereas CDKN3 alone was virtually ineffective in motility inhibition (5%). The combination of CDKN3 and RACGAP1 siRNAs in PITT1 and PITT2 cells did not result in additional reversal of inhibition of motility, indicating that RACGAP1 is the main target for motility inhibition induced by integrin α7.

### DISCUSSION

To our knowledge, this is also the first report that integrin α7 appears to function as a tumor suppressor in human malignancies. Several lines of evidences support a tumor suppressor role of integrin α7 in mammalian cells.

First, in three different tumor-cell culture systems, a normal level of integrin α7 expression suppressed tumor growth, and lower levels of integrin α7 expression promoted tumor growth. In addition, mice bearing xenograft tumors from either of two highly aggressive prostate cancer cell lines had reduced tumor volume, fewer metastases, and fewer deaths if the expression of integrin α7 in the cells from which the tumors were derived was increased by transfection with integrin α7 constructs, compared with those in mice bearing xenografts from cell lines transfected with control vector.

Second, decreased integrin α7 expression was detected in human prostate tumor tissue samples and in highly aggressive soft tissue leiomyosarcoma samples by two comprehensive protein expression analyses that used data from immunostaining assays. These findings were further supported by findings from several independent microarray data sets in which prostate cancer and soft tissue leiomyosarcoma specimens expressed lower levels of integrin α7 mRNA than corresponding normal tissue specimens.

Third, integrin α7 expression appeared to activate the expression of CDKN3 and RACGAP1. CDKN3 has been shown to dephosphorylate tyrosine residues of several CDKs (including CDK2, CDK3, and CDC2) and inhibit cell cycle progression in yeast and mammalian cells (Gyuris J, et al. Cdi1, a human G1 and S phase protein phosphatase that associates with Cdk2. Cell 1993; 75(4):791-803; Hannon G J, et al. KAP: a dual specificity phosphatase that interacts with cyclin-dependent kinases. Proc Natl Acad Sci USA 1994; 91(5):1731-5). RACGAP1 has been shown to suppress growth and induce differentiation in hematopoietic cells (Kawashima T, et al. MgcRacGAP is involved in the control of growth and differentiation of hematopoietic cells. Blood 2000; 96(6):2116-24).

Thus, by activating CDKN3 and RACGAP1, integrin α7 appears to prevent cell cycle progression and suppressed tumor growth. Consistent with these findings, the expression of integrin α7 was strongest in the terminally differentiated prostate acinar cells of the prostate gland but was weakest in basal or stem cell layers of both organs, indicating that integrin α7 may prevent the overgrowth of highly differentiated tissues. This cell growth inhibition activity of integrin α7 may be mediated by activating the expression of CDKN3 and RACGAP1. Our analyses also indicated that integrin α7 inhibits cell motility and reduces metastases. Inhibition of both growth and motility may mean that integrin α7 is in a position to counteract proliferation and invasion of malignant cells.

Limitations involving the interpretation of data from cells with forced expression of integrin α7 include the artificial cultural system, variations in clonal selection, and the lack of an antitumor immune system in the mice used in our experiments. However, when integrin α7 expression in non-mutant cell lines was reduced by use of siRNA against integrin α7, the tumorigenecity of these cell lines increased, which is consistent with our hypothesis that removal of integrin α7 enhances tumorigenesis. Another limitation is that the signaling pathway used by integrin α7 to activate the transcription of CDKN3 and RACGAP1 mRNAs has not been identified. Microarray analysis indicates that PC-3 cells express other integrin α and β types in addition to integrin α7 and integrin β1, but induction of integrin α7 expression did not appear to alter the expression of other integrin molecules. Consequently, to form a heterodimer with β1 subunit, integrin α7 may have to displace mutated integrin α7 (or another integrin α) subunit from the complex, which could alter the homeostasis of integrin signaling and thus alter cell growth.

The function of integrin α7 in prostate gland and smooth muscle appears to be related to the adhesion of cells to the basement membrane and prevention of the random migration of these cells to other organs. Another important function of integrin α7 appears to be its role in limiting cell proliferation, because expression of integrin α7 induced the expression of proteins that inhibit cell cycle progression and cell growth. When the level of integrin α7 protein was decreased or the protein was mutated, cells appeared to lose inhibitory signals for both cell migration and proliferation. This loss may lead to unchecked tumor cell proliferation and a higher incidence of metastases. Thus, impairing the function of integrin α7 may be an efficient mechanism of carcinogenesis.

We claim:

1. A method of determining the presence of cancer cells in a biopsy obtained from a human, comprising determining if human integrin alpha 7 expression is reduced in cells of the biopsy is decreased, as compared to a normal control, to a level indicative of a cancer, wherein a decrease in human integrin alpha 7 function in the biopsy is indicative of cancer cells in the biopsy.

2. The method of claim 1, wherein the decrease in integrin alpha 7 expression in the biopsy indicative of cancer cells in the biopsy is a reduction in integrin alpha 7 mRNA levels to 50% or less of levels present in a normal control.

3. The method of claim 1, wherein determining if there is a decrease in integrin alpha 7 function in the biopsy indicative of cancer cells in the biopsy is performed by determining the presence of a mutation in integrin alpha 7 in a nucleic acid sample prepared from the biopsy.

4. The method of claim 3, in which the mutation is a coding mutation.

5. The method of claim 4, in which the coding mutation is a truncation or frameshift mutation of the coding sequence of integrin alpha 7.

6. The method of claim 5, in which the truncation or frameshift mutation is one of a stop codon or a frameshift mutation in codons 1-1060 of an alpha integrin 7 open reading frame.

7. The method of claim 6 in which the mutation is a stop codon.

8. The method of claim 7, wherein the mutation is chosen from one of W1060stop, W1039Stop, Q980Stop, Q921Stop, Q759Stop, Q635Stop, R569Stop, Y526Stop, Q453Stop, E350Stop, and W334Stop of SEQ ID NO: 6.

9. The method of claim 8, in which the mutation is Q921Stop.

10. The method of claim 6, in which the mutation is a frameshift mutation.

11. The method of claim 10, wherein the frameshift mutation is or immediately adjacent to codon chosen from one of codons 771, 759, 523, 502, 393, 351-353, 286 and 11 of SEQ ID NO: 86.

12. The method of claim 4, in which the coding mutation is one or more of a missense mutation, point mutation, nonsense mutation, deletion mutation, or insertion mutation of an integrin alpha 7.

13. The method of claim 4, in which the coding mutation occurs in exon 21 of the integrin alpha 7 region.

14. The method of claim 4, wherein the mutation is an insertion mutation in exon 11 of the integrin alpha 7 region.

15. The method of claim 3, wherein the mutation is chosen from MIK, G725R, and a deletion of V137 of SEQ ID NO: 87.

16. The method of claim 3, wherein a nucleic acid amplification assay is used to determine the presence of a mutation in integrin alpha 7 in the biopsy.

17. The method of claim 16, wherein the nucleic acid amplification assay comprises one of a PCR, a reverse transcriptase PCR (RT-PCR), an isothermic amplification, a fluorescent energy resonance transfer (FRET)-based assay, a nucleic acid sequence based amplification (NASBA), a 5′ fluorescence nuclease assay, a molecular beacon assay, a microarray assay, and a rolling circle amplification assay.

18. The method of claim 1, wherein the cancer is one of prostate cancer, glioblastoma multiforme, leiomyosarcoma, or hepatocellular carcinoma.

19. The method of claim 1, wherein determining the presence of cancer cells in the biopsy is used for diagnosing a metastasis or a potential for cancer relapse in the patient.

20. The method of claim 1, wherein determining if there is a decrease in integrin alpha 7 expression in the biopsy indicative of cancer cells in the biopsy is performed by an immunohistochemical assay.

21. The method of claim 1, further comprising determining if cyclin kinase inhibitor 3 expression is decreased at least 50% in the biopsy as compared to a control.

22. The method of claim 16, further comprising determining if rac GTPase-activating protein 1 expression is decreased at least 50% in the biopsy as compared to a control.

23. A kit comprising packaging containing a container containing a primer adapted to amplify or sequence a portion of an open reading frame of human integrin alpha 7 containing one or more of codons 1, 11, 137, 286, 334, 350, 352, 393, 453, 502, 523, 526, 569, 635, 759, 771, 921, 980, 1036, and 1060 of SEQ ID NO: 86, and at least 5 nucleotides flanking those codons.


SEQUENCE LISTING (90 sequences)

SEQ ID NO: 1 - protein, 19 aa, Artificial Sequence
  Synthetic peptide corresponding to amino acids 1097-1115 of human integrin alpha 7
GTILRNNWGSPRREGPDAH

SEQ ID NO: 2 - DNA, 20 bp, Artificial Sequence
  Purified genomic DNA or cDNA corresponding to exon of ITGa7
tgcggctgctgtagttgtcc

SEQ ID NO: 3 - DNA, 21 bp, Artificial Sequence
  Purified genomic DNA or cDNA corresponding to exon of ITGa7
aaggtagcaaatcccggaggc

SEQ ID NO: 4 - DNA, 21 bp, Artificial Sequence
  Purified genomic DNA or cDNA corresponding to exon of ITGa7
gcctccgggatttgctacctt

SEQ ID NO: 5 - DNA, 21 bp, Artificial Sequence
  Purified genomic DNA or cDNA corresponding to exon of ITGa7
atgaggaggcccacagagtgg

SEQ ID NO: 6 - DNA, 22 bp, Artificial Sequence
  Purified genomic DNA or cDNA corresponding to exon of ITGa7
tgacctctaactcctgtccctg

SEQ ID NO: 7 - DNA, 21 bp, Artificial Sequence
  Purified genomic DNA or cDNA corresponding to exon of ITGa7
tctgttcatgcagggccacac

SEQ ID NO: 8 - DNA, 21 bp, Artificial Sequence
  Purified genomic DNA or cDNA corresponding to exon of ITGa7
cctaattcccagtgtcctgcc

SEQ ID NO: 9 - DNA, 21 bp, Artificial Sequence
  Purified genomic DNA or cDNA corresponding to exon of ITGa7
ccccatccgtgcattcagtca

SEQ ID NO: 10 - DNA, 21 bp, Artificial Sequence
  Purified genomic DNA or cDNA corresponding to exon of ITGa7
cctggcccacagagtgaaatg

SEQ ID NO: 11 - DNA, 21 bp, Artificial Sequence
  Purified genomic DNA or cDNA corresponding to exon of ITGa7
tccccaccatccaactcatcc

SEQ ID NO: 12 - DNA, 21 bp, Artificial Sequence
  Purified genomic DNA or cDNA corresponding to exon of ITGa7
ggatgagttggatggtgggga

SEQ ID NO: 13 - DNA, 21 bp, Artificial Sequence
  Purified genomic DNA or cDNA corresponding to exon of ITGa7
gaggttttggtccccttctcc

SEQ ID NO: 14 - DNA, 21 bp, Artificial Sequence
  Purified genomic DNA or cDNA corresponding to exon of ITGa7
tactctggatgtcccctccct

SEQ ID NO: 15 - DNA, 21 bp, Artificial Sequence
  Purified genomic DNA or cDNA corresponding to exon of ITGa7
tccaggaggtgggagcttaca

SEQ ID NO: 16 - DNA, 21 bp, Artificial Sequence
  Purified genomic DNA or cDNA corresponding to exon of ITGa7
taggggtaagtcacccttccc

SEQ ID NO: 17 - DNA, 21 bp, Artificial Sequence
  Purified genomic DNA or cDNA corresponding to exon of ITGa7
cctctacccactcacccatca

SEQ ID NO: 18 - DNA, 21 bp, Artificial Sequence
  Purified genomic DNA or cDNA corresponding to exon of ITGa7
gggaggacccacactgaatgt

SEQ ID NO: 19 - DNA, 21 bp, Artificial Sequence
  Purified genomic DNA or cDNA corresponding to exon of ITGa7
ctttccagttccccgtcacac

SEQ ID NO: 20 - DNA, 21 bp, Artificial Sequence
  Purified genomic DNA or cDNA corresponding to exon of ITGa7
gtgactgccttttccctgtgc

SEQ ID NO: 21 - DNA, 21 bp, Artificial Sequence
  Purified genomic DNA or cDNA corresponding to exon of ITGa7
gattccacccacacccattcc

SEQ ID NO: 22 - DNA, 21 bp, Artificial Sequence
  Purified genomic DNA or cDNA corresponding to exon of ITGa7
gaggctgacagctggttctct

SEQ ID NO: 23 - DNA, 21 bp, Artificial Sequence
  Purified genomic DNA or cDNA corresponding to exon of ITGa7
ggaaaaggttgagaggggctc

SEQ ID NO: 24 - DNA, 21 bp, Artificial Sequence
  Purified genomic DNA or cDNA corresponding to exon of ITGa7
gtgctcttgactccccaatcc

SEQ ID NO: 25 - DNA, 21 bp, Artificial Sequence
  Purified genomic DNA or cDNA corresponding to exon of ITGa7
aaggatcaaagggagggcagg

SEQ ID NO: 26 - DNA, 21 bp, Artificial Sequence
  Purified genomic DNA or cDNA corresponding to exon of ITGa7
ttggctcaggagccacctttg

SEQ ID NO: 27 - DNA, 21 bp, Artificial Sequence
  Purified genomic DNA or cDNA corresponding to exon of ITGa7
aaacccaaaagggcgagccac

SEQ ID NO: 28 - DNA, 21 bp, Artificial Sequence
  Purified genomic DNA or cDNA corresponding to exon of ITGa7
cctcctttcccaacatgccac

SEQ ID NO: 29 - DNA, 21 bp, Artificial Sequence
  Purified genomic DNA or cDNA corresponding to exon of ITGa7
aagccaaggggtcagtgtcca

SEQ ID NO: 30 - DNA, 21 bp, Artificial Sequence
  Purified genomic DNA or cDNA corresponding to exon of ITGa7
ctggggattgttccagtgagg

SEQ ID NO: 31 - DNA, 21 bp, Artificial Sequence
  Purified genomic DNA or cDNA corresponding to exon of ITGa7
gggctaaaccagaacccatgc

SEQ ID NO: 32 - DNA, 22 bp, Artificial Sequence
  Purified genomic DNA or cDNA corresponding to exon of ITGa7
ccctaggaatgccccttatctc

SEQ ID NO: 33 - DNA, 21 bp, Artificial Sequence
  Purified genomic DNA or cDNA corresponding to exon of ITGa7
cttgaactcttgccctcccac

SEQ ID NO: 34 - DNA, 21 bp, Artificial Sequence
  Purified genomic DNA or cDNA corresponding to exon of ITGa7
tagcaggagtggggtctgact

SEQ ID NO: 35 - DNA, 20 bp, Artificial Sequence
  Purified genomic DNA or cDNA corresponding to exon of ITGa7
tcaagaccccaccccatcct

SEQ ID NO: 36 - DNA, 21 bp, Artificial Sequence
  Purified genomic DNA or cDNA corresponding to exon of ITGa7
ccttgccttctctcccattcc

SEQ ID NO: 37 - DNA, 21 bp, Artificial Sequence
  Purified genomic DNA or cDNA corresponding to exon of ITGa7
agggataagggcagatgtgcc

SEQ ID NO: 38 - DNA, 19 bp, Artificial Sequence
  Purified genomic DNA or cDNA corresponding to exon of ITGa7
tagaccacccctgactcta

SEQ ID NO: 39 - DNA, 21 bp, Artificial Sequence
  Purified genomic DNA or cDNA corresponding to exon of ITGa7
tatgactacccccacctcacc

SEQ ID NO: 40 - DNA, 21 bp, Artificial Sequence
  Purified genomic DNA or cDNA corresponding to exon of ITGa7
atacttgcccctgcccactca

SEQ ID NO: 41 - DNA, 21 bp, Artificial Sequence
  Purified genomic DNA or cDNA corresponding to exon of ITGa7
ggaaatgtcaatgccccctcc

SEQ ID NO: 42 - DNA, 21 bp, Artificial Sequence
  Purified genomic DNA or cDNA corresponding to exon of ITGa7
gaccttctcacccctgttctg

SEQ ID NO: 43 - DNA, 20 bp, Artificial Sequence
  Purified genomic DNA or cDNA corresponding to exon of ITGa7
gggcctcatccctgacactt

SEQ ID NO: 44 - DNA, 21 bp, Artificial Sequence
  Purified genomic DNA or cDNA corresponding to exon of ITGa7
ggtctctcccctcatactctc

SEQ ID NO: 45 - DNA, 21 bp, Artificial Sequence
  Purified genomic DNA or cDNA corresponding to exon of ITGa7
tgtccccacatctaaccccca

SEQ ID NO: 46 - DNA, 21 bp, Artificial Sequence
  Purified genomic DNA or cDNA corresponding to exon of ITGa7
gctgtgattggagggacactc

SEQ ID NO: 47 - DNA, 20 bp, Artificial Sequence
  Purified genomic DNA or cDNA corresponding to exon of ITGa7
tctggctgcaccgagtctgg

SEQ ID NO: 48 - DNA, 21 bp, Artificial Sequence
  Purified genomic DNA or cDNA corresponding to exon of ITGa7
agtggcttagacccctgtctg

SEQ ID NO: 49 - DNA, 21 bp, Artificial Sequence
  Purified genomic DNA or cDNA corresponding to exon of ITGa7
ctagagccgagtggtatcctc

SEQ ID NO: 50 - DNA, 21 bp, Artificial Sequence
  Purified genomic DNA or cDNA corresponding to exon of ITGa7
aagggtctccttccctgttcc

SEQ ID NO: 51 - DNA, 20 bp, Artificial Sequence
  Purified genomic DNA or cDNA corresponding to exon of ITGa7
acctatcccccaaccctgca

SEQ ID NO: 52 - DNA, 21 bp, Artificial Sequence
  Purified genomic DNA or cDNA corresponding to exon of ITGa7
tgctccattgaccccttgctc

SEQ ID NO: 53 - DNA, 21 bp, Artificial Sequence
  Purified genomic DNA or cDNA corresponding to exon of ITGa7
tgctcacccaaccaggaagtc

SEQ ID NO: 54 - DNA, 21 bp, Artificial Sequence
  Purified genomic DNA or cDNA corresponding to exon of ITGa7
gctcttcaggctcctcatggt

SEQ ID NO: 55 - DNA, 21 bp, Artificial Sequence
  Purified genomic DNA or cDNA corresponding to exon of ITGa7
tcaggatggtgcccgtcttct

SEQ ID NO: 56 - DNA, 21 bp, Artificial Sequence
  Purified genomic DNA or cDNA corresponding to exon of ITGa7
agaagacgggcaccatcctga

SEQ ID NO: 57 - DNA, 21 bp, Artificial Sequence
  Purified genomic DNA or cDNA corresponding to exon of ITGa7
tcttgatgcgacaccagcagc

SEQ ID NO: 58 - DNA, 21 bp, Artificial Sequence
  Purified genomic DNA or cDNA corresponding to exon of ITGa7
gctgctggtgtcgcatcaaga

SEQ ID NO: 59 - DNA, 21 bp, Artificial Sequence
  Purified genomic DNA or cDNA corresponding to exon of ITGa7
cttggggtcctgttacacagg

SEQ ID NO: 60 - DNA, 21 bp, Artificial Sequence
  Purified genomic DNA or cDNA corresponding to exon of ITGa7
cctgtgtaacaggaccccaag

SEQ ID NO: 61 - DNA, 22 bp, Artificial Sequence
  Purified genomic DNA or cDNA corresponding to exon of ITGa7
gcaagactcaaagaggcagagg

SEQ ID NO: 62 - DNA, 21 bp, Artificial Sequence
  Purified genomic DNA or cDNA corresponding to exon of ITGa7
gcactaacaggtctgtccttg

SEQ ID NO: 63 - DNA, 21 bp, Artificial Sequence
  Purified genomic DNA or cDNA corresponding to exon of ITGa7
agagggttagagcagttctgg

SEQ ID NO: 64 - DNA, 22 bp, Artificial Sequence
  Purified genomic DNA or cDNA corresponding to exon of ITGa7
gatttcccttgcattcgctggg

SEQ ID NO: 65 - DNA, 24 bp, Artificial Sequence
  Purified genomic DNA or cDNA corresponding to exon of ITGa7
tgccctgctggcagaacccaaatt

SEQ ID NO: 66 - DNA, 23 bp, Artificial Sequence
  Purified genomic DNA or cDNA corresponding to exon of ITGa7
gagggacgcccccaaggccatga

SEQ ID NO: 67 - DNA, 24 bp, Artificial Sequence
  Purified genomic DNA or cDNA corresponding to exon of ITGa7
ggaaagccatcttggttgaggtcc

SEQ ID NO: 68 - DNA, 24 bp, Artificial Sequence
  Purified genomic DNA or cDNA corresponding to exon of ITGa7
tgactccatgttcgggatcagcct

SEQ ID NO: 69 - DNA, 22 bp, Artificial Sequence
  Purified genomic DNA or cDNA corresponding to exon of ITGa7
ggacaaggtcactacaatggcc

SEQ ID NO: 70 - DNA, 22 bp, Artificial Sequence
  Purified genomic DNA or cDNA corresponding to exon of ITGa7
tgtggagacgccatgttccagc

SEQ ID NO: 71 - DNA, 23 bp, Artificial Sequence
  Purified genomic DNA or cDNA corresponding to exon of ITGa7
ctcaatgctgatcccggaggtgc

SEQ ID NO: 72 - DNA, 24 bp, Artificial Sequence
  Purified genomic DNA or cDNA corresponding to exon of ITGa7
cccaggtcaccttctacctcatcc

SEQ ID NO: 73 - DNA, 24 bp, Artificial Sequence
  Purified genomic DNA or cDNA corresponding to exon of ITGa7
ctgtagagtgggcagctgaacacc

SEQ ID NO: 74 - DNA, 24 bp, Artificial Sequence
  Purified genomic DNA or cDNA corresponding to exon of ITGa7
ggccagtgtcctctgctgagaaga

SEQ ID NO: 75 - DNA, 22 bp, Artificial Sequence
  Purified genomic DNA or cDNA corresponding to exon of ITGa7
caggctgggacatgggaaccta

SEQ ID NO: 76 - DNA, 57 bp, Artificial Sequence
  oligonucleotide corresponding to region of CDKN3 mRNA
caccggagcttacaacctgccttaaattgatatccgtttaaggcaggttgtaagctc

SEQ ID NO: 77 - DNA, 57 bp, Artificial Sequence
  oligonucleotide corresponding to region of CDKN3 mRNA
aaaagagcttacaacctgccttaaacggatatcaatttaaggcaggttgtaagctcc

SEQ ID NO: 78 - DNA, 57 bp, Artificial Sequence
  oligonucleotide corresponding to region of RACGAP1
caccgtttgcactttggatgctgaaattgatatccgtttcagcatccaaagtgcaaa

SEQ ID NO: 79 - DNA, 57 bp, Artificial Sequence
  oligonucleotide corresponding to region of RACGAP1
aaaatttgcactttggatgctgaaacggatatcaatttcagcatccaaagtgcaaac

SEQ ID NO: 80 - DNA, 59 bp, Artificial Sequence
  oligonucleotide corresponding to region of ITGA7
caccgactcccaaccactggttctccttgccgaagcaaggagaaccagtggttgggagt

SEQ ID NO: 81 - DNA, 59 bp, Artificial Sequence
  oligonucleotide corresponding to region of ITGA7
aaaaactcccaaccactggttctccttgcttcggcaaggagaaccagtggttgggagtc

SEQ ID NO: 82 - DNA, 57 bp, Artificial Sequence
  Scrambled siRNA
caccgtaatgtattggaacgcatattttgatatccgaatatgcgttccaatacatta

SEQ ID NO: 83 - DNA, 56 bp, Artificial Sequence
  Scrambled siRNA
aaaataatgtattggaacgcatattcggatatcaaaatatgcgttccaatacatta

SEQ ID NO: 84 - DNA, 63 bp, Artificial Sequence
  T7-d(T)24 primer for cDNA synthesis
ggccagtgaattgtaatacgactcactatagggaggcggtttttttttttttttttttttttt

SEQ ID NO: 85 - DNA, 24283 bp, Homo sapiens
ccagtccaggactctgccccctcccatcccctttcatggataggaaatgtgcagtcctgggacgggtctggtagctggggacaccctttacatccctctgcctcttgggtccagtctctttcatctttgccttctttgacacccactcccctccccactgcttaatttcctcttcctgtaatcatccccagtcgttttcttttctcccttcattccatcccttgtcaattaatctcttgcccttctttcttcctctctattcctttcctttttccatttctccatttgctccccgtatctcccgagtttctctctctcttcttgcctctttttctctgttcccttgaatcctgacgatgtggctagcactgctgtggtcattgccgggctgggggcgggggatgggataggatgggggagggcagcggtctgatcccaacagcagaaagagtgctctatgtgaccatgggggaacagggagcactaagatgccacgctgcacccaggcccaggacggctcccctttcatttcctctctatctgcacatctctcttcccaggttgtcttttagcgtcttcccaacttctcatctcttaccctccttcctctgtttcagcccctctctttctatctgtacttctctccctccgcattccaaggcgccgcctccaccactcccggggtggggatggggttgggggagaaggggaggagagcgccgcgcaggggcggagccggagacggtgctgggcttggggggcgtggtggtggggggtcagcaaggctagtttccatcccagccaccagcctgggcatccccttggagacgggcttgggtctccacctgccgcgggagcgaggggcggggccggaggcggggcctgagtggcgtccccgggagaggaggcgggagccggagtgggcgccggagctgcggctgctgtagttgtcctagccggtgctggggcggcggggtggcggagcggcgggcgggcgggagggctggcggggcgaacgtctgggagacgtctgaaagaccaacgagactttggagaccagagacgcgcctggggggacctggggcttggggcgtgcgagatttcccttgcattcgctgggagctcgcgcagggatcgtcccatggccggggctcggagccgcgacccttggggggcctccgggatttgctacctttttggctccctgctcgtcgaactgctcttctcacgggctgtcgccttcaatctggacgtgatgggtgccttgcgcaaggagggcgagccaggcagcctcttcggcttctctgtggccctgcaccggcagttgcagccccgaccccagagctggtgagtcaccgcacccgcccagagtcgccatgcccgagccacagatcgtccccctccccactctgtgggcctcctcatttctctgttttctagccccaccaagacctagactgcccacagacatcccacatcccaacctggagccttgcctcatctggcttgcgtctgaagctgcacttcccggccctgagaccagtattttgctttagggatgagttggaaagcaaggttcttgtcttggcagcgaaccatctccttcttctgggcctttcccccaacttgcatccttgatccagccccagggcctctggctcccctgcttcttccaagggctgaattccccaagggagggagactgtctgtctctgcttagaatgggaggagatggaaaggacatagaagttgagggtgccatgagagggatgcatgcagggcagactccagaaataacttcctgctagagcattgccatggatggaatgagggcagcagggcactggaaggccaggagagagcttccacttctgtggcttaagaccacgggaagattgggagaggatctgcaggtctgccaacctgcagtaggtggcttggtgatagagagtggcagcaaactgaaccctcaaagtactagtagcagtagtagtagccgcagcagctgtagcagtgagagagatccaggaaggatgctggccaggctgctccccttcctcctccttagcaaatttccaactccaggaatctcagcagctgggaagggccaggaggagtaaggggtggaggacaattctaattttttctaatcagttcaggacccatgggagatggatatacttttgtgaggggcctgtgactggtcatgttgcctgtatccttggctcttgctacatgtctgattgtaaaaagggaggccagaggtgaagaaagcttctcacctgctcctgctagggggcttttctctcttcaaccagtgcctaagccacattaagtatccattactgggatcaatgctgtccactgggactgtcttctgcctctactgtcggtctgggggcagggggcagggacaagagctcatttctcctcacttgcttggggagtgggggcctagctctaatctttcttcttccattatccctatcatctggtagcagggtcggtggtccccaaaactttgggagagatagaaagcaacggacttcatctcctcttctgtttaccatctgcttcctcattcacctttgctccctccctcccttcctccctccttctccatctgtcagagttcgaggactggaggcctttttaggacatgctgaactctctaagctatttccaggcaaattctaggttatttttaatagcttggtctcttgtcatttccccctcctctctgaaggtggcccctggttccgtctcccagagccaagctggggcctttcccagagggcctgactgcctcaccctgcttttgctccagcagggggtgctctgctgctggggggcggggggtatgtgagaggccaggcacctgctcagtccctagcttttgagttgcaggtggcctgccttagcactcactgatgaaaaaaacttcttgcctgttttgatgtcttttagtctagctctgggatgagactttaaggtctaacctttgctgtgtggttccagcctcatttacttccctcaactgtaaaaaggatataaacatagtattactacatagggttgttgtgcagattaagagttcttaatatatataaaatgcttagaatagtgcatagcccgcagtgagtgctgtgaagtgttagtagtattgctattcttgtattgtgattcacagagcgccttacagagattctggatccaaaggcttggctagagggcctccctggctgagccagccttccaggccaagcatcctccccagagggccacccagattgagaggggccaaagaggggctggacttgggctggggccctggagtgtgtggagaatcgagaagtgcagtggtcgtgggctactcctttgtcttcacttagctgagctcccagggggtccctctgccccccagctgccaacacttttttttttttttttttgcttttctctctgcagtggctacactgtggctgtccagaagactggggtggttagggcgtatggcatgaagccaggaaggagtgtgtgtggctggaccagaggtggagggactagagaggatgctgctgggtgctcttgttccactaaggatcgattggtctcttctccaccaagagcggactgggcatatctatgcactcagcttctttcttccacatgggcccctcccctcctccctacttttggcctccagaggagatgtgaacatagaacaaggataacttatctgggtgcttagctatgcactgaccagctgtgacactgggtatctctatgagtccacaaaatgtgtgtgttcagtaaacacattctgacactccctatggggcaagcacaaagatgaaaagacagccccgacactcagagagatggccacttctatgtttggaggctgggggtactgctgacttgcctgaaggttgccatttatttatgcagggctgtatcaccccgtttccttttctgcccagggtaccctcatctccccactctctccttccctttctggggtggtctcagtgttctagagacaggtcagtcactgggtggagtgacaaagtgttggagttaggcccatgtggatttgaattccagcatcactgcttaatgtctttgagtgagttttctcatctgaaagacaagaaaagaatccttatctcataggattgttctgatgattaaatgacataatgcatgtgacttgcctatcctggtgcttggcacatatgtggacagtgatgaatgttagtttcttatatccctggtgtctagcctcggatctgacgtcatagtaggtgctgaataaatatgatttccttgtctcaccagcgcctgacacagggcttggcatacaatagactctcaataagtagttgaatgccaaatgtgtcttctcttctctactactccctataccccttctctgtcttgacactggctctgacaagggatggcagctgctaagagatgaggaggagttgtgggaaggaagaatggctctctgccctccccctccaccccatcagagctggcacagtgccccacagatgcctgtctgtaatactgcctaacatggttttgggccttgccccccaggaagggagatggaggagaagagtgtgggagagaggcgttgaggtttgtcccactgccacttctgagtctctccttctgcaaagagaggacccatggagccagctgggtgtcagtcatcttacctcacccccgccttcactctggcttgggggttcagccccaggggacccaggcagcctccattcccagcactgtgctcccctggggaagacggcttggctgtgatcatggaaaattgtcctgccaagaaagttgtagctgggaaagaggctgagggggaggcaggagagaagactgggtgggggtggaagggaaggagaaatcatggacatggggagaaggaaggatggggaaggggattcaggatgtcgggaagagaatggggtagcattggaggcagaaggagaaacttgtccctacctccatgccagccagagtgactgatggaatcctgggctggcacagcttctgggaggtggggtctttgctgggtccctgatgagggggcagtgggtccgtatctagcctcttgcctggcctctgaagctggtccctgagccacactctgatgccagtctggggccctgttacttttgctcccagcattcttggcatttctggctgggtttcaactggactgggttggggagcagggcagagcttggggatggggccaaggaggggatagggaaggcctactcaggaacaggtgctgggaacaggcagttctttcaaaccagcactgttggcctggctgcttgggttggcgtgtatgtgtgtgtgtgtgtgtgtgtgtgtatctactgtgtatgttgatcccttatccagatagtatgtacatgcaacgtgatgactgcatgaccaagcatattaatttgtccttgccagggtttgagaaaactgacatttgccccttctctttagtccttgaacactctctttagtactgaggggttgggcctgggcagctctaatgagattgggtcattctgacctctaactcctgtccctgtccctgcccctgccccatcttgcaggctgctggtgggtgctccccaggccctggctcttcctgggcagcaggcgaatcgcactggaggcctcttcgcttgcccgttgagcctggaggagactgactgctacagagtggacatcgaccagggaggtgtggccctgcatgaacagagtgggggaagcgtgtgagcggggaggagaggacttgggctcctcttccctcccctaattcccagtgtcctgcctctagctgatatgcaaaaggaaagcaaggagaaccagtggttgggagtcagtgttcggagccaggggcctgggggcaagattgttgtgagtattgcttctcatgactgaatgcacggatggggtgtgtgtgtgtgtgtgtttatggtgtgtgcatacgcataggtgtgcttagagaacacaagttaggaatatggtatgattccaagtacatcagggagatataaaaaggtgtgagacatggtccttgtccttataaatgtaaaaatgtctgtccattcattcatccatccatttgtcaaactcttactgagaaccttttaagcatcaggcattgtgctagttactacaggggaaggctcatgcctgtaatcccagcactttcggaggccgaggcaggtggatcacctgaggtcaggagttcgagaccagccggaccaacatggcgaaaccctgtctctcctaaaaatacaaaaaaattagccgggcgtggccgggtgcggtggctcacgcttgtaatcccagcactttgggaggccgaggtgggtggatcacgaggtaaggagatcgagaccatggtgaaaccccgtctctactaaaaacacaaaaaattagccgggcgtggtggcgggcgcctgtagtcccagctactcagagaggctgaggcaggagaatggcgtgaaccggagaggcggagcttgcagtgagctgagatcgcgccactgcgctccagcctgggtgacagagcgagactccgtctcaaaaaaaaaaaaaaattagctaggtgtggtggcaggcgcctgtaatcccaggtactcgggaggctgaggtaggagaatcacttgaacctgggtggaggaggttgcagtgagctgagatcgcaccattgtaccctagcctgggagacaagagcaaagttccgtctcaaaaccaaccaaacaaacaaacgaaaaaaccagagctctctgtttctctctctctctctatctttcagtaacacgcatagatacacaattaccaatacagatcactgtggggcagaatctggttcatgttaagtgagtggtctagtctccagtctataaaagtccaaaggaggagtagagagaagacttctgcagaggggatgatttgagccaggctttaataataggtaatacctagcctgtgcaacatagtgggacctcatctttataaaaaataaaaacaaattagccagtcatggtggtgcatgcctgtagtcccagctacacaggaggctgaggtgggaggatcacttgagccctggaggtcgaggctgcagtgagccatgattgtgccactgcactccagcctgggtgacagagtgagaccttgcttcaaaaaaaaaaaaaaagtaatacttggagagtgaagcggacaggaagttctttgcagatgagatggtgacacttacaaaggtccagggacagggccaagcttggcattttggaggactgtgacatgatcagggagacacacatcctatgtggtggcttaattgtgtcttttggctccaggcagaatgtggaacaaggagatctccatttgagggcaaggaagtgggtgcagacaggttgctgggttatgcatggacctgtgtaacactggcagggtaatggtgcttgagtggtgccggcataggggtgtgtgtgtatgtgtgcatgtgcatgtgcatgtgagcacacatgtatcagtatctgccaaatctctgcatatgggcagcatgcctcaagcaggtccctggcccacagagtgaaatgatccccatcccttcctcccccagacctgtgcacaccgatatgaggcaaggcagcgagtggaccagatcctggagacgcgggatatgattggtcgctgctttgtgctcagccaggacctggccatccgggatgagttggatggtggggaatggaagttctgtgagggacgcccccaaggccatgaacaatttgggttctgccagcagggcacagctgccgccttctcccctgatagccactacctcctctttggggccccaggaacctataattggaagggtgagtcactcctcgggaaggggagaaggggaccaaaacctcctcttacctcagagacagggttggggatggcacatggccaagcatgaccacatgtgcactgctgtatggccccagggcactgccatgccttccaccccattgagctagtgcacacatgaatggggggtgcctcctttccctcgcacggccaagtgttcctcaacatgctggcatgggccccaagtgcacgctgggcctgcagctggggcctgcatgctccaacacactagcccacacctcatcactgccattcccgtctccgcacgctgctgctggctgagctgacactcggtgagtgtgatgccacatctgggggaccccaggaagcctgggttggggacagggtggggagagggctagaaagaagaggcagggcttccccgtgtgcctgtctaactcagtgtccggcctgaggggtgttccttgcgccctgccctgggcactaacaggtctgtccttgcaggcacggccagggtggagctctgtgcacagggctcagcggacctggcacacctggacgacggtccctacgaggcggggggagagaaggagcaggacccccgcctcatcccggtccctgccaacagctactttggtagggacctctccccggcccagaactgctctaaccctctgctcctctctcttgtcctctctctccatgctcccatccttctgtctctgtttctgtctctcaccttgtctctctctgtctttctgtctctggctgtgatctctctggtctctttttctctctccacctcttcttcttccaccattttctggcctttctgtggctctgtctccctactctgtggcccctactctggatgtcccctccctggtgtctcaccccaccccccacagggttgctttttgtgaccaacattgatagctcagaccccgaccagctggtgtataaaactttggaccctgctgaccggctcccaggaccagccggagacttggccctcaatagctacttaggtttgtaagctcccacctcctggactctaggggcatggcccagcctcccctccttccccagggaactcgacctttggtgccttataatctcctcctcccccaacacacacccagggagacatacattgggcccaaattgcagagaagagctgggtccaatgatcaggcctaagaggaggaggcccccagggtggtggcctctggggctgtgagccaggggtctccatggaggaagattcaggtggaatgagagggccagggctgaggatattttgggaaggacagtcctgtcttctagggggactttccctgaggggatggatggtgggcacatattgaagaaagggctaatgttgttggtaagtccctctcgttgtctcatctgcattcctctgcagaggaggaggaaaccaggcctgggagatgtttgggtgaagcaggcgctctctcactcccccttgtctccccctcatccatgtgaagacttcccctccctgccaggatgagggagttgggggaaagaggtgcactgggtgggattcgggcctgagagggacctctagctcttctagctccctgggtgtgggcagggtgaggccactgtgctcagcctcctacctgggctcctggccttctcagccatcacctttctctctcttgcccagtccctgaggctgacctcactgcactttttgtgccaagcttgtctctgggcctggtgggtgtgggaggctgccaggccctgtggggaggaagagctatccagctgtggtgctgatgacttggggggacctatcttttggctcttaacctaggggagggggcagggtgcaggggagctgtgacttggctcttaacctgtagggagggggcaggggctgggggagctgtgacacaccccagcttctgagtcttggggtgaagacttaggggtaagtcacccttcccccaggcttctctattgactcggggaaaggtctggtgcgtgcagaagagctgagctttgtggctggagccccccgcgccaaccacaagggtgctgtggtcatcctgcgcaaggacagcgccagtcgcctggtgcccgaggttatgctgtctggggagcgcctgacctccggctttggctactcactggctgtggctgacctcaacagtgatgggtgagtgggtagagggccgtgccacctgagggaggctgggtctagtagccccagtctggctgaggccacttagcctcctgctggctcctctggccagggaggacccacactgaatgtttccctctctccatagctggccagacctgatagtgggtgccccctacttctttgagcgccaagaagagctggggggtgctgtgtatgtgtacttgaaccaggggggtcactgggctgggatctcccctctccggctctgcggctcccctgactccatgttcgggatcagcctggctgtcctgggggacctcaaccaagatggctttccaggtgtgacggggaactggaaaggctcagggagggaggggccacaggagggatggggaagcccctcagaggtcagggtgtggtcttctgaggactcagggagagagggtccctgagcttatgtctgagctgtaccatttaccagctttctgaccttggcaagttcctaacctttttgcgttagtaatatctgcagggagtggccaagaggattaaagatgatgtatgtagagtgcctgggattttgtagcctctcaataaaatagaaaacatacctgagtgactggggggagttgaggcctggatcttgtctgcaaggcccccagccagcgtgactgccttttccctgtgccctgcagatattgcagtgggtgccccctttgatggtgatgggaaagtcttcatctaccatgggagcagcctgggggttgtcgccaaaccttcacaggtgaggggagtcgctgggatgagggaatgggtgtgggtggaatcagcagaggcatcaggggaggcagaggcctgcgggaggtgggattgagggaggctgacagctggttctctaggtgctggagggcgaggctgtgggcatcaagagcttcggctactccctgtcaggcagcttggatatggatgggaaccaataccctgacctgctggtgggctccctggctgacaccgcagtgctcttcaggtgagcccctctcaaccttttccctccctgaggccgtcagcccctccctgtgactctgaccccgacctcagtgccaaatctaatgctgaagagtgtttcccagcctcatgttctcatgtttcttgtgctcttgactccccaatcccagggccagacccatcctccatgtctcccatgaggtctctattgctccacgaagcatcgacctggagcagcccaactgtgctggcggccactcggtctggtgaggtgggatcgggtggcacctggaccctggcagcttcccctgccctccctttgatcctttatctccccagtttggggctggggctgtgacaggatgtgacagatggggtgggggtaggggccttggcccatcagcctcgtttggctcaggagccacctttgcccccgcagtgtggacctaagggtctgtttcagctacattgcagtccccagcagctatagccctactgtgggtgagtgcggtcccccctctgtggctcgcccttttgggtttcccagggaggggggtcacttcgaggtggtagaagagcacccttggaatggggtgagctggagcagctctgcagctcagcagctcctcctttcccaacatgccacagccctggactatgtgttagatgcggacacagaccggaggctccggggccaggttccccgtgtgacgttcctgagccgtaacctggaagaacccaagcaccaggcctcgggcaccgtgtggctgaagcaccagcatgaccgagtctgtggagacgccatgttccagctccaggtggacactgaccccttggcttctgagggtcattttcatggctccatctcttttccctgattcctcttagctgctttttcccgagcacactcgtgcctccttctaagacctagacacgtgggaaacctgtcttctgagctcacttcctcctcatctgctgccctctcctgtcatttctgcactccctggaggaggaggaggtgcaaggggcttcatgtcccctcttccagctgaatggaggaggtggcagcttttatcatcacatctggtcctcaaagccctaaaggtttcccctgtcccctaccccagtctttctccctcctccttgctcagttctccacctctctcattcatctccctaatattctttccaagttttagaattggtcctaggtctggctggggcactgagcagggctgaggcctggggattgttccagtgaggaatagaccttcctcttcctaggaaaatgtcaaagacaagcttcgggccattgtagtgaccttgtcctacagtctccagacccctcggctccggcgacaggctcctggccaggggctgcctccagtggcccccatcctcaatgcccaccagcccagcacccagcgggcagaggtgagcatgggttctggtttagcccagggggaggagctgggagggcaaagatcatggtccctccccagtgacaccaattcacagctcacagagccctttcacgtatgctactccagtaattcctcatatctctaggtgcctaaatgaccatgttcctagtcaaggggacagagctcctctactccatgtagtcattcagggggctcaggttgatgggattctgtaatcttcaaatgcggccttcaagctcttgaagtcacattcatcccattcagctggaagggagaacatgaggccccttgcttgcatggtttttatggctggcccagaagtggtgcccttgacatcttttcccgttctaaatccggaactcagttacatgcatgtgagctggggcagtgtggcccagctcttgacttgctgctagtctctaccatggcatggtagtgctcccatcactaccatgatgagaggacccagaggagaggacccagttcaggatcacatggctaataagcagcagatctgagagttgaggacaggttgtttaatcctgtgatactctaacccaccatatgccatggtgacaggtttgccccctgccttgccccctaggaatgccccttatctcatgtctctccccagatccacttcctgaagcaaggctgtggtgaagacaagatctgccagagcaatctgcagctggtccgcgcccgcttctgtacccgggtcagcgacacggaattccaacctctgcccatgtgaggggggcagagagcagggtgggggtgggagggcaagagttcaaggattgagagaaagccctctcaggaggaccagtcagagggaagggctgagcctgcagaaaaggcagaaggtggaagaggacctgccgatggacttggagactgagatgagggtcagtgatggggccacgggggctcccatggcaaggaatagcaatccttcacagtacactaataatatgcagtctacatacatctcattggttagtcttcaaacctctgtgaggtaggaattatattagtctcattttatagatggagaactgaagcctagagaggttaagttacttggccaaggtaagtaatggtggagctgagatgagaacggaggtcccctgactcccagtcgtgtgtacagaggcctgaggcttggacgggtgtgatggggacctggggtgggaagagaggctcagggagtgaaggcaggtactgggggagcagactgggcgggggaatggtagggggaggtgttcagaacttagcaggagtggggtctgactctccagggatgtggatggaacaacagccctgtttgcactgagtgggcagccagtcattggcctggagctgatggtcaccaacctgccatcggacccagcccagccccaggctgatggggatgatgcccatgaagcccagctcctggtcatgcttcctgactcactgcactactcaggggtccgggccctggaccctgcggtgaggacctgggggcaggatggggtggggtcttgaggggctccagtaacccagactgaccttgccttctctcccattccaggagaagccactctgcctgtccaatgagaatgcctcccatgttgagtgtgagctggggaaccccatgaagagaggtgcccaggttggcacatctgcccttatccctacgtgagtagcctctccataagcccgtagaccacccctgactctaatctctttccacccttggcccaggtcaccttctacctcatccttagcacctccgggatcagcattgagaccacggaactggaggtagagctgctgttggccacgtaagccaggcggggccggaagggtgaggtgggggtagtcatattgacctcatctgaccccttgggaggtgcctgtgcctgatgcccatacttgcccctgcccactcaccaggatcagtgagcaggagctgcatccagtctctgcacgagcccgtgtcttcattgagctgccactgtccattgcagggtgagcctggcccaagggggcacctccattggagggagggggcattgacatttccaaaccttggccagggccctgccttcattgagccaggccccagaccttctcacccctgttctgacctctccacgccagaatggccattccccagcaactcttcttctctggtgtggtgaggggcgagagagccatgcagtctgagcgggatgtgggcagcaaggtcaagtatgaggtcacggtaagtgtcagggatgaggcccctccatggtcaccctccctccttggcacagaggagaggctgagctgtgtccccagggctggggctcttctaccatgtggcagcatgcagtttgaggcctctgtcctgcatatggaccttggctgtgggaaggtgttcctgctggggcctcatttgaccatttcctggtcattctgtctggctgtctcacctgctggtgtggtaggccatgagagtccagggaagcttctctgctgtgggcctcagctgggggatgggaacatggggcggggggttaatgtctctccctagaactctgcctttgcttggctggggcctctcctcacaccttccagggacacttccagggtttcccggatttggtgggtggagagaatggtgcctggtggggtctaggatcctgagtatttgttgaaattgtgtttcctgagggccttctcttcagcccttgttccttccatctctgtggatttaggatccacttgaaagctgagttccttggctgggcacggtggctcatgcctgtaatcacagcactacaggaggccaaggtggcaggatcacttgagcccaggagttagagacaagcctgggcaacatagatcctgtctctacaaaaaattaaaaaaaaaaaaaaaagaaaaactgaggtctctcccctcatactctctttttcccacaggtttccaaccaaggccagtcgctcagaaccctgggctctgccttcctcaacatcatgtggcctcatgagattgccaatgggaagtggttgctgtacccaatgcaggttgagctggagggcgggcaggggcctgggcagaaagggctttgctctcccaggcccaacatcctccacctggtgaggcttaggtgggggtgggggttagatgtggggacagatgttatggggagtaagggtagtgagtgcagtgatggggcaggaggaaggcggtggggaggagattcgatcttagcactgctgtgattggagggacactcactaagaccccccttccgtgtccaggatgtggacagtagggataggaggcggcgggagctggagccacctgagcagcaggagcctggtgagcggcaggagcccagcatgtcctggtggccagtgtcctctgctgagaagaagaaaaacatcaccctggtgagggcaggccagactcggtgcagccagagctccggggtgctgcgtgtccaggaagggtacctgttgagacacatgatggtctgggtgcaagttgaccagatgttcccatcacaattcataggagggggtgcccgggaggcagctactgacactgcagacccccttcctgctcagcccctgcaattgctcactctagaacaggtcactccacctgaagccagttagttagccaagcaattgattcacattacgtttatataaagcattacattttatacgtgatgggatggggttagcccatcctcacactttgaggtctgtttgtcgttcttttgaactcttataagataaaaaagtcaatttgtgtaaggacacttgcttgatttttacagtattaaaaatgttaagttcttttttgttgttgttgagacggagtcttgctctgtcaccaaggctagagtgcagtggcacgatctcagctcactgcaacctctgcctctggttcaagcgattctccttcctcagcctcctaagtagctgggactacaggtgcatgccaccacacccggctgatttttgtatttttagtagagacggggtttcaccatgttggccaggctggctggtctcgaactcctaacctcgagcgatctgtctgcctcggcctcccaaagtgctgggattataggcctgagccacgcgctcagcaaaaacactaaattctaaaagttctaataattaagacaaccgtatattatatgtgatttattaagtttaccccttttgagcagttttgctttccccttttctgcttgttttgcagagttgtgttgtttaggtgaaggtattgagcagatccgcacatgctctgtgcccctccctgctaggatggggctcgcagtgagaatgtggtggggacttagattaggaggtctttggcttgattgtaaggaggtggaggtaaaccccaggtagagccaaggagaccctttgcaggggaatgtcagcagctgctgcagtccctgctttgggccctcagctcccctgggagcatggcatgaggcaaactgaagaacgtcaggggatgtaggaacccatgtcgtggctgttgctctattacatggataatcctcccttctccacgtgaccgatttcctctgagaggcctcccttttaaatgggaactttgaaatagattctttttgttttcatgttaaaatgtatttatttatttaaatgacaaaaattatatttatcatgtataatacaatgttttgaaatatgtatatactgtggaatggctatattgagctaattaacatatgcatcacctcacatacttatttttgtggtaagaacacttaaaatctattctcttagtgattctcaggaatacaatatgttgttattaactacagtccccatgttgtgcattagatctcttaaatagatatctcttccttctgtctaactgaaactttgtatcttttgaccaccatctcctccatctccccagccacaccccgcctccactccagccctaggtaacaactattctactctctacttctatgagttcaactttcagattccacatataagtgagataatgcagtatttatctttctgaatctggcttactttgcttaacataatgtctaaagtagattcttttttattttattttatttttttattttttattttttaattttattattattatactttaagttttagggtacatgtgcacaatgtgcaggttagttacatatgtatacatgtgccatgctggtgtgctgcacccattaactcgtcatttagcattaggtatatctcctaaagctatccctccccactgcccccaccccacaacagtccccagagtgtgatgttccccttcctgtgtccatgtgttctcattgttcaattcccacctatgagtgagaatatgcggtgtttggttttttgttcttgtgatagtttagtgagaatgatttccaatttcatccatgtccctacaaaggacatgaactcatcattttttatggctgcatagtattccatggtgtatatgtgccacattttcttaatccagtctatcattgttggacatttgggttggttccaagtctttgctattgtgaatagtgccgcaataaacatacgtgtgcatgtgtctttatagcagcatgatttatagtcctttgggtatatacccagtaatgggatggctgggtcaaatggtatttctagttctagatccctgaggaatcgccacactgacttccacaaggtttgaactagtttacagtcccaccaacagtgtaaaagtgttcctatttctccacatcctctccagcacctgttgtttcctgactttttaatgattgccattctaactggtgtgagatgatatctcattgtggttttgatttgcatttctctgatggccagtgatggtgagcattttttcatgtgttttttggctgcataaatgtcttcttttgagaagtatctgttcatgtcctttgcccactttttgatggggttgtttgttttgttcttgtaaatttgtttgagttcattgtagattctggatattagccctttgtcagatgagtaggttgcgaaaattttctcccattttgtaggttgcctgttcactccgatgatagtttcttttgctgtgcagaagctctttagtttaattagatcccatttgtcaattttggcttttgttgccattgcttttggtgttttagacatgaagtccttgcccatgcctatgtcctgaatggtaattcctaggttttcttctagggtttttatggtttatgtctaacatttaagtctttaatctatcttgaattaatttttgtataaggtgttaggaagggatccagtttcagctttctacatatggctagccagttttcccagcaccatttattaaatagggaatcctttccccactggttgtttttctcaggtttgtcaaagatcagatagttgtagatatgtggcgttatttctgagggctctgttctgttccattgatctatatctctgttttggtactagtactaaagtagattcttttactaaaaaggcagaaggccttgcttttagacagcttggctaaggattcttgaatggctcaaaagtggcaaaataaaagataactcaaataattcagacactcttaaagccattcttctgtgtctggatcgtggtgtgatgaatataacataactttcaggcaaatgggtcgtgaggtggtgattgggccaaatggatttaggtcagtcctggaaagaacagagcctgagtcccctgtctacccaggagctcctgaggctacctcctcctccttcagcgagtttgggtttagggcaggggtgtggctttgccgaatatggacctgagttcaaatatttgctccaccactcattagctttgctagtgctgtgacctgaggcaggtcgcttggcctccttgggcctcctcagtttcttcctttgaaaaatgaggatgataatgtgcaccttgtaggactattgcgaagcttaagtgagataagtgcctgacaagtagtaggtgctcaataaatgggactgtcattaggtaacataagcatcacttggggaagctactctgcctgtgctgtcaagtaagagattcccttccatgctgccccctccacgcatttcaaagctgaatagctcagatttggggcaggcagctattttggatataagattagtttgagaagacttcctggaggaggggagtttgaggtcaagtaaggaaaatgggagagatggcagtctcagcagggttgagctgagtggcttagacccctgtctgtccttatccctaggactgcgcccggggcacggccaactgtgtggtgttcagctgcccactctacagctttgaccgcgcggctgtgctgcatgtctggggccgtctctggaacagcacctttctggaggtgaggataccactcggctctagtctgtgtttcttcttcctccaaagcccctccaggactcatttctcctcctccttcctcactgtccaaatgcagcatccccccccttttttttttttttgagacggagtcttcctttgtcccccaggctggagtgcagtggtgcgatctcagctcactgcaacctctgcctcccaggttcaagaggttctcctgcctcagcctcctgagtagctgagattacaggcacgtgccaccacacccgatatttttgtatttttcatagagacggggttttgccatgttggccaggctgatctcaaactcctgacctcaggtgatccaccctccttggcctcccaaagtgctgggattacaggtgtgagccactgtgcccagcccaaacgcagcatactcttaacccaaaccaggcagaaacatcctctgtgttgggcaccattacatttataatttcaggaggtaaggggaagggagggagacatggggcatttaggagaagggtctccttccctgttcccttaggagtactcagctgtgaagtccctggaagtgattgtccgggccaacatcacagtgaagtcctccataaagaacttgatgctccgagatgcctccacagtggtgagctgcagggttgggggataggtggggaggactctcttctttcctgctccctccagttcttgttccattgacctcttgctccattgaccccttgctccccagatcccagtgatggtatacttggaccccatggctgtggtggcagaaggagtgccctggtgggtcatcctcctggctgtactggctgggctgctggtgctagcactgctggtgctgctcctgtggaaggtgaggcttggaggtggggctgatgggggtggacttcctggttgggtgagcagcattttgtattgtggttctgtcacccacccacacactcatgtattcagcaaatctgcactgaacacttgccctgcatgtctcaggaactctattagtccctggatgtttcaaagataaatcagatgcaggctgggtgcagtggctcatgcctggaatctcagcactttgggaggcctaggcaggtggatcacttgaggtcaggagttcgagaccagcctggacaacatggtgaaaccccatctctacttaaaatacaaaattagctgggcgtggtggcacgcacctgtagatccagctacttgggaggctgaggcaggagaatcgattgaactcgaaaggtggggttgcagtgagccgagatggcgccattgcactccagcctgggtgacagagcgagactccatctcaaaaaaaaaaaaaaaaaaaaaagccgggcatggtggctcatgcctgtaatcccagcactttgggaggctgaggtgagtggatcacctgaggtcaggagttcaagaccaagctggccaacatggtgaaatcctgtctctgttaaaaatacaaacaaataaaaaaaattagctgggcatggtggggggtgcctgtaatcccagctacttgggaggctgagacaggagaatcgcttgaacccgggaggtggaggttgcagtgagccaagattgcgccactgcactgcaacctgggggactgagcaggactctgtctcaaaaaaaaaaaaaaaaaaaaaaaaaagaaatcagatgcagatctcgttctcagggattttatagtccagtgggatgggagtggtgagcctgtagtcaaggacaaggcaggaagtgttgagttgagaaaatggagtgggagtttagagggaaaaaagcttgagaggaggtagcactggaaccagtcctcagaggaaggtggggtagtagggtgaggatgtaaggggatgaggtaggtggggtccagcaggaccatggtaggcaaagtcgtggaggcagagaagtgtgatctgacttaactagaatgtggcagtgaaaactggaaaggtaaatgtggccaagaaggccaacaagcacggagaacggcctccaggggactgccctcatggcggaactcagagcttccacataccaggctctgcatggaagcattcttgtacacccagagctgtataccgaatcatactggtatccagggctcaatgtaaaagtatgcacatgcacacgtcagtacacggaatcacatacatcaccggggtcatatgctgagccatctgccacatcgggggctatgttcagaaccacacatgtccttggggctctggagaactgcccctgcctgtgcctgctctttgggaccaatcagagactgagccaccctttggcaagtgtgaatcatcagtagaccaggctctggagtggcatccaagctgttgggaaccttcccagtcttaggggagattgatgcctctgtgaaactggtcccaggatgcttgctccctgctcccatggaaggaagacagagcagagataggggatcctgtccttcctcaatctttccatcctctgcttcctctgccctgcttctccctcgacatcctagtgtggcttcttccatcggagcagccagagctcatcttttcccaccaactatcaccgggcctgtctggctgtgcagccttcagccatggaagttgggggtccagggactgtggggtaactgttgtgtgtgtgcatgtgtctagtgtgtgtgggtatgtatgtgtgtgaatataaggagaattcccagcattcaggggatcaggaatgttcagggtgaaaaatgagattgcatgagagtgatgtgaggtgagaggtaattagaagggtgcagatataagggtaagtctacacagcctgaggcgtgcacaaagggttggtgggctcatggggtgtgagcatgtgggagtgtggggaggtcgcatgcatgcttttttctatgggcatgagtgtgcaaggagatgaatgtgtaaggagatgccatgcagtctgagcatgtaggagtcctgggcctctgagtgtgagtgtgcaggggcacattcccaccctggtgtgtaagggccagcatggtcctcatttagggtggggctagactactgtattgatggacaagagggtattgctgtaaggggcgtgccgcccaaggtacaagtacacggggtgcatgtttatgaggcttgagaggtatcattccagagatgcaggggactaggaagccattttcttcctcaggtcttgtttgtgttctgggggacactattgggtctcttaagaagctagagggggtacaaatcttgagtccggtcctgatgctccaccctcaaggccctctttcaggggccaagggagatacccttggtcctgcctcttggacgcccttctggtctctctcttgctgtgaatgagaacctggggacacagcactgatgggagcaccgtgggtgctgcccaccacaccagatgctattcagcctatttcctttctgccgcagcagggatggtgagggtgaggagctgtgccgcgctaggcatctgctccagggaccgcagctcttcaggctcctcatggtctggcctggtgtccttatagatgggattcttcaaacgggcgaagcaccccgaggccaccgtgccccagtaccatgcggtgaagattcctcgggaagaccgacagcagttcaaggaggagaagacgggcaccatcctgaggaacaactggggcagcccccggcgggagggcccggatgcacaccccatcctggctgctgacgggcatcccgagctgggccccgatgggcatccagggccaggcaccgcctaggttcccatgtcccagcctggcctgtggctgccctccatcccttccccagagatggctccttgggatgaagagggtagagtgggctgctggtgtcgcatcaagatttggcaggatcggcttcctcaggggcacagacctctcccacccacaagaactcctcccacccaacttccccttagagtgctgtgagatgagagtgggtaaatcagggacagggccatggggtagggtgagaagggcaggggtgtcctgatgcaaaggtggggagaagggatcctaatcccttcctctcccattcaccctgtgtaacaggaccccaaggacctgcctccccggaagtgccttaacctagagggtcggggaggaggttgtgtcactgactcaggctgctccttctctagtttcccctctcatctgaccttagtttgctgccatcagtctagtggtttcgtggtttcgtctatttattaaaaaatatttgagaacaaaacctctgcctctttgagtcttgctctgg

SEQ ID NO: 86 - DNA, 4019 bp, Homo sapiens
ggagcggcgggcgggcgggagggctggcggggcgaacgtctgggagacgtctgaaagaccaacgagactttggagaccagagacgcgcctggggggacctggggcttggggcgtgcgagatttcccttgcattcgctgggagctcgcgcagggatcgtcccatggccggggctcggagccgcgacccttggggggcctccgggatttgctacctttttggctccctgctcgtcgaactgctcttctcacgggctgtcgccttcaatctggacgtgatgggtgccttgcgcaaggagggcgagccaggcagcctcttcggcttctctgtggccctgcaccggcagttgcagccccgaccccagagctggctgctggtgggtgctccccaggccctggctcttcctgggcagcaggcgaatcgcactggaggcctcttcgcttgcccgttgagcctggaggagactgactgctacagagtggacatcgaccagggagctgatatgcaaaaggaaagcaaggagaaccagtggttgggagtcagtgttcggagccaggggcctgggggcaagattgttacctgtgcacaccgatatgaggcaaggcagcgagtggaccagatcctggagacgcgggatatgattggtcgctgctttgtgctcagccaggacctggccatccgggatgagttggatggtggggaatggaagttctgtgagggacgcccccaaggccatgaacaatttgggttctgccagcagggcacagctgccgccttctcccctgatagccactacctcctctttggggccccaggaacctataattggaaggggttgctttttgtgaccaacattgatagctcagaccccgaccagctggtgtataaaactttggaccctgctgaccggctcccaggaccagccggagacttggccctcaatagctacttaggcttctctattgactcggggaaaggtctggtgcgtgcagaagagctgagctttgtggctggagccccccgcgccaaccacaagggtgctgtggttatcctgcgcaaggacagcgccagtcgcctggtgcccgaggttatgctgtctggggagcgcctgacctccggctttggctactcactggctgtggctgacctcaacagtgatggctggccagacctgatagtgggtgccccctacttctttgagcgccaagaagagctggggggtgctgtgtatgtgtacttgaaccaggggggtcactgggctgggatctcccctctccggctctgcggctcccctgactccatgttcgggatcagcctggctgtcctgggggacctcaaccaagatggctttccagatattgcagtgggtgccccctttgatggtgatgggaaagtcttcatctaccatgggagcagcctgggggttgtcgccaaaccttcacaggtgctggagggcgaggctgtgggcatcaagagcttcggctactccctgtcaggcagcttggatatggatgggaaccaataccctgacctgctggtgggctccctggctgacaccgcagtgctcttcagggccagacccatcctccatgtctcccatgaggtctctattgctccacgaagcatcgacctggagcagcccaactgtgctggcggccactcggtctgtgtggacctaagggtctgtttcagctacattgcagtccccagcagctatagccctactgtggccctggactatgtgttagatgcggacacagaccggaggctccggggccaggttccccgtgtgacgttcctgagccgtaacctggaagaacccaagcaccaggcctcgggcaccgtgtggctgaagcaccagcatgaccgagtctgtggagacgccatgttccagctccaggaaaatgtcaaagacaagcttcgggccattgtagtgaccttgtcctacagtctccagacccctcggctccggcgacaggctcctggccaggggctgcctccagtggcccccatcctcaatgcccaccagcccagcacccagcgggcagagatccacttcctgaagcaaggctgtggtgaagacaagatctgccagagcaatctgcagctggtccacgcccgcttctgtacccgggtcagcgacacggaattccaacctctgcccatggatgtggatggaacaacagccctgtttgcactgagtgggcagccagtcattggcctggagctgatggtcaccaacctgccatcggacccagcccagccccaggctgatggggatgatgcccatgaagcccagctcctggtcatgcttcctgactcactgcactactcaggggtccgggccctggaccctgcggagaagccactctgcctgtccaatgagaatgcctcccatgttgagtgtgagctggggaaccccatgaagagaggtgcccaggtcaccttctacctcatccttagcacctccgggatcagcattgagaccacggaactggaggtagagctgctgttggccacgatcagtgagcaggagctgcatccagtctctgcacgagcccgtgtcttcattgagctgccactgtccattgcaggaatggccattccccagcaactcttcttctctggtgtggtgaggggcgagagagccatgcagtctgagcgggatgtgggcagcaaggtcaagtatgaggtcacggtttccaaccaaggccagtcgctcagaaccctgggctctgccttcctcaacatcatgtggcctcatgagattgccaatgggaagtggttgctgtacccaatgcaggttgagctggagggcgggcaggggcctgggcagaaagggctttgctctcccaggcccaacatcctccacctggatgtggacagtagggataggaggcggcgggagctggagccacctgagcagcaggagcctggtgagcggcaggagcccagcatgtcctggtggccagtgtcctctgctgagaagaagaaaaacatcaccctggactgcgcccggggcacggccaactgtgtggtgttcagctgcccactctacagctttgaccgcgcggctgtgctgcatgtctggggccgtctctggaacagcacctttctggaggagtactcagctgtgaagtccctggaagtgattgtccgggccaacatcacagtgaagtcctccataaagaacttgatgctccgagatgcctccacagtgatcccagtgatggtatacttggaccccatggctgtggtggcagaaggagtgccctggtgggtcatcctcctggctgtactggctgggctgctggtgctagcactgctggtgctgctcctgtggaagatgggattcttcaaacgggcgaagcaccccgaggccaccgtgccccagtaccatgcggtgaagattcctcgggaagaccgacagcagttcaaggaggagaagacgggcaccatcctgaggaacaactggggcagcccccggcgggagggcccggatgcacaccccatcctggctgctgacgggcatcccgagctgggccccgatgggcatccagggccaggcaccgcctaggttcccatgtcccagcctggcctgtggctgccctccatcccttccccagagatggctccttgggatgaagagggtagagtgggctgctggtgtcgcatcaagatttggcaggatcggcttcctcaggggcacagacctctcccacccacaagaactcctcccacccaacttccccttagagtgctgtgagatgagagtgggtaaatcagggacagggccatggggtagggtgagaagggcaggggtgtcctgatgcaaaggtggggagaagggatcctaatcccttcctctcccattcaccctgtgtaacaggaccccaaggacctgcctccccggaagtgccttaacctagagggtcggggaggaggttgtgtcactgactcaggtttcgtggtttcgtctatttattaaaaaatatttgagaacaaaaaaaaaaaaaaaaaa

SEQ ID NO: 87 - protein, 1137 aa, Homo sapiens
MAGARSRDPWGASGICYLFGSLLVELLFSRAVAFNLDVMGALRKEGEPGSLFGFSVALHRQLQPRPQSWLLVGAPQALALPGQQANRTGGLFACPLSLEETDCYRVDIDQGADMQKESKENQWLGVSVRSQGPGGKIVTCAHRYEARQRVDQILETRDMIGRCFVLSQDLAIRDELDGGEWKFCEGRPQGHEQFGFCQQGTAAAFSPDSHYLLFGAPGTYNWKGLLFVTNIDSSDPDQLVYKTLDPADRLPGPAGDLALNSYLGFSIDSGKGLVRAEELSFVAGAPRANHKGAVVILRKDSASRLVPEVMLSGERLTSGFGYSLAVADLNSDGWPDLIVGAPYFFERQEELGGAVYVYLNQGGHWAGISPLRLCGSPDSMFGISLAVLGDLNQDGFPDIAVGAPFDGDGKVFIYHGSSLGVVAKPSQVLEGEAVGIKSFGYSLSGSLDMDGNQYPDLLVGSLADTAVLFRARPILHVSHEVSIAPRSIDLEQPNCAGGHSVCVDLRVCFSYIAVPSSYSPTVALDYVLDADTDRRLRGQVPRVTFLSRNLEEPKHQASGTVWLKHQHDRVCGDAMFQLQENVKDKLRAIVVTLSYSLQTPRLRRQAPGQGLPPVAPILNAHQPSTQRAEIHFLKQGCGEDKICQSNLQLVHARFCTRVSDTEFQPLPMDVDGTTALFALSGQPVIGLELMVTNLPSDPAQPQADGDDAHEAQLLVMLPDSLHYSGVRALDPAEKPLCLSNENASHVECELGNPMKRGAQVTFYLILSTSGISIETTELEVELLLATISEQELHPVSARARVFIELPLSIAGMAIPQQLFFSGVVRGERAMQSERDVGSKVKYEVTVSNQGQSLRTLGSAFLNIMWPHEIANGKWLLYPMQVELEGGQGPGQKGLCSPRPNILHLDVDSRDRRRRELEPPEQQEPGERQEPSMSWWPVSSAEKKKNITLDCARGTANCVVFSCPLYSFDRAAVLHVWGRLWNSTFLEEYSAVKSLEVIVRANITVKSSIKNLMLRDASTVIPVMVYLDPMAVVAEGVPWWVILLAVLAGLLVLALLVLLLWKMGFFKRAKHPEATVPQYHAVKIPREDRQQFKEEKTGTILRNNWGSPRREGPDAHPILAADGHPELGPDGHPGPGTA

SEQ ID NO: 88 - DNA, 4031 bp, Homo sapiens
ggagcggcgggcgggcgggagggctggcggggcgaacgtctgggagacgtctgaaagaccaacgagactttggagaccagagacgcgcctggggggacctggggcttggggcgtgcgagatttcccttgcattcgctgggagctcgcgcagggatcgtcccatggccggggctcggagccgcgacccttggggggcctccgggatttgctacctttttggctccctgctcgtcgaactgctcttctcacgggctgtcgccttcaatctggacgtgatgggtgccttgcgcaaggagggcgagccaggcagcctcttcggcttctctgtggccctgcaccggcagttgcagccccgaccccagagctggctgctggtgggtgctccccaggccctggctcttcctgggcagcaggcgaatcgcactggaggcctcttcgcttgcccgttgagcctggaggagactgactgctacagagtggacatcgaccagggagctgatatgcaaaaggaaagcaaggagaaccagtggttgggagtcagtgttcggagccaggggcctgggggcaagattgttacctgtgcacaccgatatgaggcaaggcagcgagtggaccagatcctggagacgcgggatatgattggtcgctgctttgtgctcagccaggacctggccatccgggatgagttggatggtggggaatggaagttctgtgagggacgcccccaaggccatgaacaatttgggttctgccagcagggcacagctgccgccttctcccctgatagccactacctcctctttggggccccaggaacctataattggaagggcacggccagggtggagctctgtgcacagggctcagcggacctggcacacctggacgacggtccctacgaggcggggggagagaaggagcaggacccccgcctcatcccggtccctgccaacagctactttggcttctctattgactcggggaaaggtctggtgcgtgcagaagagctgagctttgtggctggagccccccgcgccaaccacaagggtgctgtggttatcctgcgcaaggacagcgccagtcgcctggtgcccgaggttatgctgtctggggagcgcctgacctccggctttggctactcactggctgtggctgacctcaacagtgatggctggccagacctgatagtgggtgccccctacttctttgagcgccaagaagagctggggggtgctgtgtatgtgtacttgaaccaggggggtcactgggctgggatctcccctctccggctctgcggctcccctgactccatgttcgggatcagcctggctgtcctgggggacctcaaccaagatggctttccagatattgcagtgggtgccccctttgatggtgatgggaaagtcttcatctaccatgggagcagcctgggggttgtcgccaaaccttcacaggtgctggagggcgaggctgtgggcatcaagagcttcggctactccctgtcaggcagcttggatatggatgggaaccaataccctgacctgctggtgggctccctggctgacaccgcagtgctcttcagggccagacccatcctccatgtctcccatgaggtctctattgctccacgaagcatcgacctggagcagcccaactgtgctggcggccactcggtctgtgtggacctaagggtctgtttcagctacattgcagtccccagcagctatagccctactgtggccctggactatgtgttagatgcggacacagaccggaggctccggggccaggttccccgtgtgacgttcctgagccgtaacctggaagaacccaagcaccaggcctcgggcaccgtgtggctgaagcaccagcatgaccgagtctgtggagacgccatgttccagctccaggaaaatgtcaaagacaagcttcgggccattgtagtgaccttgtcctacagtctccagacccctcggctccggcgacaggctcctggccaggggctgcctccagtggcccccatcctcaatgcccaccagcccagcacccagcgggcagagatccacttcctgaagcaaggctgtggtgaagacaagatctgccagagcaatctgcagctggtccacgcccgcttctgtacccgggtcagcgacacggaattccaacctctgcccatggatgtggatggaacaacagccctgtttgcactgagtgggcagccagtcattggcctggagctgatggtcaccaacctgccatcggacccagcccagccccaggctgatggggatgatgcccatgaagcccagctcctggtcatgcttcctgactcactgcactactcaggggtccgggccctggaccctgcggagaagccactctgcctgtccaatgagaatgcctcccatgttgagtgtgagctggggaaccccatgaagagaggtgcccaggtcaccttctacctcatccttagcacctccgggatcagcattgagaccacggaactggaggtagagctgctgttggccacgatcagtgagcaggagctgcatccagtctctgcacgagcccgtgtcttcattgagctgccactgtccattgcaggaatggccattccccagcaactcttcttctctggtgtggtgaggggcgagagagccatgcagtctgagcgggatgtgggcagcaaggtcaagtatgaggtcacggtttccaaccaaggccagtcgctcagaaccctgggctctgccttcctcaacatcatgtggcctcatgagattgccaatgggaagtggttgctgtacccaatgcaggttgagctggagggcgggcaggggcctgggcagaaagggctttgctctcccaggcccaacatcctccacctggatgtggacagtagggataggaggcggcgggagctggagccacctgagcagcaggagcctggtgagcggcaggagcccagcatgtcctggtggccagtgtcctctgctgagaagaagaaaaacatcaccctggactgcgcccggggcacggccaactgtgtggtgttcagctgcccactctacagctttgaccgcgcggctgtgctgcatgtctggggccgtctctggaacagcacctttctggaggagtactcagctgtgaagtccctggaagtgattgtccgggccaacatcacagtgaagtcctccataaagaacttgatgctccgagatgcctccacagtgatcccagtgatggtatacttggaccccatggctgtggtggcagaaggagtgccctggtgggtcatcctcctggctgtactggctgggctgctggtgctagcactgctggtgctgctcctgtggaagatgggattcttcaaacgggcgaagcaccccgaggccaccgtgccccagtaccatgcggtgaagattcctcgggaagaccgacagcagttcaaggaggagaagacgggcaccatcctgaggaacaactggggcagcccccggcgggagggcccggatgcacaccccatcctggctgctgacgggcatcccgagctgggccccgatgggcatccagggccaggcaccgcctaggttcccatgtcccagcctggcctgtggctgccctccatcccttccccagagatggctccttgggatgaagagggtagagtgggctgctggtgtcgcatcaagatttggcaggatcggcttcctcaggggcacagacctctcccacccacaagaactcctcccacccaacttccccttagagtgctgtgagatgagagtgggtaaatcagggacagggccatggggtagggtgagaagggcaggggtgtcctgatgcaaaggtggggagaagggatcctaatcccttcctctcccattcaccctgtgtaacaggaccccaaggacctgcctccccggaagtgccttaacctagagggtcggggaggaggttgtgtcactgactcaggtttcgtggtttcgtctatttattaaaaaatatttgagaacaaaaaaaaaaaaaaaaaa

SEQ ID NO: 89 - DNA, 3426 bp, Homo sapiens
atggccggggctcggagccgcgacccttggggggcctccgggatttgctacctttttggctccctgctcgtcgaactgctcttctcacgggctgtcgccttcaatctggacgtgatgggtgccttgcgcaaggagggcgagccaggcagcctcttcggcttctctgtggccctgcaccggcagttgcagccccgaccccagagctggctgctggtgggtgctccccaggccctggctcttcctgggcagcaggcgaatcgcactggaggcctcttcgcttgcccgttgagcctggaggagactgactgctacagagtggacatcgaccagggagctgatatgcaaaaggaaagcaaggagaaccagtggttgggagtcagtgttcggagccaggggcctgggggcaagattgttacctgtgcacaccgatatgaggcaaggcagcgagtggaccagatcctggagacgcgggatatgattggtcgctgctttgtgctcagccaggacctggccatccgggatgagttggatggtggggaatggaagttctgtgagggacgcccccaaggccatgaacaatttgggttctgccagcagggcacagctgccgccttctcccctgatagccactacctcctctttggggccccaggaacctataattggaagggcacggccagggtggagctctgtgcacagggctcagcggacctggcacacctggacgacggtccctacgaggcggggggagagaaggagcaggacccccgcctcatcccggtccctgccaacagctactttggcttctctattgactcggggaaaggtctggtgcgtgcagaagagctgagctttgtggctggagccccccgcgccaaccacaagggtgctgtggttatcctgcgcaaggacagcgccagtcgcctggtgcccgaggttatgctgtctggggagcgcctgacctccggctttggctactcactggctgtggctgacctcaacagtgatggctggccagacctgatagtgggtgccccctacttctttgagcgccaagaagagctggggggtgctgtgtatgtgtacttgaaccaggggggtcactgggctgggatctcccctctccggctctgcggctcccctgactccatgttcgggatcagcctggctgtcctgggggacctcaaccaagatggctttccagatattgcagtgggtgccccctttgatggtgatgggaaagtcttcatctaccatgggagcagcctgggggttgtcgccaaaccttcacaggtgctggagggcgaggctgtgggcatcaagagcttcggctactccctgtcaggcagcttggatatggatgggaaccaataccctgacctgctggtgggctccctggctgacaccgcagtgctcttcagggccagacccatcctccatgtctcccatgaggtctctattgctccacgaagcatcgacctggagcagcccaactgtgctggcggccactcggtctgtgtggacctaagggtctgtttcagctacattgcagtccccagcagctatagccctactgtggccctggactatgtgttagatgcggacacagaccggaggctccggggccaggttccccgtgtgacgttcctgagccgtaacctggaagaacccaagcaccaggcctcgggcaccgtgtggctgaagcaccagcatgaccgagtctgtggagacgccatgttccagctccaggaaaatgtcaaagacaagcttcgggccattgtagtgaccttgtcctacagtctccagacccctcggctccggcgacaggctcctggccaggggctgcctccagtggcccccatcctcaatgcccaccagcccagcacccagcgggcagagatccacttcctgaagcaaggctgtggtgaagacaagatctgccagagcaatctgcagctggtccacgcccgcttctgtacccgggtcagcgacacggaattccaacctctgcccatggatgtggatggaacaacagccctgtttgcactgagtgggcagccagtcattggcctggagctgatggtcaccaacctgccatcggacccagcccagccccaggctgatggggatgatgcccatgaagcccagctcctggtcatgcttcctgactcactgcactactcaggggtccgggccctggaccctgcggagaagccactctgcctgtccaatgagaatgcctcccatgttgagtgtgagctggggaaccccatgaagagaggtgcccaggtcaccttctacctcatccttagcacctccgggatcagcattgagaccacggaactggaggtagagctgctgttggccacgatcagtgagcaggagctgcatccagtctctgcacgagcccgtgtcttcattgagctgccactgtccattgcaggaatggccattccccagcaactcttcttctctggtgtggtgaggggcgagagagccatgcagtctgagcgggatgtgggcagcaaggtcaagtatgaggtcacggtttccaaccaaggccagtcgctcagaaccctgggctctgccttcctcaacatcatgtggcctcatgagattgccaatgggaagtggttgctgtacccaatgcaggttgagctggagggcgggcaggggcctgggcagaaagggctttgctctcccaggcccaacatcctccacctggatgtggacagtagggataggaggcggcgggagctggagccacctgagcagcaggagcctggtgagcggcaggagcccagcatgtcctggtggccagtgtcctctgctgagaagaagaaaaacatcaccctggactgcgcccggggcacggccaactgtgtggtgttcagctgcccactctacagctttgaccgcgcggctgtgctgcatgtctggggccgtctctggaacagcacctttctggaggagtactcagctgtgaagtccctggaagtgattgtccgggccaacatcacagtgaagtcctccataaagaacttgatgctccgagatgcctccacagtgatcccagtgatggtatacttggaccccatggctgtggtggcagaaggagtgccctggtgggtcatcctcctggctgtactggctgggctgctggtgctagcactgctggtgctgctcctgtggaagatgggattcttcaaacgggcgaagcaccccgaggccaccgtgccccagtaccatgcggtgaagattcctcgggaagaccgacagcagttcaaggaggagaagacgggcaccatcctgaggaacaactggggcagcccccggcgggagggcccggatgcacaccccatcctggctgctgacgggcatcccgagctgggccccgatgggcatccagggccaggcaccgcctag

SEQ ID NO: 90 - protein, 1140 aa, Homo sapiens
MAGARSRDPWGASGICYLFGSLLVELLFSRAVAFNLDVMGALRKEGEPGSLFGFSVALHRQLQPRPQSWLLVGAPQALALPGQQANRTGGLFACPLSLEETDCYRVDIDQGADMQKESKENQWLGVSVRSQGPGGKIVTCAHRYEARQRVDQILETRDMIGRCFVLSQDLAIRDELDGGEWKFCEGRPQGHEQFGFCQQGTAAAFSPDSHYLLFGAPGTYNWKGTARVELCAQGSADLAHLDDGPYEAGGEKEQDPRLIPVPANSYFGFSIDSGKGLVRAEELSFVAGAPRANHKGAVVILRKDSASRLVPEVMLSGERLTSGFGYSLAVADLNSDGWPDLIVGAPYFFERQEELGGAVYVYLNQGGHWAGISPLRLCGSPDSMFGISLAVLGDLNQDGFPDIAVGAPFDGDGKVFIYHGSSLGVVAKPSQVLEGEAVGIKSFGYSLSGSLDMDGNQYPDLLVGSLADTAVLFRARPILHVSHEVSIAPRSIDLEQPNCAGGHSVCVDLRVCFSYIAVPSSYSPTVALDYVLDADTDRRLRGQVPRVTFLSRNLEEPKHQASGTVWLKHQHDRVCGDAMFQLQENVKDKLRAIVVTLSYSLQTPRLRRQAPGQGLPPVAPILNAHQPSTQRAEIHFLKQGCGEDKICQSNLQLVHARFCTRVSDTEFQPLPMDVDGTTALFALSGQPVIGLELMVTNLPSDPAQPQADGDDAHEAQLLVMLPDSLHYSGVRALDPAEKPLCLSNENASHVECELGNPMKRGAQVTFYLILSTSGISIETTELEVELLLATISEQELHPVSARARVFIELPLSIAGMAIPQQLFFSGVVRGERAMQSERDVGSKVKYEVTVSNQGQSLRTLGSAFLNIMWPHEIANGKWLLYPMQVELEGGQGPGQKGLCSPRPNILHLDVDSRDRRRRELEPPEQQEPGERQEPSMSWWPVSSAEKKKNITLDCARGTANCVVFSCPLYSFDRAAVLHVWGRLWNSTFLEEYSAVKSLEVIVRANITVKSSIKNLMLRDASTVIPVMVYLDPMAVVAEGVPWWVILLAVLAGLLVLALLVLLLWKMGFFKRAKHPEATVPQYHAVKIPREDRQQFKEEKTGTILRNNWGSPRREGPDAHPILAADGHPELGPDGHPGPGT